(19) United States
(12) Patent Application Publication
(10) Pub. No.: US 2013/0029846 A1
(43) Pub. Date: Jan. 31, 2013

(54) Fungicidal Iminoderivatives

(71) Applicants: Joachim Rheinheimer, Ludwigshafen (DE); Christian Pilger, Ludwigshafen (DE); Stefan Redlich, Mannheim (DE); Doris Kremzow, Heidelberg (DE); Claudia Rosenbaum, Einhausen (DE); Burghard Liebmann, Bensheim (DE); Wassilios Grammenos, Ludwigshafen (DE)

(72) Inventors: Joachim Rheinheimer, Ludwigshafen (DE); Christian Pilger, Ludwigshafen (DE); Stefan Redlich, Mannheim (DE); Doris Kremzow, Heidelberg (DE); Claudia Rosenbaum, Einhausen (DE); Burghard Liebmann, Bensheim (DE); Wassilios Grammenos, Ludwigshafen (DE)

(73) Assignee: BASF SE, Ludwigshafen (DE)

(21) Appl. No.: 13/638,090

(22) PCT Filed: Mar. 28, 2011
(86) PCT No.: PCT/EP2011/054710

§ 371 Date: Sep. 28, 2012

Related U.S. Application Data
(60) Provisional application No. 61/318,424, filed on Mar. 29, 2010.

(30) Foreign Application Priority Data
Mar. 29, 2010 (EP) 10158243.5
Mar. 7, 2011 (EP) 11157192.3

(51) Int. Cl. A01N 43/52; C07D 235/04; C07D 209/14; A01N 43/38; A01P 3/00; C07D 249/18; A01N 43/56; C07D 231/56; A61K 31/343; C07D 307/87; A01N 25/26; A01N 43/647

(52) U.S. Cl. 504/100; 514/394; 548/309.7; 548/503; 514/415; 514/359; 548/257; 514/403; 548/362.5; 514/469; 549/462

(57) ABSTRACT

The present invention relates to the use of bicyclic amidine compounds of formula I as defined in the description, and the N-oxides, and salts thereof for combating harmful fungi, and also to compositions and seed comprising at least one such compound. The invention also relates to novel bicyclic amidines and processes and intermediates for preparing these compounds.

The present invention relates to the use of bicyclic amidine compounds and the N-oxides and the salts thereof for combating phytopathogenic harmful fungi, and to compositions and seeds comprising at least one such compound. The invention also relates to novel bicyclic amidines compounds and processes for preparing these compounds.

The use of phenylamidines for controlling phytopathogenic harmful fungi is known from WO 2000/046184, WO 2003/093224, WO 2007/031508, WO 2007/031512, WO 2007/031513, WO 2007/031523, WO 2007/031524, WO 2007/031526 and WO 2009/053250.

The compounds according to the present invention differ from those described in the abovementioned publications by bearing a bicyclic ring system instead of a phenyl ring.

In many cases, in particular at low application rates, the fungicidal activity of the known fungicidal compounds is unsatisfactory. Based on this, it was an object of the present invention to provide compounds having improved activity and/or a broader activity spectrum against phytopathogenic harmful fungi.

This object is achieved by the use of certain bicyclic amidines having good fungicidal activity against phytopathogenic harmful fungi.

Accordingly, the Present invention relates to the use of compounds of formula I

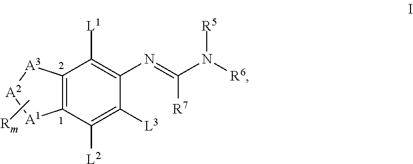

A¹, A², A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
Y is a direct bond or a divalent group selected from —O—, —S(═O)ₚ—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —(Y¹)ₖ—S(═O)ₚ—, —S(═O)ₚ—(Y¹)ₖ—, —O—Y²—, —N(R^{d})—Y²—, —O—Y²—(Y¹)ₖ—, —O—Y²—NR^{d}—NR^{d}—, —O—Y²—N(R^{d})—, —O—Y²—N(R^{d})—Y²—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—S(═O)ₚ—(Y¹)ₖ—, —(Y¹)ₖ—Y⁴—(Y¹)ₖ—O—, —(Y¹)ₖ—Y⁴—(Y¹)ₖ—S(═O)ₚ—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
   p is 0, 1 or 2,
   k is an integer between 1 and 8,
   Y¹ is —C(R^{e})₂—,
   Y² is —C(═S)— or —C(═O)—,
   Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group of N, O and S;
   R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
   R^{e} is hydrogen, CN, NH₂, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S, and wherein the aforementioned cyclic groups Rⁿ may be attached directly or via an oxygen or sulfur atom,
      or
      —[O—(ZR^{f}₂)ₙ]ₒ—R^{f}, wherein
      Z are independently of each other C or Si,
      R^{f} is hydrogen, C₁-C₈-alkyl, phenyl or benzyl,
      n is an integer between 1 and 5,
      o is an integer between 3 and 10;
R⁵,R⁶ independently of one another are C₁-C₈-alkyl, C₁-C₈-haloalkyl, C₁-C₄-alkoxy or C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S; and
   where R⁵ and R⁶ in each case together with the nitrogen atom linking them may form a five- to ten-membered saturated or partially unsaturated heterocyclyl which, in addition to the carbon atoms, may contain 1 to 3 heteroatoms from the group consisting of N, O and S;
R⁷ is hydrogen, C₁-C₈-alkyl, C₁-C₈-haloalkyl, C₁-C₈-alkoxy or C₁-C₈-haloalkoxy;
L¹,L²,L³ independently of one another are hydrogen, halogen, OH, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
   and
      aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from:
   R^{a} is amino, halogen, hydroxyl, oxo, nitro, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, NR^{A}R^{B}, C₂-C₈-alkylene, C₂-C₈-oxyalkylene, C₁-C₈-oxyalkyleneoxy, phenyl, naphthyl or a three- to ten-membered saturated, partially unsaturated or aromatic mono- or bicyclic heterocyclyl or which, in addition to carbon atoms, contains one to four heteroatoms from the group consisting of O, N and S as ring members; and wherein the aforementioned phenyl and heterocyclyl groups R^{a} are attached via a direct bond, an oxygen or sulfur atom; and
      two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group of N, O and S, and
      where the aliphatic or cyclic groups R^{a} for their part may carry 1, 2, 3 or up to the maximum possible number of identical or different groups R^{b}:
      R^{b} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
         where the cyclic groups R^{b} for their part may carry 1, 2, 3 or up to the maximum possible number of identical or different groups R^{c}:
         R^{c} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
      R^{A},R^{B} independently of one another are CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, NR^{C}R^{D}, phenyl, naphthyl or a 3- to 10-membered saturated, partially unsaturated or aromatic mono- or bicyclic heterocyclyl which, in addition to carbon atoms, contains 1 to 4 heteroatoms from the group consisting of O, N and S as ring members;
         where the cyclic groups R^{A} and/or R^{B} may for their part be attached directly or via a nitrogen or oxygen atom;
         where the aliphatic or cyclic groups R^{A} and/or R^{B} for their part may carry 1, 2, 3 or up to the maximum possible number of identical or different groups R^{b},
         R^{B} may additionally be hydrogen;
         R^{C},R^{D} independently of one another are hydrogen, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, amino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, phenyl, naphthyl or a 3- to 10-membered saturated, partially unsaturated or aromatic mono- or bicyclic heterocyclyl which, in addition to carbon atoms, contains 1 to 4 heteroatoms from the group consisting of O, N and S as ring members; where the cyclic groups R^{C} and/or R^{D} may for their part be attached directly or via a nitrogen or oxygen atom;
            and the N-oxides and the agriculturally acceptable salts of the compounds of formula I, and of compositions comprising compounds of formula I, for combating phytopathogenic fungi.

Several documents describe pharmaceutically active bicyclic amidines without a substituent in position L³ of the phenyl ring:

Certain insecticidal or acaricidal bicyclic amidines with a indazole, benzothiene benzodioxole, benzothiadiazole or benzothiazole ring are known from U.S. Pat. No. 4,186,264; U.S. Pat. No. 5,219,868; Zhurnal Organicheskoi Khimii (1987), 23(11), 2450-4; and J. Agricult. Food Chem. (1977), 25(3), 493-501.

Further bicyclic amidines with an indan or a dihydro indole ring are known as psychopharmaceutically active musarinic receptor agonists useful for the treatment of cognitive disorders, Alzheimer's disease and Schizophrenia from Eur. J. Pharmacol. (2009), 605(1-3), 53-56, Org. Proc. Res. Dev. (2009), 13(2), 198-208; Bioorg. Med. Chem. Let. (2007), 17(9), 2649-2655; WO 1997/025983; WO 1998/031660; WO 1999/004778; WO 2003/027061; WO2004/018411; WO2004/087124 and WO 2004/094363.

Further bicyclic amidines with a benzothiazole ring are known as histone deacetylase inhibitors useful for tumor treatment inter alia from WO2005/092899.

Further bicyclic amidines with an indole ring are known as pharmaceutically active tyrosine kinase inhibitors useful for tumor treatment from WO 1996/032380 or as pharmaceutically active reverse transcriptase inhibitors for treatment of HIV viral infection from Virology (1992), 190(1), 269-77.

Further bicyclic amidines with an indazole ring are known as pharmaceutically active compounds from Farmatsiya (Sofia, Bulgaria) (1977), 27(6), 1-5.

Further bicyclic amidines with a benzofuran ring are known as pharmaceutically active cell proliferation inhibitors useful for tumor treatment from Investigational New Drugs (1983), 1(2), 103-15 or as pharmaceutically active trypanosomicides useful for treatment of parasitic diseases from Liebigs Annal. Chemie (1982), (10), 1836-69

A further bicyclic amidine with a benzofuran ring has been mentioned in Antimicrob. Agents Chemotherapy (1980), 18(2), 231-9 along with test data showing that this compound has no activity at all against human fungal pathogens such as Canidida albicans.

The preparation of certain bicyclic amidines with a benzoimidazole, benzothiadiazole or benzothiene ring is generally mentioned in Khimiya Geterotsiklicheskikh Soedinenii (1986), (8), 1082-5; J. of Heterocycl. Chem. (1980), 17(7), 1441-5; and J. Chem. Res., Synopses (1978), (1), 10.

The compounds according to the present invention differ from those described in the abovementioned publications by having a mandatory substituent in position L³ attached to the phenyl ring of the bicyclic ring system with the proviso that L¹ and L³ may not be both CN, with the proviso that in case A² is N or C, one of A¹ and A³ is C, the other one of both being N, and the aforementioned atoms form together with the phenyl ring a 1H-indazole or 1H-indole, L³ may be in addition hydrogen, except for N-(1-diethoxymethyl-1H-indazol-6-yl)-N′-methyl-formamidine (CAS registry no. 65258-27-7), N-(1-diethoxymethyl-1H-indazol-5-yl)N′-methyl-formamidine (65258-39-1), N′-(1-diethoxymethyl-1H-indazol-6-yl)-N—[N-(2,4-dimethyl-phenyl)formidoyl]-N-methyl-formamidine (65259-25-8), (N′-(1-diethoxymethyl-1H-indazol-5-yl)-N—[N-(2,4-dimethyl-phenyl)-formidoyl]-N-methyl-formamidine (65258-91-5), N′-(1-diethoxymethyl-1H-indazol-6-yl)-N-[N-(4-chloro-2-methyl-phenyl)formidoyl]-N-methyl-formamidine (65531-27-3), N′-(1-diethoxymethyl-1H-indazol-5-yl)-N—[N-(4-chloro-2-methyl-phenyl)formidoyl]-N-methyl-formamidine (65258-92-6) known as insecticidal agents from U.S. Pat. No. 4,186,264, and N′-(1H-indazol-6-yl)-N,N-diethyl-formamidine (67236-60-6) known as insecticidal agent from Farmatsiya (Sofia, Bulgaria) (1977), 27(6), N′-(1,2-dimethyl-3-ethoxycarbonyl-6-hydroxy-1H-indol-5-yl)-N,N-dimethyl-formamidine (136265-46-8) known from Chem. of Heterocycl. Comp. (1991), 27(4), 369-375, and N′-(2-[[4-[(1-methylethyl)-amino]-pyrdin-2-yl]-1-piperazinyl]carbonyl)-1H-indol-5-yl)-N,N-dimethyl-formamidine (144674-89-5) known as anti-viral agent from Virology (1992), 190(1), 269-77.

Further, the preparation of 5-nnorpholinomethylideneamino-7-phenylmethylidene-indan-4,6-dicarbonitrile, a bicyclic amidine bearing CN substituents in position L¹ and L³ of the phenylring has been disclosed in Russ. J. Org. Chem. (2006), 42(3), 460-462.

The compounds according to the present invention differ from those described in Russ. J. Org. Chem. (2006), 42(3), 460-462 by the proviso that L¹ and L³ may not be both CN.

Therefore, according to a second aspect, the invention provides compounds of formula I which are represented by formula I

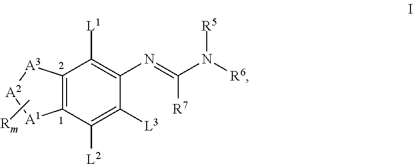

wherein:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R₁ is halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
Y is a direct bond or a divalent group selected from —O—, —S(═O)ₚ—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —(Y¹)ₖ—S(═O)ₚ—, —S(═O)ₚ—(Y¹)ₖ—, —O—Y²—, —N(R^{d})—Y²—, —O—Y²—(Y¹)ₖ—, —O—Y²—NR^{d}—NR^{d}—, —O—Y²—N(R^{d})—, —O—Y²—N(R^{d})—Y²—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—S(═O)ₚ—(Y¹)ₖ—, —(Y¹)ₖ—Y⁴—(Y¹)ₖ—O—, —(Y¹)ₖ—Y⁴—(Y¹)ₖ—S(═O)ₚ—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
   p is 0, 1 or 2,
   k is an integer between 1 and 8,
   Y¹ is —C(R^{e})₂—,
   Y² is —C(═S)— or —C(═O)—,
   Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group of N, O and S;
   R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
   R^{e} is hydrogen, CN, NH₂, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S, and wherein the aforementioned cyclic groups Rⁿ may be attached directly or via an oxygen or sulfur atom,
      or
      —[O—(ZR^{f}₂)ₙ]ₒ—R^{f}, wherein
      Z are independently of each other C or Si,
      R^{f} is hydrogen, C₁-C₈-alkyl, phenyl or benzyl,
      n is an integer between 1 and 5,
      o is an integer between 3 and 10;
R⁵,R⁶ independently of one another are C₁-C₈-alkyl, C₁-C₈-haloalkyl, C₁-C₄-alkoxy or C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S; and
   where R⁵ and R⁶ in each case together with the nitrogen atom linking them may form a five- to ten-membered saturated or partially unsaturated heterocyclyl which, in addition to the carbon atoms, may contain 1 to 3 heteroatoms from the group consisting of N, O and S;
R⁷ is hydrogen, C₁-C₈-alkyl, C₁-C₈-haloalkyl, C₁-C₈-alkoxy or C₁-C₈-haloalkoxy;
L¹,L² independently of one another are hydrogen, halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
L³ is halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
   with the proviso that L¹ and L³ may not be both CN;
   and
   with the proviso that in case A³ is N or C, one of A¹ and A³ is C, the other one of both being N, one of the bonds between A¹ and A² and between A² and A³ is a double bond and the aforementioned atoms form together with the phenyl ring a 1H-indazole or 1H-indole: L³ may in addition be hydrogen;
      and
         aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from:
   R^{a} is amino, halogen, hydroxyl, oxo, nitro, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, NR^{A}R^{B}, C₂-C₈-alkylene, C₂-C₈-oxyalkylene, C₁-C₈-oxyalkyleneoxy, phenyl, naphthyl or a three- to ten-membered saturated, partially unsaturated or aromatic mono- or bicyclic heterocyclyl or which, in addition to carbon atoms, contains one to four heteroatoms from the group consisting of O, N and S as ring members; and wherein the aforementioned phenyl and heterocyclyl groups R^{a} are attached via a direct bond, an oxygen or sulfur atom;
      and
      two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group of N, O and S, and
      where the aliphatic or cyclic groups R^{a} for their part may carry 1, 2, 3 or up to the maximum possible number of identical or different groups R^{b}:
      R^{b} is halogen, hydroxyl, nitro, CN, carboxyl, C₁-C₄-alkyl, C₂-C₄-alkenyl, C₂-C₈-alkynyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
         where the cyclic groups R^{b} for their part may carry 1, 2, 3 or up to the maximum possible number of identical or different groups R^{c}:
         R^{c} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
      R^{A},R^{B} independently of one another are CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, NR^{C}R^{D}, phenyl, naphthyl or a 3- to 10-membered saturated, partially unsaturated or aromatic mono- or bicyclic heterocyclyl which, in addition to carbon atoms, contains 1 to 4 heteroatoms from the group consisting of O, N and S as ring members;
         where the cyclic groups R^{A} and/or R^{B} may for their part be attached directly or via a nitrogen or oxygen atom;
         where the aliphatic or cyclic groups R^{A} and/or R^{B} for their part may carry 1, 2, 3 or up to the maximum possible number of identical or different groups R^{b},
         R^{B} may additionally be hydrogen;
         R^{C},R^{D} independently of one another are hydrogen, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, phenyl, naphthyl or a 3- to 10-membered saturated, partially unsaturated or aromatic mono- or bicyclic heterocyclyl which, in addition to carbon atoms, contains 1 to 4 heteroatoms from the group consisting of O, N and S as ring members; where the cyclic groups R^{C} and/or R^{D} may for their part be attached directly or via a nitrogen or oxygen atom;
            except for N-(1-diethoxymethyl-1H-indazol-6-yl)-N′-methyl-formamidine (CAS registry no. 65258-27-7), N-(1-diethoxymethyl-1H-indazol-5-yl)N′-methyl-formamidine (65258-39-1), N′-(1-diethoxymethyl-1H-indazol-6-yl)-N—[N-(2,4-dimethyl-phenyl)-formidoyl]-N-methyl-formamidine (65259-25-8), (N′-(1-diethoxymethyl-1H-indazol-5-yl)-N-[N-(2,4-dimethyl-phenyl)formidoyl]-N-methyl-formamidine (65258-91-5), N′-(1-diethoxymethyl-1H-indazol-6-yl)-N—[N-(4-chloro-2-methyl-phenyl)formidoyl]-N-methyl-formamidine (65531-27-3), N′-(1-diethoxymethyl-1H-indazol-5-yl)-N—[N-(4-chloro-2-methylphenyl)formidoyl]-N-methyl-formamidine (65258-92-6), N′-(1H-indazol-6-yl)-N,N-diethyl-formamidine (67236-60-6), N′-(1,2-dimethyl-3-ethoxycarbonyl-6-hydroxy-1H-indol-5-yl)-N,N-dimethyl-formamidine (136265-46-8) and N′-(2-[[4-[(1-methylethyl)amino]-pyrdin-2-yl]-1-piperazinyl]carbonyl)-1H-indol-5-yl)-N,N-dimethyl-formamidine (144674-89-5);
            and the N-oxides and agriculturally acceptable salts of the compounds of the formula I.

The present invention furthermore relates to processes for preparing bicyclic amidine compounds of formula I.

The present invention furthermore relates to intermediates such as compounds of formulae II, IV and V.

The present invention furthermore relates to an agrochemical composition which comprises a solid or liquid carrier and at least one compound of formula I or an N-oxide or an agriculturally acceptable salt thereof.

The compounds of the present invention are useful for combating harmful fungi. Therefore the present invention furthermore relates to a method for combating harmful fungi, which process comprises treating the fungi or the materials, plants, the soil or seeds to be protected against fungal attack, with an effective amount of at least one compound of formula I or of an N-oxide or an agriculturally acceptable salt thereof.

Furthermore, the present invention relates to seed comprising a compound of formula I, or an N-oxide or an agriculturally acceptable salt thereof, in an amount of from 0.1 g to 10 kg per 100 kg of seed.

Preferably, the invention provides compounds I, wherein
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
Y is a direct bond or a divalent group selected from —O—, —S(═O)ₚ—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —(Y¹)—S(═O)ₚ—, —S(═O)ₚ—(Y¹)ₖ—, —O—Y²—, —N(R^{d})—Y²—, —O—Y²—(Y¹)ₖ—, —O—Y²—NR^{d}—NR^{d}—, —O—Y²—N(R^{d})—, —O—Y²—N(R^{d})—Y²—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—S(═O)ₚ—(Y¹)ₖ—, —(Y¹)ₖ—Y⁴—(Y¹)ₖ—O—, —(Y¹)ₖ—Y⁴—(Y¹)ₖ—S(═O)ₚ—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
   p is 0, 1 or 2,
   k is an integer between 1 and 8,
   Y¹ is —C(R^{e})₂—,
   Y² is —C(═S)— or —C(═O)—,
   Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group of N, O and S;
   R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
   R^{e} is hydrogen, CN, NH₂, C₁-C₃-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S, and wherein the aforementioned cyclic groups Rⁿ may be attached directly or via an oxygen or sulfur atom,
      or
      —[O—(ZR^{f}₂)ₙ]ₒ—R^{f}, wherein
      Z are independently of each other C or Si,
      R^{f} is hydrogen, C₁-C₈-alkyl, phenyl or benzyl,
      n is an integer between 1 and 5,
      o is an integer between 3 and 10;
R⁵,R⁶ independently of one another are C₁-C₈-alkyl, C₁-C₈-haloalkyl, C₁-C₄-alkoxy or C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S; and
   where R⁵ and R⁶ in each case together with the nitrogen atom linking them may form a five- to ten-membered saturated or partially unsaturated heterocyclyl which, in addition to the carbon atoms, may contain 1 to 3 heteroatoms from the group consisting of N, O and S;
R⁷ is hydrogen, C₁-C₈-alkyl, C₁-C₈-haloalkyl, C₁-C₈-alkoxy or C₁-C₈-haloalkoxy;
L¹,L² independently of one another are hydrogen, halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
L³ is halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S; with the proviso that L¹ and L³ may not be both CN;
   and
      aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from:
   R^{a} is amino, halogen, hydroxyl, oxo, nitro, CN, carboxyl, C₁-C₄-alkyl, C₂-C₄-alkenyl, C₂-C₄-alkynyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, NR^{A}R^{B}, C₂-C₈-alkylene, C₂-C₈-oxyalkylene, C₁-C₈-oxyalkyleneoxy, phenyl, naphthyl or a three- to ten-membered saturated, partially unsaturated or aromatic mono- or bicyclic heterocyclyl or which, in addition to carbon atoms, contains one to four heteroatoms from the group consisting of O, N and S as ring members; and wherein the aforementioned phenyl and heterocyclyl groups R^{a} are attached via a direct bond, an oxygen or sulfur atom;
      and
      two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group of N, O and S, and
      where the aliphatic or cyclic groups R^{a} for their part may carry 1, 2, 3 or up to the maximum possible number of identical or different groups R^{b}:
      R^{b} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
         where the cyclic groups R^{b} for their part may carry 1, 2, 3 or up to the maximum possible number of identical or different groups R^{c}:
         R^{c} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
      R^{A},R^{B} independently of one another are CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, NR^{C}R^{D}, phenyl, naphthyl or a 3- to 10-membered saturated, partially unsaturated or aromatic mono- or bicyclic heterocyclyl which, in addition to carbon atoms, contains 1 to 4 heteroatoms from the group consisting of O, N and S as ring members;
         where the cyclic groups R^{A} and/or R^{B} may for their part be attached directly or via a nitrogen or oxygen atom;
         where the aliphatic or cyclic groups R^{A} and/or R^{B} for their part may carry 1, 2, 3 or up to the maximum possible number of identical or different groups R^{b},
         R^{B} may additionally be hydrogen;
         R^{C},R^{D} independently of one another are hydrogen, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂ alkyl amino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, phenyl, naphthyl or a 3- to 10-membered saturated, partially unsaturated or aromatic mono- or bicyclic heterocyclyl which, in addition to carbon atoms, contains 1 to 4 heteroatoms from the group consisting of O, N and S as ring members; where the cyclic groups R^{C} and/or R^{D} may for their part be attached directly or via a nitrogen or oxygen atom;
            and the N-oxides and agriculturally acceptable salts of the compounds of the formula I.

More preferably, the invention relates to compounds I, wherein:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
Y is a direct bond or a divalent group selected from —O—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —O—Y²—, —N(R^{d})—Y²—, —O—Y²—(Y¹)ₖ—, —O—Y²—N(R^{d})—, —O—Y²—N(R^{d})—Y²—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—(Y¹)ₖ—O—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
   p is 0, 1 or 2,
   k is an integer between 1 and 3,
   Y¹ is —C(R^{e})₂—,
   Y² is —C(═O)—,
   Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group of N, O and S;
   R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S;
   R^{e} is hydrogen, CN, NH₂, C₁-C₈-alkyl, C₂-C₈-alkenyl, C₂-C₈-alkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S, and wherein the aforementioned cyclic groups R^{e} may be attached directly or via an oxygen or sulfur atom,
      or
      —[O—(ZR^{f}₂)ₙ]ₒ—R^{f}, wherein
      Z are independently of each other C or Si,
      R^{f} is hydrogen, C₁-C₈-alkyl, phenyl or benzyl,
      n is an integer between 1 and 5,
      o is an integer between 3 and 10;
R⁵,R⁶ independently of one another are C₁-C₈-alkyl, C₁-C₈-haloalkyl, C₁-C₄-alkoxy or C₃-C₈-cycloalkyl, and where R⁵ and R⁶ in each case together with the nitrogen atom linking them may form a five- to ten-membered saturated or partially unsaturated ring;
R⁷ is hydrogen, C₁-C₈-alkyl, C₁-C₈-haloalkyl, C₁-C₈-alkoxy or C₁-C₈-haloalkoxy;
L¹,L² independently of one another are hydrogen, halogen, CN, C₁-C₆-alkyl, C₁-C₆-haloalkyl, C₁-C₆-alkoxy, C₁-C₆-haloalkoxy, di(C₁-C₈-alkyl)amino, C₁-C₆-alkylthio, C₁-C₆-haloalkylthio, C₁-C₆-alkylsulfinyl, C₁-C₆-haloalkylsulfinyl, C₁-C₈-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl;
L³ is halogen, CN, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl;
   with the proviso that Land L³ may not be both CN;
   and
      aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from:
   R^{a} is halogen, hydroxyl, oxo, CN, carboxyl, C₁-C₄-alkyl, C₂-C₄-alkenyl, C₂-C₄-alkynyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, NR^{A}R^{B}, C₂-C₈-alkylene, phenyl; and wherein the aforementioned phenyl groups R^{a} are attached via a direct bond, an oxygen or sulfur atom;
      and
      two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group of N, O and S, and
      where the aliphatic or cyclic groups R^{a} for their part may carry 1, 2, 3 or up to the maximum possible number of identical or different groups R^{b}:
      R^{b} is halogen, CN, carboxyl, C₁-C₄-alkyl, C₂-C₄-alkenyl, C₂-C₈-alkynyl, C₁-C₄-haloalkyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
         where the cyclic groups R^{b} for their part may carry 1, 2, 3 or up to the maximum possible number of identical or different groups R^{c}:
         R^{c} is halogen, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
      R^{A},R^{B} independently of one another are C₁-C₄-alkyl, C₂-C₄-alkenyl, C₂-C₄-alkynyl, C₁-C₄-haloalkyl, C₃-C₆-cycloalkyl, C₃-C₈-cycloalkenyl, phenyl; where the cyclic groups R^{A} and/or R^{B} may for their part be attached directly or via a nitrogen or oxygen atom;
         where the aliphatic or cyclic groups R^{A} and/or R^{B} for their part may carry 1, 2, 3 or up to the maximum possible number of identical or different groups R^{b},
         R^{B} may additionally be hydrogen;
         R^{C},R^{D} independently of one another are hydrogen, CN, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, phenyl,
            where the cyclic groups R^{C} and/or R^{D} may for their part be attached directly or via a oxygen atom;
               and agriculturally acceptable salts of these compounds.

According to another embodiment, the invention relates to compounds I, wherein:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is halogen, CN, oxo, C₁-C₆-haloalkyl, C₁-C₆-alkoxy, C₁-C₆-haloalkoxy, C₁-C₆-alkylthio, C₁-C₈-haloalkylthio, C₁-C₆-alkoxy-C₁-C₄-alkyl, C₁-C₆-haloalkoxy-C₁-C₄-alkyl, or Y—R⁴;
m indicates the number of the substituents R and m is 1 or 2;
R⁴ is C₁-C₈-alkyl, C₂-C₈-alkenyl, C₂-C₈-alkynyl, C₃-C₈-cycloalkyl, C₃-C₈-cycloalkyl, C₃-C₈-cycloalkenyl or phenyl;
Y is a direct bond or a divalent group selected from —O—, —(Y¹)ₖ—, —O—(Y¹)ₖ—, —O—(Y¹)ₖ—O—, —Y³—, —Y⁴—, —(Y¹)ₖ—Y³—, —O—(Y¹)ₖ—Y³—, —(Y¹)ₖ—Y⁴—, —(Y¹)ₖ—Y⁴—O—(Y¹)ₖ— and —(Y¹)ₖ—Y⁴—(Y¹)ₖ—O—, —Y⁵—; where the bond depicted on the right side of the divalent group Y is attached to R⁴, and where:
   k is an integer between 1 and 2,
   Y¹ is —C(R^{e})₂—,
   Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   R^{e} is hydrogen, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S, and wherein the aforementioned cyclic groups R^{e} may be attached directly or via an oxygen or sulfur atom,
R⁵,R⁶ independently of one another are C₁-C₈-alkyl, C₁-C₈-haloalkyl, C₁-C₄-alkoxy or C₃-C₈-cycloalkyl, and where R⁵ and R⁶ in each case together with the nitrogen atom linking them may form a five- to ten-membered saturated or partially unsaturated ring;
L¹,L² independently of one another are hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-haloalkoxy-C₁-C₄-alkyl, C₃-C₈-cycloalkyl;
L³ is halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-haloalkoxy-C₁-C₄-alkyl, C₃-C₈-cycloalkyl;
   and
      aliphatic and cyclic groups R⁴ to R⁶, Y⁵, R^{e} and L¹ to L³ may carry 1 or 2 of identical or different groups R^{a} which independently of one another are selected from:
   R^{a} is halogen, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₂-C₆-alkenyloxy, C₃-C₆-alkynyloxy, phenyl; and wherein the aforementioned phenyl groups R^{a} are attached via a direct bond, an oxygen or sulfur atom;
      and
      where the aliphatic or cyclic groups R^{a} for their part may carry 1, 2, 3 or up to the maximum possible number of identical or different groups R^{b}:
      R^{b} is halogen, CN, C₁-C₄-alkyl, C₁-C₄-haloalkyl, C₁-C₄-alkoxy, C₁-C₄-haloalkoxy, phenyl, phenoxy, pyridyl or pyridyloxy;
         where the cyclic groups R^{b} for their part may carry 1, 2, 3 or up to the maximum possible number of identical or different groups R^{c}:
         R^{c} is halogen, CN, C₁-C₄-alkyl, C₁-C₄-haloalkyl, C₁-C₄-alkoxy or C₁-C₄-haloalkoxy;
            and agriculturally acceptable salts of these compounds.

According to a further embodiment, the invention relates to compounds I, wherein:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is halogen, CN, oxo, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkoxy-C₁-C₄-alkyl, C₁-C₆-haloalkoxy-C₁-C₄-alkyl, or Y—R⁴;
m indicates the number of the substituents R and m is 1 or 2;
R⁴ is C₁-C₈-alkyl, C₂-C₈-alkenyl, C₂-C₈-alkynyl, C₃-C₈-cycloalkyl, C₃-C₈-cycloalkyl, C₃-C₈-cycloalkenyl, phenyl or a 5- to 6-membered unsaturated or aromatic monocyclic heterocyclyl wherein the ring member atoms of the heterocyclyl include besides carbon atoms 1, 2, 3 or 4 heteroatoms selected from the group of N, O and S;
Y is a direct bond or a divalent group selected from —O—, —N(R^{d})—, —(Y¹)ₖ—, —O—(Y¹)ₖ—, —O—(Y¹)ₖ—O—, —Y²—, —(Y¹)ₖ—Y²—, —Y³—, —Y⁴—, —(Y¹)ₖ—Y³—, —O—(Y¹)ₖ—Y³—, —O—Y²—N(R^{d})—, —(Y¹)ₖ—Y⁴—, —(Y¹)ₖ—Y⁴—N(R^{d})—, —(Y¹)ₖ—Y⁴—O—(Y¹)ₖ—, —(Y¹)ₖ—Y⁴—(Y¹)ₖ—O—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
   k is an integer between 1 and 3,
   Y¹ is —C(R^{e})₂—,
   Y² is —C(═O)—,
   Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁵ is C₃-C₈-cycloalkylene, phenylene,
   R^{d} is hydrogen, C₁-C₈-alkyl or phenyl;
   R^{e} is hydrogen, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S, and wherein the aforementioned cyclic groups R^{e} may be attached directly or via an oxygen or sulfur atom,
R⁵,R⁶ independently of one another are C₁-C₈-alkyl, C₁-C₈-haloalkyl, C₁-C₄-alkoxy or C₃-C₈-cycloalkyl, and where R⁵ and R⁶ in each case together with the nitrogen atom linking them may form a five- to ten-membered saturated or partially unsaturated ring;
R⁷ is hydrogen;
L¹,L² independently of one another are hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-haloalkoxy-C₁-C₄-alkyl, C₃-C₈-cycloalkyl;
L³ is halogen, CN, C₁-C₆-alkoxy, C₁-C₆-haloalkoxy, C₁-C₆-alkylthio, C₁-C₆-haloalkylthio, C₁-C₆-alkoxy-C₁-C₄-alkyl, C₁-C₆-haloalkoxy-C₁-C₄-alkyl, C₃-C₈-cycloalkyl;
   and
      aliphatic and cyclic groups R⁴ to R⁶, Y⁵, R^{d}, R^{e} and L¹ to L³ may carry 1, 2, 3, 4 or up to the maximum possible number of identical or different groups R^{a} which independently of one another are selected from:
   R^{a} is halogen, C₁-C₄-alkyl, C₂-C₄-alkenyl, C₂-C₄-alkynyl, C₁-C₄-haloalkyl, alkoxy, C₁-C₄-haloalkoxy, C₃-C₆-cycloalkyl, C₂-C₆-alkenyloxy, C₃-C₆-alkynyloxy, phenyl; and wherein the aforementioned phenyl groups R^{a} are attached via a direct bond, an oxygen or sulfur atom;
      and
      where the aliphatic or cyclic groups R^{a} for their part may carry 1, 2, 3 or up to the maximum possible number of identical or different groups R^{b}:
      R^{b} is halogen, CN, C₁-C₄-alkoxy, C₁-C₄-haloalkoxy, phenyl, phenoxy, pyridyl or pyridyloxy;
         where the cyclic groups R^{b} for their part may carry 1, 2, 3 or up to the maximum possible number of identical or different groups R^{c}:
         R^{c} is halogen, CN, C₁-C₄-haloalkyl, C₁-C₄-alkoxy or C₁-C₄-haloalkoxy;
            and agriculturally acceptable salts of these compounds.

According to a further embodiment, the invention relates to compounds I, wherein:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is halogen, CN, oxo, C₁-C₆-haloalkyl, C₁-C₆-alkoxy, C₁-C₆-haloalkoxy, C₁-C₆-alkylthio, C₁-C₆-haloalkylthio, C₁-C₆-alkoxy-C₁-C₄-alkyl, C₁-C₆-haloalkoxy-C₁-C₄-alkyl, or Y—R⁴;
m indicates the number of the substituents R and m is 1 or 2;
R⁴ is C₁-C₈-alkyl, C₂-C₈-alkenyl, C₂-C₈-alkynyl, C₃-C₈-cycloalkyl, C₃-C₈-cycloalkyl, C₃-C₈-cycloalkenyl or phenyl;
Y is a direct bond or a divalent group selected from —O—, —(Y¹)ₖ—, —O—(Y¹)ₖ—, —O—(Y¹)ₖ—O—, —Y³—, —Y⁴—, —(Y¹)ₖ—Y³—, —O—(Y¹)ₖ—Y³—, —(Y¹)ₖ—Y⁴—, —(Y¹)ₖ—Y⁴—O—(Y¹)ₖ—, —(Y¹)ₖ—Y⁴—(Y¹)ₖ—O—, —Y⁵—; where the bond depicted on the right side of the divalent group Y is attached to R⁴, and where:
   k is an integer between 1 and 2,
   Y¹ is —C(R^{e})₂—,
   Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
   R^{e} is hydrogen, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of N, O and S, and wherein the aforementioned cyclic groups R^{e} may be attached directly or via an oxygen or sulfur atom,
R⁵,R⁶ independently of one another are C₁-C₈-alkyl, C₁-C₈-haloalkyl, C₁-C₄-alkoxy or C₃-C₈-cycloalkyl, and where R⁵ and R⁶ in each case together with the nitrogen atom linking them may form a five- to ten-membered saturated or partially unsaturated ring;
R⁷ is hydrogen;
L¹,L² independently of one another are hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-haloalkoxy-C₁-C₄-alkyl, C₃-C₃-cycloalkyl;
L³ is halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkoxy-C₁-C₄-alkyl, C₁-C₆-haloalkoxy-C₁-C₄-alkyl, C₃-C₈-cycloalkyl;
   and
      aliphatic and cyclic groups R⁴ to R⁶, Y⁵, R^{e} and L¹ to L³ may carry 1 or 2 of identical or different groups R^{a} which independently of one another are selected from:
   R^{a} is halogen, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₂-C₆-alkenyloxy, C₃-C₆-alkynyloxy, phenyl; and wherein the aforementioned phenyl groups R^{a} are attached via a direct bond, an oxygen or sulfur atom;
      and
      where the aliphatic or cyclic groups R^{a} for their part may carry 1, 2, 3 or up to the maximum possible number of identical or different groups R^{b}:
      R^{b} is halogen, CN, C₁-C₄-alkyl, C₁-C₄-haloalkyl, C₁-C₄-alkoxy, C₁-C₄-haloalkoxy, phenyl, phenoxy, pyridyl or pyridyloxy;
         where the cyclic groups R^{b} for their part may carry 1, 2, 3 or up to the maximum possible number of identical or different groups R^{c}:
         R^{c} is halogen, CN, C₁-C₄-alkyl, C₁-C₄-haloalkyl, C₁-C₄-alkoxy or C₁-C₄-haloalkoxy;
            and agriculturally acceptable salts of these compounds.

The compounds I according to the invention can be obtained by different routes. Advantageously, they are prepared by reacting bicyclic amine compounds II first with an imino ester IIIa in order to obtain the intermediate cyano formamidine IIa which is then transformed into the final product I with the amine IIIb:

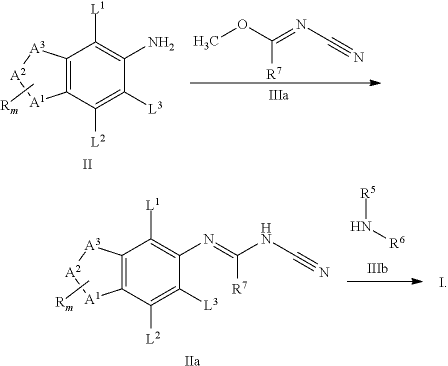

This route is described in WO 2007/031513 and can be applied here accordingly. Both consecutive reactions can be carried out at temperatures of from −20° C. to 150° C., preferably from 0° C. to 120° C., in an inert organic solvent.

Suitable solvents are aliphatic hydrocarbons such as pentane, hexane, cyclohexane and petrol ether; aromatic hydrocarbons such as toluene, o-, m- and p-xylene; halogenated hydrocarbons such as dichloromethane (DCM), chloroform and chlorobenzene; ethers such as diethyl ether, diisopropyl ether, methyl tert.-butyl ether (MTBE), dioxane, anisole and tetrahydrofuran (THF); nitriles such as acetonitrile and propionitrile; alcohols such as methanol (MeOH), ethanol (EtOH), n-propanol, isopropanol, n-butanol and tert.-butanol, and also dimethyl sulfoxide (DMSO), dimethylformamide (DMF), dimethyl acetamide, N-methyl-2-pyrrolidone (NMP), N-ethyl-2-pyrrolidone (NEP) and acetic acid ethyl ester. It is also possible to use mixtures of the solvents mentioned. The starting materials, are generally reacted with one another in equimolar amounts. In terms of yields, it may be advantageous to employ an excess of compounds IIIa and IIIb, based on the amount of compound II.

Alternatively, compounds I, wherein R⁷ is preferably is hydrogen, may be obtained by reacting compounds II preferably in the presence of catalysts, e.g. POCl₃, with compounds IIIIc:

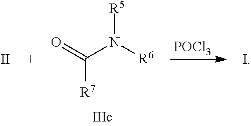

Generally, the reaction is carried out at temperatures of from −20° C. to 150° C., preferably from 20° C. to 110° C., in or without an inert organic solvent.

Suitable solvents are aromatic hydrocarbons such as chlorobenzene, nitrobenzene, toluene, o-, m- and p-xylene; halogenated hydrocarbons such as DCM, chloroform and chlorobenzene, preferably chlorinated benzenes or without solvent. It is also possible to use mixtures of the solvents mentioned.

The starting materials, are generally reacted with one another in equimolar amounts. In terms of yields, it may be advantageous to employ an excess of IIIc, based on II. An excess of phosphoryl chloride is often helpful.

Further, compounds I may also be obtained by reacting compounds II with acetal compounds IIId:

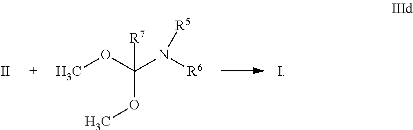

Generally, the reaction is carried out at temperatures of from −20° C. to 150° C., preferably from 0° C. to 120° C., in an inert organic solvent.

Suitable solvents are aliphatic hydrocarbons such as pentane, hexane, cyclohexane and petrol ether; aromatic hydrocarbons such as toluene, o-, m- and p-xylene; halogenated hydrocarbons such as DCM, chloroform and chlorobenzene; ethers such as diethyl ether, diisopropyl ether, MTBE, dioxane, anisole and THF; alcohols such as MeOH, EtOH, n-propanol, isopropanol, n-butanol and tert.-butanol, and also DMSO, DMF, dimethyl acetamide, NMP, NEP and acetic acid ethyl ester, it being also possible to use mixtures of these solvents. The starting materials, are generally reacted with one another in equimolar amounts. In terms of yields, it may be advantageous to employ an excess of IIId, based on II.

The compounds IIId are also known from the literature or can be obtained analogous to the known or described substances. Instead of the dimethyl acetals IIId illustrated in the scheme above, other common acetals can also be employed. These can be derived from aliphatic alcohols like EtOH or propanol or from diols like ethylene glycol or propylene glycol which yield cyclic acetals.

The amines II are known from the literature or described below or can be obtained in analogy to the known or described substances. The amines II can be prepared from the respective nitro compounds IV by reduction. Catalytic hydrogenation using for example palladium as a catalyst and reduction by reducing agents like iron or others are equally possible. The nitro compounds IV and their precursors are either known from the literature or can be prepared analogous to known compounds. The formamides IIIb are prepared from the respective amines according to standard procedures.

Compounds II are generally obtained by reduction of compounds IV:

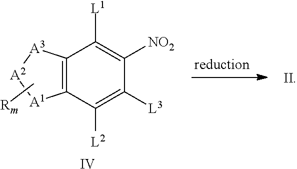

Generally, the reaction is carried out at temperatures of from −20° C. to 150° C., preferably from 0° C. to 100° C., in an inert organic solvent in presence of a reducing agent which can also be a catalyst and hydrogen.

Suitable solvents are aromatic hydrocarbons such as toluene, o-, m- and p-xylene; ethers such as diethyl ether, MTBE, dioxane, anisole and THF; alcohols such as MeOH, EtOH, n-propanol, isopropanol, n-butanol and tert.-butanol; and acetic acid ethyl ester, it being also possible to use mixtures of these solvents.

Suitable catalysts are based on palladium, platinum or nickel or on the oxides of these which may be used on a solid support. Palladium on charcoal has proved especially useful. Hydrogen can be applied from atmospheric pressure up to 150 bar, depending on the structure of IV. Preferentially pressures of up to 30 bar are used.

Compounds IV may be obtained by various alternatives.

Compounds IV, wherein A² is C, A¹, and A³ are N and form together with the phenyl ring a benzoimidazole, being referred to as compounds IVa, may be obtained by reacting benzene formamidine compounds Va in organic solvents and an acid.

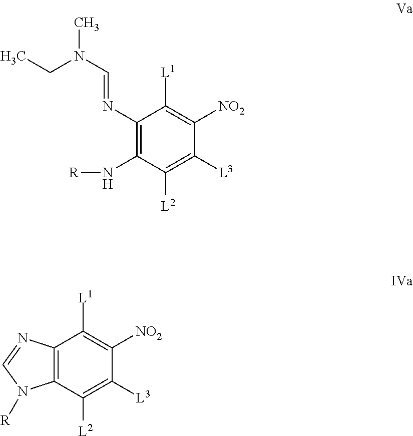

Generally, the reaction is carried out at temperatures of from −20° C. to 180° C., preferably from 20° C. to 150° C., in an inert organic solvent in presence of an acid. Suitable solvents are aliphatic hydrocarbons such as pentane, hexane, cyclohexane and petrol ether; aromatic hydrocarbons such as toluene, o-, m- and p-xylene; halogenated hydrocarbons such as DCM, chloroform and chlorobenzene; ethers such as diethyl ether, diisopropyl ether, MTBE, dioxane, anisole and THF; nitriles such as acetonitrile and propionitrile; and also DMSO, DMF, dimethyl acetamide, NMP, NEP and acetic acid ethyl ester, it also being possible to use mixtures of these solvents. Suitable acids and acidic catalysts are anorganic acids such as hydrochloric acid, hydrobromic acid, chlorosulfonic acid, sulfuric acid and perchloric acid; Lewis acids such as wie boron trifluoride, aluminium trichloride, iron(III) chloride, tin(IV) chloride, titanium(IV) chloride and zinc(II) chloride; moreover organic acids such as formic acid, acetic acid, propionic acid, oxalic acid, toluenesulfonic acid, benzenesulfonic acid, camphorsulfonic acid, citric acid and trifluoroacetic acid.

To obtain compounds Va, substituted diamino benzene compounds Vb can be reacted with acetal compounds IIId:

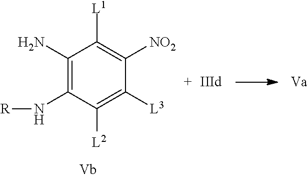

The reaction is carried out as described above for the reaction of compounds II with compounds IIb to yield compounds I.

Compounds IV, wherein A¹, A², and A³ are N and form together with the phenyl ring a benzotriazole, being referred to as compounds IVb, can be prepared in analogy to known procedures (cf. Tetrahedron 2001, 57(1), 163) starting from appropriate compounds Vb described above:

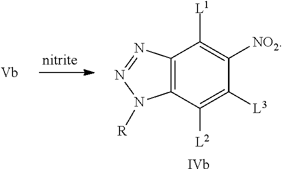

Generally, the reaction with nitrite is carried out at temperatures of from −20° C. to 120° C., preferably from −10° C. to 50° C., in water or/and an inert organic solvent in presence of an acid.

As nitrite, any inorganic (like potassium or sodium nitrite) or organic (like isoamyl nitrite) nitrites are possible, preferably sodium nitrite.

Suitable solvents are water; ethers such as dioxane and THF; alcohols such as MeOH, EtOH, n-propanol, isopropanol, preferably water. It is also possible to use mixtures of the solvents mentioned.

Suitable acids and acidic catalysts are anorganic acids such as hydrochloric acid, sulfuric acid, moreover organic acids such as formic acid, acetic acid, propionic acid, toluenesulfonic acid, benzenesulfonic acid, camphorsulfonic acid and trifluoroacetic acid.

Particular preference is given to acetic and propionic acid. The acids are generally employed in equimolar amounts, in excess or, if appropriate, as solvent.

The starting materials, are generally reacted with one another in equimolar amounts. In terms of yields, it may be advantageous to employ an excess of nitrite, based on compound Vb.

Likewise, this procedure is also suitable to obtain compounds IVb.1 starting from compounds Vb.1 applying the analogous reaction conditions:

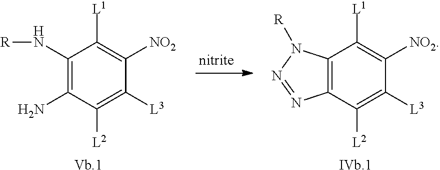

To obtain compounds Vb, substituted dinitro benzene amine compounds VIb can be hydrogenated:

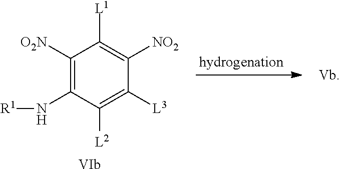

Generally, the reaction is carried out at temperatures of from 0° C. to 130° C., preferably from 0° C. to 50° C., in an inert organic solvent in presence of a catalyst. Suitable solvents are ethers such as diethyl ether, diisopropyl ether, MTBE, dioxane and THF; alcohols such as MeOH, EtOH, n-propanol, isopropanol, n-butanol and tert.-butanol, and also acetic acid ethyl ester, t being also possible to use mixtures of these solvents.

Suitable catalysts are based on palladium, platinum or nickel or on the oxides of these which may be used on a solid support. Palladium on charcoal has proved especially useful.

To obtain compounds VIb, dinitro benzene compounds VIIb in which L represents a nucleophilically replaceable leaving group such as halogen, alkylsulfonyl, alkylsulfonyloxy and arylsulfonyloxy, preferably chloro or bromo, can be reacted with amine compounds Xb, in organic solvents preferably in the presence of a base:

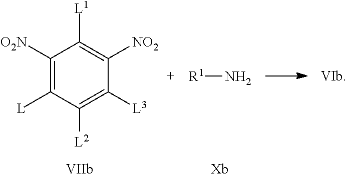

Generally, the reaction is carried out at temperatures of from 0° C. to 200° C., preferably from 20° C. to 120° C., in an inert organic solvent in presence of a base. Suitable solvents are aromatic hydrocarbons such as toluene, o-, m- and p-xylene; halogenated hydrocarbons such as chlorobenzene; ethers such as diethyl ether, diisopropyl ether, MTBE, dioxane, anisole and THF; nitriles such as acetonitrile and propionitrile; alcohols such as MeOH, EtOH, n-propanol, isopropanol, n-butanol and tert.-butanol; and also DMSO, DMF, dimethyl acetamide, NMP, NEP and acetic acid ethyl ester, it being also possible to use mixtures of these solvents.

Suitable bases are, in general, inorganic compounds, such as alkali metal and alkaline earth metal hydroxides such as lithium hydroxide, sodium hydroxide, potassium hydroxide and calcium hydroxide; alkali metal and alkaline earth metal oxides such as lithium oxide, sodium oxide, potassium oxide and calcium oxide; alkali metal and alkaline earth metal phosphates such as lithium phosphate, sodium phosphate, potassium phosphate and calcium phosphate; alkali metal amides such as lithium amide, sodium amide and potassium amide; alkali metal and alkaline earth metal hydrides lithium hydride, sodium hydride, potassium hydride and calcium hydride; alkali metal and alkaline earth metal carbonates such as lithium carbonate, potassium carbonate and calcium carbonate; alkali metal alcoholates such as sodium or potassium methylate, sodium ethylate and potassium tert.-butylate; alkali metal bicarbonates such as sodium bicarbonate and potassium bicarbonate; moreover organic bases, for example tertiary amines such as trimethylamine, triethylamine, tributylamine, diisopropylethylamine and NMP, pyridine, substituted pyridines such as collidine, lutidine and 4 dimethylaminopyridine, and also bicyclic amines. Particular preference is given to potassium carbonate, sodium hydroxide, potassium hydroxide, sodium hydride, sodium methylate and potassium tert.-butylate. The bases are generally employed in equimolar amounts, in excess or, if appropriate, as solvent.

Known phase transfer catalysts can be added in catalytic amounts in order to improve yield and reaction speed if necessary.

The starting materials, are generally reacted with one another in equimolar amounts. In terms of yields, it may be advantageous to employ an excess of Xb, based on VIIb. The educts VIIb can be obtained by the nitration of compounds which are known from the literature or can be prepared analogous to known substances.

Compounds IV, wherein A¹ is O and A² and A³ are C and these atoms from together with the phenyl ring a benzofurane, being referred to as compounds IVb, can be prepared in analogy to known procedures (cf. Eur. J. Org. Chem. 2007, 1491-1509) starting from appropriate compounds Vb as depicted below. Depending on the substituent R and on the reactions conditions used, different mixtures of isomers can be obtained:

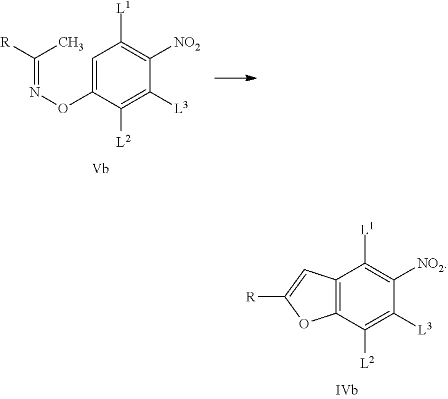

The educts Vb can be obtained from known compounds by introduction of the oxime group via nucleophilic substitution para to the nitro group analogous to the example described below.

Alternatively compounds IV, wherein A¹ is O and A² and A³ are C and these atoms from together with the phenyl ring a benzofurane, R being C(═O)—X, where X is C₁-C₈-alkoxy, being referred to as benzofurane carboxylic ester compounds IVc, can be prepared in analogy to known procedures (cf. J. Org. Chem. 1997, 8868-8874) starting from salicylic aldehydes compounds Vic:

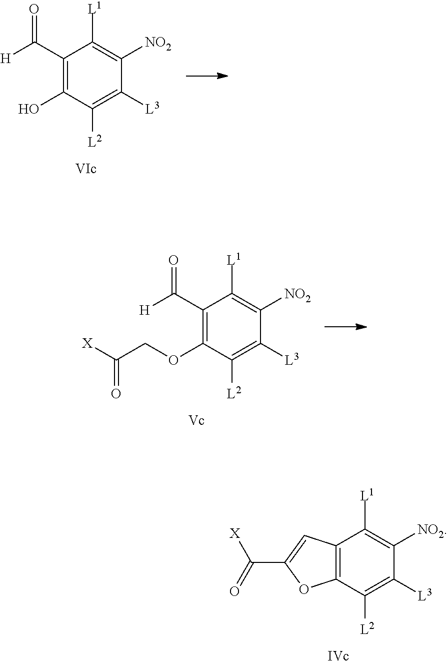

Compounds IV, V, VI, VII and X are generally known from prior art or can be obtained according to procedures known in the art.

Compounds I, wherein at least one substituent R is hydrogen, can be converted to compounds I by conventional derivatization processes such as alkylation using compounds X in which p is 1, 2 or 3 and L represents a nucleophilically replaceable leaving group such as halogen, alkylsulfonyl, alkylsulfonyloxy and arylsulfonyloxy, preferably chloro, bromo or iodo, particularly preferably chloro. Examples of suitable alkylating agents include alkyl halides, such as alkyl chloride, alkyl bromide or alkyl iodide, examples being methyl chloride, methyl bromide or methyl iodide, or dialkyl sulfates such as dimethyl sulfate or diethyl sulfate.

I: R═H+L-R→I X

Generally, the reaction is carried out at temperatures of from −20° C. to 150° C., preferably from 0° C. to 120° C., in an inert organic solvent in presence of a base. The reaction with the alkylating agent is carried out advantageously in the presence of a solvent. Suitable solvents are aliphatic hydrocarbons such as pentane, hexane, cyclohexane and petrol ether; aromatic hydrocarbons such as toluene, o-, m- and p-xylene; halogenated hydrocarbons such as DCM, chloroform and chlorobenzene; ethers such as diethyl ether, diisopropyl ether, MTBE, dioxane, anisole and THF; nitriles such as acetonitrile and propionitrile; ketones, such as acetone, methyl ethyl ketone, diethyl ketone and tert.-butyl methyl ketone; alcohols such as methanol, ethanol, n-propanol, isopropanol, n-butanol and tert.-butanol; and also DMSO, DMF, dimethyl acetamide, NMP, NEP and acetic acid ethyl ester, preferably THF, dioxane, acetonitrile, DMSO, DMF or dimethyl acetamide, it also being possible to use mixtures of these solvents.

Suitable bases are, in general, inorganic compounds, such as alkali metal and alkaline earth metal hydroxides such as lithium hydroxide, sodium hydroxide, potassium hydroxide and calcium hydroxide; alkali metal and alkaline earth metal oxides such as lithium oxide, sodium oxide, potassium oxide and calcium oxide; alkali metal and alkaline earth metal phosphates such as lithium phosphate, sodium phosphate, potassium phosphate and calcium phosphate; alkali metal amides such as lithium amide, sodium amide and potassium amide; alkali metal and alkaline earth metal hydrides lithium hydride, sodium hydride, potassium hydride and calcium hydride; alkali metal alcoholates such as sodium or potassium methylate, sodium ethylate and potassium tert.-butylate; alkali metal and alkaline earth metal carbonates such as lithium carbonate, potassium carbonate and calcium carbonate; alkali metal bicarbonates such as sodium bicarbonate and potassium bicarbonate; moreover organic bases, for example tertiary amines such as trimethylamine, triethylamine, tributylamine, diisopropylethylamine, NMP, pyridine and substituted pyridines such as collidine, lutidine and 4 dimethylaminopyridine, and also bicyclic amines.

The bases are generally employed in catalytic amounts; however, they can also be used in equimolar amounts, in excess or, if appropriate, as solvent. The amount of base is typically 1 to 1.5 molar equivalents relative to 1 mole of compound I.

The starting materials, are generally reacted with one another in equimolar amounts. In terms of yields, it may be advantageous to employ an excess of L-R, based on compound I.

The educts I are described above, whereas the compounds L-R are either known or can be prepared in analogy to known substances.

Likewise, compounds IV, wherein R is hydrogen, can be converted to compounds IV by conventional derivatization processes using compounds X as described above or in analogy to known procedures (cf. Tetrahedron 2001, 57(1), 163).

The starting materials required for preparing the compounds I are known from the literature or can be prepared in accordance with the literature cited. If individual compounds of the formula I can not be obtained by the routes described above, they can be prepared by derivatization of other compounds I.

Preferred side chains R may be obtained according to methods described in WO 09/053,250. Preferred compounds X and Xb, wherein R is defined as Y—R⁴, wherein Y is —(Y¹)ₖ—Y³—, wherein (Y¹)ₖ is —CH₂—, Y³ is —C(R^{e})═N—O— with R^{e} being methyl substituted by one group R^{a} being phenyl bound via an oxygen atom, can be prepared as follows using compounds X.2 such as epichorohydrine:

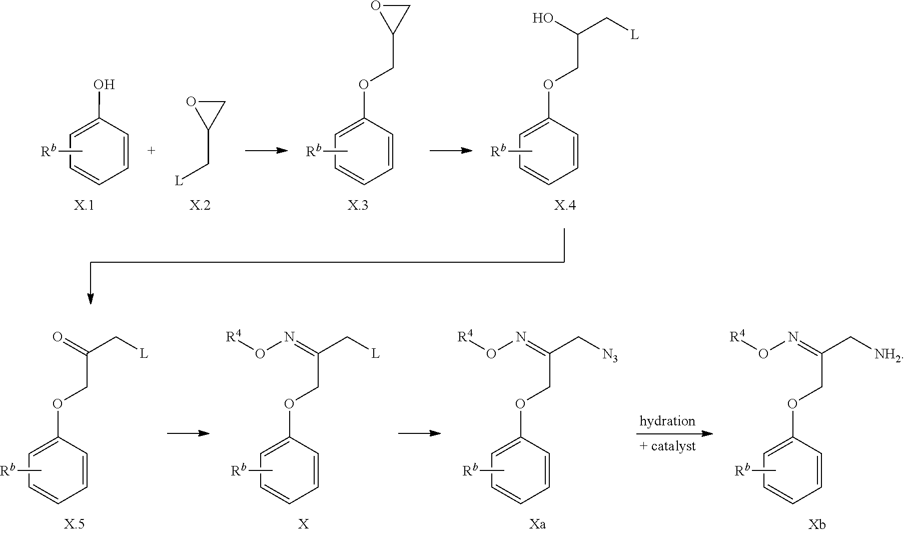

The steps of this scheme are known from literature, e.g. in Synthesis 9, 1392 (2007); J. Labelled Comp. Radiopharm. 17, 369 (1980); J. Med. Chem. 44, 1217 (2001). The oxime formation (4^{th} step in the scheme above) may performed according to standard procedures using methoxyamine hydrochloride.

The final hydrogenation step of the azide compound Xa to obtain compounds Xb may be accomplished in presence of a catalysts, such as palladium on charcoal with 1 to 50 bar hydrogen pressure in the presence of organic solvents such as alcohols.

Generally, the reaction is carried out at temperatures of from 0° C. to 150° C., preferably from 20° C. to 70° C., in an inert organic solvent in presence the hydrogenation catalysts and with the conditions mentioned above.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

The starting materials required for preparing the compounds I are known from the literature or can be prepared in accordance with the literature cited. If individual compounds of the formula I can not be obtained by the routes described above, they can be prepared by derivatization of other compounds I.

If the synthesis yields mixtures of isomers, a separation is generally not necessarily required since in some cases the individual isomers can be interconverted during preparation for application or during application (for example under the action of light, acid or bases). Such conversions may also take place after application, for example in the case of the treatment of plants in the treated plants or in the harmful fungus to be controlled.

In the definitions of the symbols given in the above formulae, collective terms were used which are generally representative for the following substituents:

halogen: fluoro, chloro, bromo and iodo;

alkyl: saturated straight-chain or branched hydrocarbon groups having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon groups having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl, such as cyclopropyl, cyclobutyl, cyclopentyl, cyclohexyl, cycloheptyl and cyclooctyl;

cycloalkenyl: mono- or bicyclic unsaturated hydrocarbon groups having 3 to 6 or 8 carbon ring members and one or two double bonds in any position, for example C₃-C₈-cycloalkenyl, such as cyclopropenyl, cyclobutenyl, cyclopentenyl, cyclohexenyl, cycloheptenyl and cyclooctenyl;

aryl or aromatic ring: a ring comprising mono-, bi- or tricyclic aromatic hydrocarbon groups and having 6, 8, 10, 12 or 14 ring members, such as phenyl, naphthyl or anthracenyl, preferably phenyl or naphthyl, in particular phenyl;

heterocyclyl: three- to ten-membered saturated, partially unsaturated or aromatic mono- or bicyclic heterocyclyl which contains one, two, three or four heteroatoms from the group consisting of O, N and S: in particular having five or six ring members:
non-aromatic saturated or partially unsaturated 5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

cyclic groups: cycloalkyl, cycloalkenyl, aryl or heterocyclyl groups as mentioned above;

alkoxy: alkyl groups as mentioned above which are attached to the skeleton via oxygen, for example C₁-C₈-alkoxy, such as OCH₃, OCH₂CH₃, O(CH₂)₂CH₃, O(CH₂)₃CH₃, O(CH₂)₄CH₃, O(CH₂)₅CH₃, O(CH₂)₆CH₃ and O(CH₂)₇CH₃;

alkenoxy: alkenyl groups as mentioned above which are attached to the skeleton via oxygen, for example C₂-C₈-alkenoxy, such as OCH═CH₂, OCH₂CH═CH₂, OCH₂CH═CHCH₃; O(CH₂)₂CH═CHCH₃ and O(CH₂)₃CH═CHCH₃;

alkynoxy: alkynyl groups as mentioned above which are attached to the skeleton via oxygen, for example C₃-C₈-alkynoxy, such as OCH₂C≡CH, O(CH₂)₂CH≡CH, O(CH₂)₃CH≡CH and O(CH₂)₄CH≡CH;

alkylcarbonyl: alkyl groups as mentioned above which are attached to the skeleton via a carbonyl group, for example C₁-C₈-alkylcarbonyl, such as COCH₃, COCH₂CH₃, CO(CH₂)₂CH₃, CO(CH₂)₃CH₃, CO(CH₂)₄CH₃, CO(CH₂)₅CH₃, CO(CH₂)₆CH₃ and CO(CH₂)₇CH₃;

alkoxycarbonyl: alkoxy groups as mentioned above which are attached to the skeleton via a carbonyl group, for example C₁-C₄-alkoxycarbonyl, such as COOCH₃, COOCH₂CH₃, COO(CH₂)₂CH₃, COO(CH₂)₃CH₃, COO(CH₂)₄CH₃, COO(CH₂)₅CH₃, COO(CH₂)₅CH₃ and COO(CH₂)₇CH₃;

alkylcarbonyloxy or alkylcarboxyl: alkylcarbonyl groups as mentioned above which are attached to the skeleton via oxo, for example C₁-C₄-alkylcarbonyloxy, such as OCOCH₃, OCOCH₂CH₃, OCO(CH₂)₂CH₃ and OCO(CH₂)₃CH₃;

alkylcarbonylamino: alkylcarbonyl groups as mentioned above which are attached to the skeleton via amino, for example C₁-C₈-alkylcarbonylamino, such as NHCOCH₃, NHCOCH₂CH₃, NHCO(CH₂)₂CH₃, NHCO(CH₂)₃CH₃, NHCO(CH₂)₄CH₃, NHCO(CH₂)₅CH₃, NHCO(CH₂)₆CH₃ and NHCO(CH₂)₇CH₃;

cycloalkylcarbonyl: cycloalkyl groups as mentioned above which are attached to the skeleton via a carbonyl group, for example C₃-C₈-cycloalkylcarbonyl, such as cyclopropylcarbonyl, cyclobutylcarbonyl, cyclopentylcarbonyl, cyclohexylcarbonyl, cycloheptylcarbonyl and cyclooctylcarbonyl;

cycloalkylcarbonylamino: cycloalkylcarbonyl groups as mentioned above which are attached to the skeleton via amino, for example C₃-C₈-cycloalkylcarbonylamino, such as cyclopropylcarbonylamino, cyclobutylcarbonylamino, cyclopentylcarbonylamino, cyclohexylcarbonylamino, cycloheptylcarbonylamino and cyclooctylcarbonylamino;

alkylene: divalent unbranched chains of 2 to 8 CH₂ groups, for example CH₂CH₂, CH₂CH₂CH₂, CH₂CH₂CH₂CH₂, CH₂CH₂CH₂CH₂CH₂, CH₂CH₂CH₂CH₂CH₂CH₂, CH₂CH₂CH₂CH₂CH₂CH₂CH₂ and CH₂CH₂CH₂CH₂CH₂CH₂CH₂CH₂;

oxyalkylene: divalent unbranched chains of 2 to 4 CH₂ groups where one valency is attached via an oxygen atom to the skeleton, for example OCH₂CH₂, OCH₂CH₂CH₂ and OCH₂CH₂CH₂CH₂;

oxyalkyleneoxy: divalent unbranched chains of 1 to 3 CH₂ groups where both valencies are attached via an oxygen atom to the skeleton, for example OCH₂O, OCH₂CH₂O and OCH₂CH₂CH₂O.

alkylidene: divalente straight-chain or branched hydrocarbon groups which have 2 to 4, 6 or 8 carbon atoms and are attached to the skeleton via a double bond, for example C₁-C₈-alkylidene, such as methylidene, ethylidene, propylidene, isopropylidene, butylidene, hexylidene and octylidene;

cycloalkylidene: cycloalkyl groups as mentioned above which are attached to the skeleton via a double bond, for example C₃-C₈-cycloalkylidene, such as cyclopropylidene, cyclobutylidene, cyclopentylidene, cyclohexylidene, cycloheptylidene and cyclooctylidene;

alkoxyimino: alkoxy groups as mentioned above which are attached to the skeleton via imino, for example C₁-C₄-alkoxyimino, such as ═NOCH₃, ═NOCH₂CH₃, ═NO(CH₂)₂CH₃, ═NO(CH₂)₃CH₃, ═NO(CH₂)₄CH₃, ═NO(CH₂)₅CH₃, ═NO(CH₂)₆CH₃ and ═NO(CH₂)₇CH₃;

and divalent groups: oxo, alkylene, oxyalkylene, oxyalkyleneoxy, alkylidene and cycloalkylidene groups as mentioned above.

Agriculturally useful salts include in particular the salts of those cations or the acid addition salts of those acids whose cations and anions, respectively, have no adverse effect on the fungicidal action of the compounds of the formula I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄)-alkylsulfonium, and sulfoxonium ions, preferably tri(C₁-C₄)-alkylsulfoxonium.

Anions of useful acid addition salts are, primarily, chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reacting the compounds of formula I with an acid of the corresponding anion, preferably hydrochloric acid, hydrobromic acid, sulfuric acid, phosphoric acid or nitric acid.

The scope of the present invention includes the (R)- and (S)-isomers and the racemates of compounds I having chiral centers.

As a result of hindered rotation of asymmetrically substituted groups, atrope isomers of compounds I may be present. They also form part of the subject matter of the invention.

The term “compounds I” refers to compounds of formula I. Likewise, this terminology applies to all sub-formulae and formulae of intermediates.

The embodiments of the intermediates and the sub-formulae of formula I with respect to the variables correspond to those of the compounds I.

With a view to the intended use of the compounds of the formula I and the compounds of all subformulae mentioned herein, such as formulae I.1A to I.15B, for example, particular preference is given to the following meanings of the substituents such as R, R¹, R², R³, R⁴, R⁵, R⁶, L¹, L², L³, Y¹, Y², Y³, R^{a}, R^{b}, R^{c}, R^{d}, R^{e}, and Z, in each case on their own or in combination:

One embodiment relates to compounds I in which at least one of A¹, A² and A³ is selected from N, O and S, more preferably A¹ is selected from N, O and S.

Another embodiment relates to compounds I in which at least one of A¹, A² and A³ is N, more preferably at least two of A¹, A² and A³ are N.

A further embodiment relates to compounds I in which A¹ is N, preferably having single bonds to neighbouring ring atoms.

A further embodiment relates compounds I in which A¹ is C.

One embodiment relates to compounds I in which A¹, A² and A³ form together with the phenyl ring a bicyclic aromatic ring, more preferably a bicyclic heteroaromatic ring.

Another embodiment relates to compounds I in which A¹, A² and A³ are C and form together with the phenyl ring an indane. These compounds correspond to formulae I.1A and I.1B, wherein R¹, R² and R³ are hydrogen or have independently of each other one of the definitions specified for R, especially those being preferred:

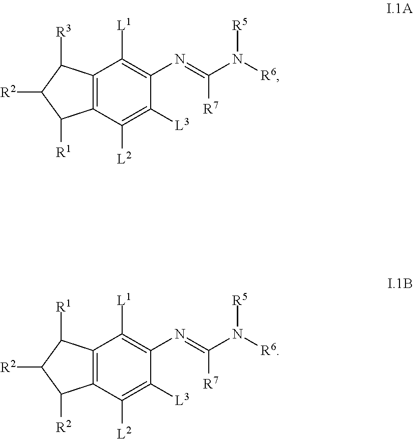

In compounds I.2, preferably R¹ is not hydrogen. In compounds I.2, both R² and both R³ are preferably hydrogen.

One embodiment relates to compounds I in which A¹, A² and A³ are C and form together with the phenyl ring a 1H-indene. These compounds correspond to the formulae I.2A and I.2B, wherein R¹, R² and R³ are hydrogen or have independently of each other one of the definitions specified for R, especially those being preferred:

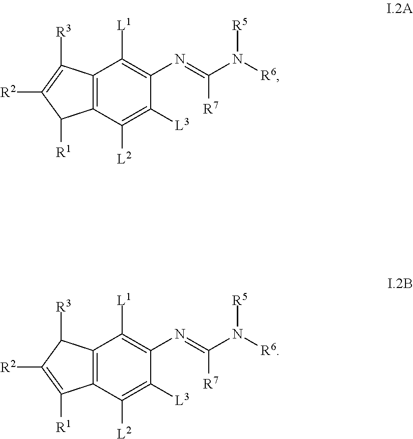

In compounds I.1A and I.1B, preferably R¹ is not hydrogen.

In compounds I.1A and I.1B, R² and R³ are preferably hydrogen.

A further embodiment relates to compounds I in which A¹ is N and A² and A³ are C, or A³ is N and A¹ and A² are C, and the aforementioned atoms form together with the phenyl ring a 2,3-dihydro-1H-indole. These compounds correspond to the formulae I.3A and I.3B, wherein R¹, R² and R³ are hydrogen or have independently of each other one of the definitions specified for R, especially those being preferred:

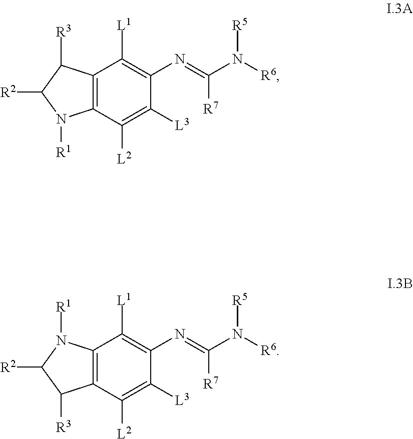

In compounds I.3A and I.3B, R² and R³ are preferably oxo, resulting in 1H-Indole-2,3-dione compounds of formulae I.3A1 and I.3B1:

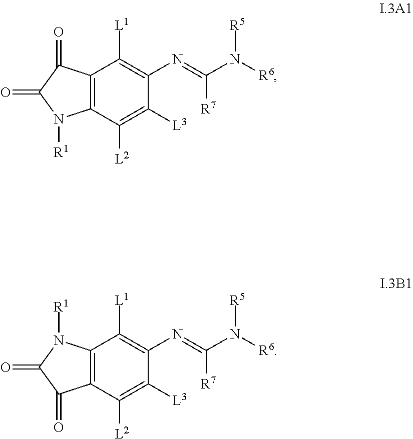

A further embodiment relates to compounds I in which A¹, A² and A³ are N and form together with the phenyl ring a 1H-benzotriazole. These compounds correspond to the formulae I.4A and I.4B wherein R¹, is hydrogen or has one of the definitions specified for R, especially those being preferred:

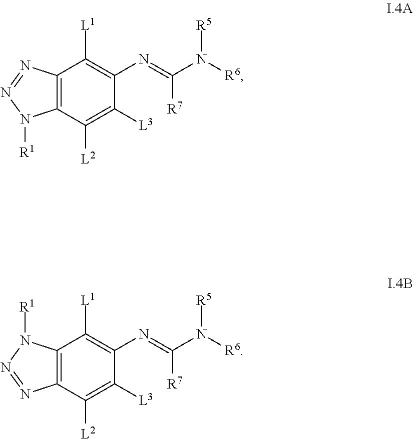

A further embodiment relates to compounds I in which A² is C, A¹ and A³ are N and the aforementioned atoms form together with the phenyl ring a 1H-benzimidazole. These compounds correspond to the formulae I.5A and I.5B, wherein R¹ and R² are hydrogen or have independently of each other one of the definitions specified for R, especially those being preferred:

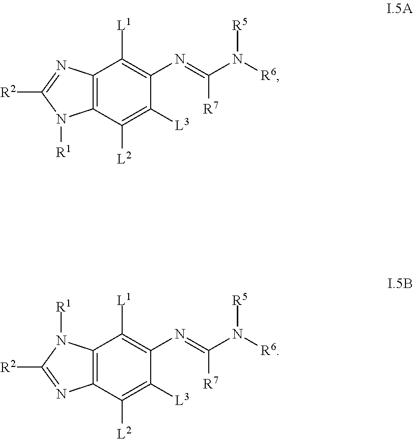

In compounds I.5A and I.5B, preferably R¹ is not hydrogen. In compounds I.5A and I.5B, R² is preferably hydrogen.

A further embodiment relates to compounds I in which A² is N, one of A¹ and A³ is C, the other one of both being N, and the aforementioned atoms form together with the phenyl ring a 1H-indazole. These compounds correspond to the formulae I.6A and I.6B, wherein R¹ and R³ are hydrogen or have independently of each other one of the definitions specified for R, especially those being preferred:

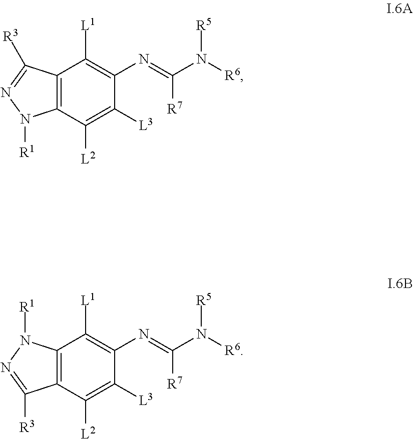

In compounds I.6A and I.6B, preferably R¹ is not hydrogen. In compounds I.6A and I.6B, R³ is preferably hydrogen.

A further embodiment relates to compounds I in which A¹ is O, A² and A³ are C, or A³ is O, A¹ and A² are C, and the aforementioned atoms form together with the phenyl ring a benzofurane. These compounds correspond to the formulae I.7A and I.7B, wherein R¹ and R³ are hydrogen or have independently of each other one of the definitions specified for R, especially those being preferred:

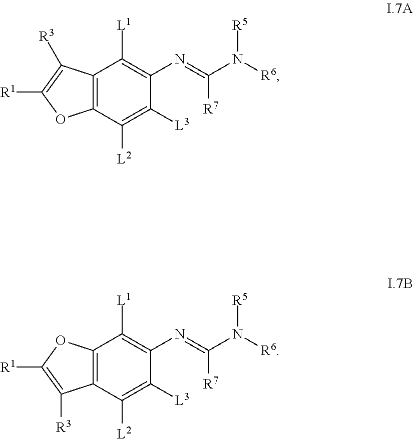

A further embodiment relates to compounds I in which A¹ is S, A² and A³ are C, or A³ is S, A¹ and A² are C, and the aforementioned atoms form together with the phenyl ring a benzothiophene. These compounds correspond to the formulae I.8A and I.8B, wherein R¹ and R³ have independently of each other one of the definitions specified for R, especially those being preferred:

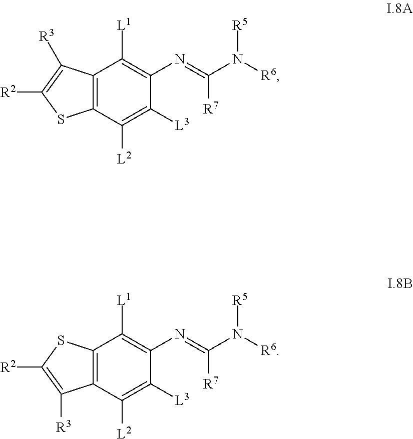

A further embodiment relates to compounds I in which A² is O, A¹ and A³ are C and the aforementioned atoms form together with the phenyl ring an isobenzofurane. These compounds correspond to the formulae I.9A and I.9B, wherein R¹ and R³ are hydrogen or have independently of each other one of the definitions specified for R, especially those being preferred:

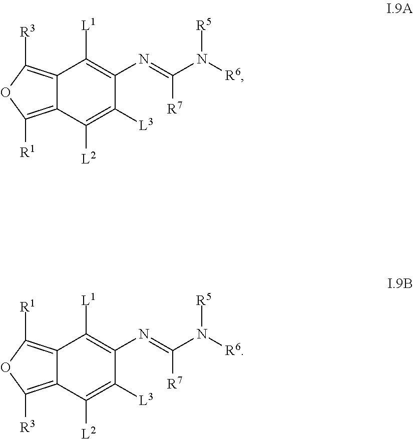

A further embodiment relates to compounds I in which A² is C, one of A¹ and A³ is N, the other one of both being O, And the aforementioned atoms form together with the phenyl ring a benzoxazole, preferably A¹ is N and A³ is O. These compounds correspond to the formulae I.10A and I.10B, wherein R² is hydrogen or has one of the definitions specified for R, especially those being preferred:

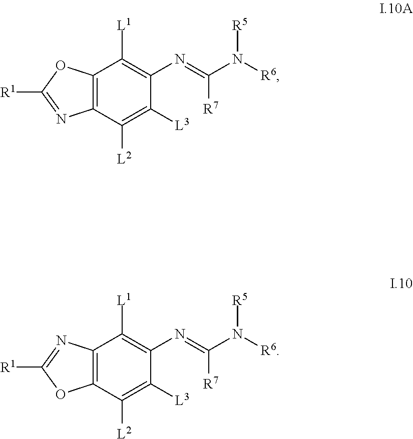

A further embodiment relates to compounds I in which A² is N, one of A¹ and A³ is C, the other one of both being O, and the aforementioned atoms form together with the phenyl ring a benzisoxazole. These compounds correspond to the formulae I.11A and I.11B, wherein R¹ is hydrogen or has one of the definitions specified for R, especially those being preferred:

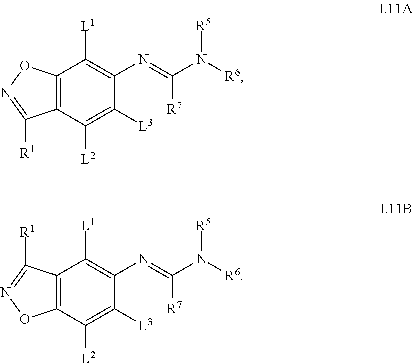

A further embodiment relates to compounds I in which A² is O, one of A¹ and A³ is C, the other one of both being N, and the aforementioned atoms form together with the phenyl ring a 2,1-benzisoxazole. These compounds correspond to the formula I.12A and I.12B, wherein R¹ is hydrogen or has one of the definitions specified for R, especially those being preferred:

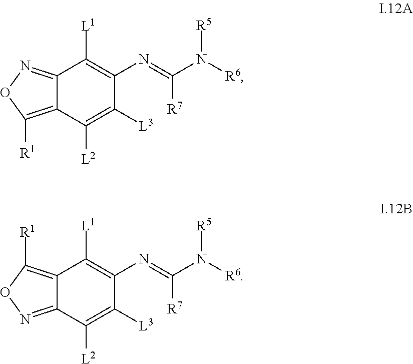

A further embodiment relates to compounds I in which A² is C, one of A¹ and A³ is C, the other one of both being N, and the aforementioned atoms form together with the phenyl ring a 1H-indole. These compounds correspond to the formulae I.13A and I.13B, wherein R¹, R² and R³ are hydrogen or have independently of each other one of the definitions specified for R, especially those being preferred:

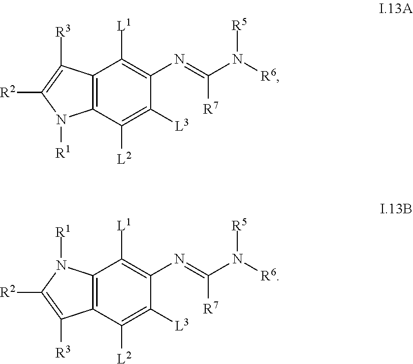

A further embodiment relates to compounds I in which A² is C, one of A¹ and A³ is C, the other one of both being O, and the aforementioned atoms form together with the phenyl ring a 2,3-dihydro-benzofurane (coumaran). These compounds correspond to the formulae I.14A and I.14B, wherein R¹ and R³ are hydrogen or have independently of each other one of the definitions specified for R, especially those being preferred:

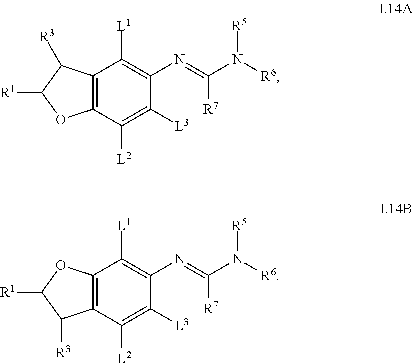

A further embodiment relates to compounds I in which A² is N, one of A¹ and A³ is C, the other one of both being N, and the aforementioned atoms form together with the phenyl ring a 2H-indazole. These compounds correspond to the formulae I.15A and I.15B, wherein R¹ and R³ are hydrogen or have independently of each other one of the definitions specified for R, especially those being preferred:

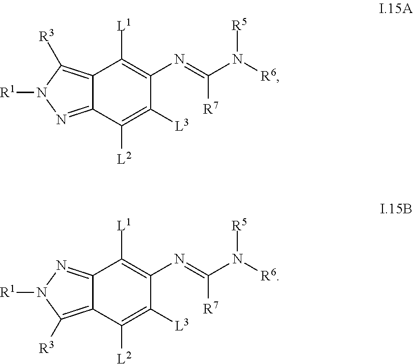

One embodiment relates to compounds I in which R bound to A¹ is not hydrogen. Another embodiment relates to compounds I.1A to I.14B, wherein R¹ is not hydrogen.

A further embodiment relates compounds I selected from the sub-formulae I.1A, I.1B, I.2A, I.2B, I.3A, I.3B, I.4A, I.4B, I.5A, I.5B, I.6A, I.6B, I.7A, I.7B, I.11A, I.11B, I.13A, I.13B, I.14A, I.14B, I.15A and I.15B, wherein A¹, A² and A³ are as defined in the subformulae and R¹, R² and R³ are hydrogen or have independently of each other one of the definitions specified for R as defined herein.

In the compounds I.1A to I.15B, R² and R³, where applicable, are preferably independently of each other hydrogen, halogen, C₁-C₆-alkyl or C₁-C₆-haloalkyl, more prefably hydrogen, methyl or CF₃.

In the compounds according to the invention, R is preferably Y—R⁴.

In the compounds according to the invention, Y is preferably a direct bond or a divalent group selected from —O—, —(Y¹)ₖ—, —O—(Y¹)ₖ—, and —O—(Y¹)ₖ—O—.

Another embodiment relates to compounds I in which Y is preferably a divalent group selected from —(Y₁)ₖ—Y³—, —O—(Y¹)ₖ—Y³—, and —S—(Y¹)ₖ—Y³—, in particular —(Y¹)ₖ—Y³—.

Another embodiment relates to compounds I in which R² is —[O—(ZH₂)ₙ]ₒ—H, wherein
Z are independently of each other carbon or silicium;
n is an integer between 1 and 5,
o is an integer between 3 and 10.

Another embodiment relates to compounds I, wherein R^{e} is phenoxy, heterocyclyloxy, phenylthio, heterocyclylthio or C₁-C₁₂-alkylthio, more preferably R^{e} is phenoxy.

A further embodiment relates to compounds, wherein R^{e} is C₁-C₈-alkylthio. Further embodiments relate to compounds I in which R^{e} is in each case one of the following groups E-1 to E-21 in table E.

TABLE E

| No. | R^{e} |
|---|---|
| E-1 | 3,4-dichloroanilinyl |
| E-2 | 2-trifluoromethylphenylhydrazinyl |
| E-3 | 2-chloro-4-trifluoromethylphenylhydrazinyl |
| E-4 | 2,4-dichlorophenylhydrazinyl |
| E-5 | 1N-methyl-1N-penylhydrazinyl |
| E-6 | thioethyl |
| E-7 | n-propylamino |
| E-8 | 3-chloro-5-trifluoromethyl-2-pyridylamino |
| E-9 | (2-methoxyethyl)-amino |
| E-10 | 4-chlorobenzylamino |
| E-11 | N,N-bis-(4-chlorobenzyl)-amino |
| E-12 | N,N-dimethylamino |
| E-13 | methylamino |
| E-14 | (cyclopropylmethyl)-amino |
| E-15 | N,N-bis-(2,4-dichlorobenzyl)-amino |
| E-16 | 2,4-dichlorobenzylamino |
| E-17 | 2,4-dichlorophenylamino |
| E-18 | 2,4,6-trimethylphenylamino |
| E-19 | 2,6-dichlorophenylthio |
| E-20 | 2-chloro-4-fluorophenylthio |
| E-21 | 1N-ethyl-1N-phenylhydrazinyl |

A further embodiment relates to compounds I, wherein R is defined as

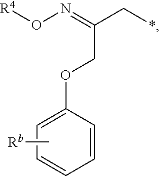

wherein * indicates the bond to one of the atoms A¹, A² or A³. Preferably, the aforementioned specific R group is bound to A¹, more preferably A¹ is then N.

In the compounds according to the invention, R⁴ is preferably C₁-C₈-alkyl, C₂-C₈-alkenyl, C₂-C₈-alkynyl, C₃-C₈-cycloalkyl, phenyl or a five- or six-membered saturated, partially unsaturated or aromatic heterocyclyl which, in addition to carbon atoms, contains one to three heteroatoms from the group consisting of O, N and S as ring members, where R⁴ may carry 1, 2, 3 or up to the maximum possible number of identical or different groups R^{a} which are not heterocyclic groups.

One embodiment relates to compounds I in which R⁴ is optionally R^{a}-substituted C₁-C₈-alkyl, C₂-C₈-alkenyl, C₃-C₈-cycloalkyl, phenyl, pyridyl, pyrazolyl, imidazolyl or triazolyl. A further embodiment relates to compounds I in which R⁴ is optionally R^{a}-substituted C₁-C₈-alkyl, C₂-C₈-alkenyl, C₃-C₈-cycloalkyl or phenyl. A further embodiment relates to compounds I in which R⁴ is optionally R^{a}-substituted C₁-C₈-alkyl. A further embodiment relates to compounds I in which R⁴ is optionally R^{a}-substituted C₂-C₈-alkenyl. A further embodiment relates to compounds I in which R⁴ is optionally R^{a}-substituted C₃-C₈-cycloalkyl. A further embodiment relates to compounds I in which R⁴ is optionally R^{a}-substituted phenyl. A further embodiment relates to compounds I in which the α carbon atom in R⁴ does not carry a cyclic group. A further embodiment relates to compounds I in which R⁴ is optionally R^{a}-substituted C₁-C₄-alkyl or C₂-C₄-alkenyl. A further embodiment relates to compounds I in which R⁴ is optionally R^{a}-substituted methyl.

A further embodiment relates to compounds I in which R⁴ is unsubstituted.

Further embodiments relate to compounds I in which R⁴ is in each case one of the following groups IV-1 to I-19 in table IV:

TABLE IV

| No. | R⁴ |
|---|---|
| IV-1 | CH₃ |
| IV-2 | CH₂CH₃ |
| IV-3 | CH(CH₃)₂ |
| IV-4 | CH₂CH₂CH₃ |
| IV-5 | CH₂CF₃ |
| IV-6 | CHF₂ |
| IV-7 | CH₂OCH₃ |
| IV-8 | CH₂CH₂CH₂CH₃ |
| IV-9 | CH(CH₃)CH₂CH₃ |
| IV-10 | CH₂CH(CH₃)₂ |
| IV-11 | (S)—CHCF₃CH₃ |
| IV-12 | (R)—CHCF₃CH₃ |
| IV-13 | cyclopropylmethyl |
| IV-14 | 2-buten-1-yl |
| IV-15 | 3-methyl-2-buten-1-yl |
| IV-16 | propargyl |
| IV-17 | 1-butyn-3-yl |
| IV-18 | 2-butyn-1-yl |
| IV-19 | E-3-chloro-2-propen-1-yl |

Preferably, R⁵ and R⁶ in the compounds I according to the invention are C₁-C₄-alkyl which may carry one, two, three, four or five identical or different groups R^{a}.

One embodiment relates to compounds I in which R⁵ is methyl or ethyl, in particular R⁵ is methyl.

One embodiment relates to compounds I in which R⁶ is ethyl.

One embodiment relates to compounds I in which R⁷ is hydrogen or C₁-C₈-alkyl, more preferably hydrogen, methyl or ethyl, in particular hydrogen.

Further embodiments relate to compounds I in which R⁵, R⁶ and R⁷ are one of the following combinations R-1 to R-43 in table R:

TABLE R

| No. | R⁷ | R⁵ | R⁶ |
|---|---|---|---|
| R-1 | H | CH₃ | CH₂CH₃ |
| R-2 | H | CH₃ | CH₂CF₃ |
| R-3 | H | CH₃ | CH₃ |
| R-4 | H | CH₃ | CH(CH₃)₂ |
| R-5 | H | CH₃ | CHF₂ |
| R-6 | H | CH₃ | CF₃ |
| R-7 | H | CH₃ | CH₂CH₂CH₃ |
| R-8 | H | CH₃ | CH₂OCH₃ |
| R-9 | H | CH₃ | C(CH₃)₃ |
| R-10 | H | CH₃ | OCH₃ |
| R-11 | H | CH₃ | OCH₂CH₃ |
| R-12 | H | CH₃ | OCH₂CH₂CH₃ |
| R-13 | H | CH₃ | OCH(CH₃)₂ |
| R-14 | H | CH₃ | OCHF₂ |
| R-15 | H | CH₃ | CH₂CH₂CH₃ |
| R-16 | H | CH₃ | CH₂CH₂CF₃ |
| R-17 | H | CH₃ | CH₂CHF₂ |
| R-18 | H | CH₃ | CH₂CH₂CH₂CH₃ |
| R-19 | H | CH₃ | CH(CH₃)CH₂CH₃ |
| R-20 | H | CH₃ | CH₂CH(CH₃)₂ |
| R-21 | H | CH₃ | (S)-CHCF₃CH₃ |
| R-22 | H | CH₃ | (R)-CHCF₃CH₃ |
| R-23 | H | CH₃ | cyclopropylmethyl |
| R-24 | H | CH₃ | 2-buten-1-yl |
| R-25 | H | CH₃ | 3-methyl-2-buten-1-yl |
| R-26 | H | CH₃ | propargyl |
| R-27 | H | CH₃ | 1-butyn-3-yl |
| R-28 | H | CH₃ | 2-butyn-1-yl |
| R-29 | H | CH₃ | E-3-chloro-2-propen-1-yl |
| R-30 | H | CH₃ | cyclopropyl |
| R-31 | H | CH₃ | cyclopropylmethyl |

| | | |
|---|---|---|
| R-32 | H | —CH₂CH₂CH₂CH₂— |
| R-33 | H | —(S)—CH(CH₃)CH₂CH₂CH₂— |
| R-34 | H | —(R)—CH(CH₃)CH₂CH₂CH₂— |
| R-35 | H | —CH(CH₃)CH₂CH₂CH(CH₃)— |
| R-36 | H | —CH₂CH₂CH₂CH₂CH₂— |
| R-37 | H | —(S)—CH(CH₃)CH₂CH₂CH₂CH₂— |
| R-38 | H | —(R)—CH(CH₃)CH₂CH₂CH₂CH₂— |
| R-39 | H | —CH(CH₃)CH₂CH₂CH₂CH(CH₃)— |
| R-40 | H | —CH₂CH₂CH(CH₃)CH₂CH₂— |
| R-41 | H | —CH₂CH₂OCH₂CH₂— |
| R-42 | H | —CH₂CH₂N(CH₃)CH₂CH₂— |
| R-43 | H | —CH₂N(CH₃)CH₂CH₂CH₂— |

One embodiment relates to compounds I in which L³ is not hydrogen.

Another preferred embodiment relates to compounds I in which L¹ and L³ may not be both CN.

One embodiment relates to compounds I in which L¹ and L² independently of one another are halogen, C₁-C₄-alkyl, C₁-C₄-haloalkyl or C₁-C₄-haloalkoxy, even more preferably selected from the group of CH₃, CF₃, OCHF₂ and OCF₃.

Another embodiment relates to compounds I in which L¹ and L² independently of one another are halogen, such as chloro or fluoro.

A further embodiment relates to compounds I in which L¹ and L² independently of one another are methyl or halomethyl, such as CF₃, CCl₃, CH₂Cl, CH₂F, CHF₂ or CHCl₂.

A further embodiment relates to compounds in which L¹ and L² are methyl.

A further embodiment relates to compounds I in which L¹ and L² independently of one another are C₁-C₄-haloalkoxy, preferably OCHF₂ or OCF₃.

A further embodiment relates to compounds in which L¹ and L² are hydrogen.

Further embodiments relate to compounds I in which L¹, L² and L³ are one of the following combinations L-1 to L-4 in table L:

TABLE L

| No. | L¹ | L² | L³ |
|---|---|---|---|
| L-1 | H | H | CH₃ |
| L-2 | H | H | CH₂CH₃ |
| L-3 | H | H | Cl |
| L-4 | H | H | F |

In the compounds according to the invention, R^{a} is preferably halogen, C₁-C₄-alkyl, C₁-C₄-haloalkyl, C₁-C₄-alkoxy, C₁-C₄-haloalkoxy, phenyl or a five- or six-membered saturated, partially unsaturated or aromatic heterocyclyl which, in addition to carbon atoms, contains one to three heteroatoms from the group consisting of O, N and S as ring members, where the cyclic groups R^{a} may be attached directly or via a nitrogen or oxygen atom; where the aliphatic and cyclic groups R^{a} for their part may carry one, two, three or up to the maximum possible number of identical or different groups R^{b}, where R^{b} is halogen, C₁-C₄-alkyl, C₁-C₄-haloalkyl, C₁-C₄-alkoxy or C₁-C₄-haloalkoxy.

In the compounds according to the invention, R^{a} is in particular halogen, C₁-C₄-alkyl, C₁-C₄-alkoxy, C₁-C₄-haloalkoxy, phenyl, pyridyl, pyrimidinyl, pyridazinyl, triazinyl, pyrazolyl, imidazolyl or triazolyl, where the cyclic groups R^{a} may be attached directly or via an oxygen atom, where the aromatic and heterocyclic groups R^{a} for their part may carry one, two, three or up to the maximum possible number of identical or different groups R^{b}, where R^{b} is halogen, C₁-C₄-alkyl, C₁-C₄-haloalkyl, C₁-C₄-alkoxy or C₁-C₄-haloalkoxy.

In a further preferred embodiment, R^{a} in the compounds according to the invention is halogen, C₁-C₄-alkyl, C₁-C₄-alkoxy, C₁-C₄-haloalkoxy, phenyl or pyridyl, where the cyclic groups R^{a} may be attached directly or via an oxygen atom, where the aromatic and heterocyclic groups R^{a} for their part may carry one, two, three or up to the maximum possible number of identical or different groups R^{b}, where R^{b} is halogen, C₁-C₄-alkyl, C₁-C₄-haloalkyl, C₁-C₄-alkoxy or C₁-C₄-haloalkoxy.

A skilled person will readily understand that the preferences given in connection with compounds I apply for formulae I.1A to I.15B as defined above.

With respect to their use, particular preference is given to the compounds of formulae I.1A to I.15B, wherein the definitions for the substituents L¹, L² and L³ of the phenyl group are selected from L-1 to L-4 in Table L and wherein the definitions for groups R⁵, R⁶ and R⁷ are selected from R-1 to R-43 in Table R and wherein the definitions for R¹, R² and R³ are selected from table A. Here, the groups mentioned in the Tables for a substituent are furthermore, independently of the combination wherein they are mentioned, a particularly preferred embodiment of the substituent in question.

TABLE A

| No. | R¹ | R² | R³ |
|---|---|---|---|
| 1 | 2,6-difluorobenzyl | H | H |
| 2 | 2-chloro-6-fluorobenzyl | H | H |
| 3 | 2,4-dichlorobenzyl | H | H |
| 4 | 2,4,6-trifluorobenzyl | H | H |
| 5 | 2,6-dichlorobenzyl | H | H |
| 6 | 3,4-difluorobenzyl | H | H |
| 7 | 3,4-dichlorobenzyl | H | H |
| 8 | 3-fluoro-4-trifluoromethylbenzyl | H | H |
| 9 | 4-fluoro-3-trifluoromethylbenzyl | H | H |
| 10 | 2,6-difluoro-4-methoxybenzyl | H | H |
| 11 | 2-fluoro-4-methoxybenzyl | H | H |
| 12 | 2-bromo-6-fluorobenzyl | H | H |
| 13 | 2-bromo-6-chlorobenzyl | H | H |
| 14 | 2-chloro-6-cyanobenzyl | H | H |
| 15 | 2-cyano-6-fluorobenzyl | H | H |
| 16 | 2-chloro-4-cyanobenzyl | H | H |
| 17 | 4-cyano-2-fluorobenzyl | H | H |
| 18 | 4-cyano-3-fluorobenzyl | H | H |
| 19 | 2-fluoro-6-methylbenzyl | H | H |
| 20 | 2,6-difluorobenzyl | CH₃ | H |
| 21 | 2-chloro-6-fluorobenzyl | CH₃ | H |
| 22 | 2,4-dichlorobenzyl | CH₃ | H |
| 23 | 2,4,6-trifluorobenzyl | CH₃ | H |
| 24 | 2,6-dichlorobenzyl | CH₃ | H |
| 25 | 3,4-difluorobenzyl | CH₃ | H |
| 26 | 3,4-dichlorobenzyl | CH₃ | H |
| 27 | 3-fluoro-4-trifluoromethylbenzyl | CH₃ | H |
| 28 | 4-fluoro-3-trifluoromethylbenzyl | CH₃ | H |
| 29 | 2,6-difluoro-4-methoxybenzyl | CH₃ | H |
| 30 | 2-fluoro-4-methoxybenzyl | CH₃ | H |
| 31 | 2-bromo-6-fluorobenzyl | CH₃ | H |
| 32 | 2-bromo-6-chlorobenzyl | CH₃ | H |
| 33 | 2-chloro-6-cyanobenzyl | CH₃ | H |
| 34 | 2-cyano-6-fluorobenzyl | CH₃ | H |
| 35 | 2-chloro-4-cyanobenzyl | CH₃ | H |
| 36 | 4-cyano-2-fluorobenzyl | CH₃ | H |
| 37 | 4-cyano-3-fluorobenzyl | CH₃ | H |
| 38 | 2-fluoro-6-methylbenzyl | CH₃ | H |
| 39 | 2,6-difluorobenzyl | H | CH₃ |
| 40 | 2-chloro-6-fluorobenzyl | H | CH₃ |
| 41 | 2,4-dichlorobenzyl | H | CH₃ |
| 42 | 2,4,6-trifluorobenzyl | H | CH₃ |
| 43 | 2,6-dichlorobenzyl | H | CH₃ |
| 44 | 3,4-difluorobenzyl | H | CH₃ |
| 45 | 3,4-dichlorobenzyl | H | CH₃ |
| 46 | 3-fluoro-4-trifluoromethylbenzyl | H | CH₃ |
| 47 | 4-fluoro-3-trifluoromethylbenzyl | H | CH₃ |
| 48 | 2,6-difluoro-4-methoxybenzyl | H | CH₃ |
| 49 | 2-fluoro-4-methoxybenzyl | H | CH₃ |
| 50 | 2-bromo-6-fluorobenzyl | H | CH₃ |
| 51 | 2-bromo-6-chlorobenzyl | H | CH₃ |
| 52 | 2-chloro-6-cyanobenzyl | H | CH₃ |
| 53 | 2-cyano-6-fluorobenzyl | H | CH₃ |
| 54 | 2-chloro-4-cyanobenzyl | H | CH₃ |
| 55 | 4-cyano-2-fluorobenzyl | H | CH₃ |
| 56 | 4-cyano-3-fluorobenzyl | H | CH₃ |
| 57 | 2-fluoro-6-methylbenzyl | H | CH₃ |
| 58 | 2,6-difluorobenzyl | CH₃ | CH₃ |
| 59 | 2-chloro-6-fluorobenzyl | CH₃ | CH₃ |
| 60 | 2,4-dichlorobenzyl | CH₃ | CH₃ |
| 61 | 2,4,6-trifluorobenzyl | CH₃ | CH₃ |
| 62 | 2,6-dichlorobenzyl | CH₃ | CH₃ |
| 63 | 3,4-difluorobenzyl | CH₃ | CH₃ |
| 64 | 3,4-dichlorobenzyl | CH₃ | CH₃ |
| 65 | 3-fluoro-4-trifluoromethylbenzyl | CH₃ | CH₃ |
| 66 | 4-fluoro-3-trifluoromethylbenzyl | CH₃ | CH₃ |
| 67 | 2,6-difluoro-4-methoxybenzyl | CH₃ | CH₃ |
| 68 | 2-fluoro-4-methoxybenzyl | CH₃ | CH₃ |
| 69 | 2-bromo-6-fluorobenzyl | CH₃ | CH₃ |
| 70 | 2-bromo-6-chlorobenzyl | CH₃ | CH₃ |
| 71 | 2-chloro-6-cyanobenzyl | CH₃ | CH₃ |
| 72 | 2-cyano-6-fluorobenzyl | CH₃ | CH₃ |
| 73 | 2-chloro-4-cyanobenzyl | CH₃ | CH₃ |
| 74 | 4-cyano-2-fluorobenzyl | CH₃ | CH₃ |
| 75 | 4-cyano-3-fluorobenzyl | CH₃ | CH₃ |
| 76 | 2-fluoro-6-methylbenzyl | CH₃ | CH₃ |

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfectly Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e.g. wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e.g. by glycosylation or polymer additions such as prenylated, acetylated or farnesylated moieties or PEG moieties.

The compounds I and compositions thereof, respectively, are particularly suitable for controlling the following plant diseases:

Albugo spp. (white rust) on ornamentals, vegetables (e.g. A. candida) and sunflowers (e.g. A. tragopogonis); Alternaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. alternata) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Elysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sofina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochllobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohllum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani on soybeans and F. verticilkides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grain-staining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or, rotbrenner', anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secails (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Eysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustllago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

The compounds I and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, coiling lubricants, fiber or fabrics, against the infestation and destruction by harmful microorganisms, such as fungi and bacteria.

The compounds I and compositions thereof, resepectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e.g. increased biomass and/or increased content of valuable ingredients), plant vigor (e.g. improved plant growth and/or greener leaves (“greening effect”)), quality (e.g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising a solvent or solid carrier and at least one compound I and to the use for controlling harmful fungi.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e.g. solutions, emulsions, suspensions, dusts, powders, pastes and granules. The composition type depends on the particular intended purpose; in each case, it should ensure a fine and uniform distribution of the compound according to the invention.

Examples for composition types are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF).

Usually the composition types (e.g. SC, OD, FS, EC, WG, SG, WP, SP, SS, WS, GF) are employed diluted. Composition types such as DP, DS, GR, FG, GG and MG are usually used undiluted.

The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4th Ed., McGraw-Hill, New York, 1963, S. 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and anorganic thickeners, bactericides, anti-freezing agents, anti-foaming agents, if appropriate colorants and tackifiers or binders (e.g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e.g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e.g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum).

Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. Application can be carried out before or during sowing. Methods for applying or treating agrochemical compounds and compositions thereof, respectively, on to plant propagation material, especially seeds, are known in the art, and include dressing, coating, pelleting, dusting, soaking and in-furrow application methods of the propagation material. In a preferred embodiment, the compounds or the compositions thereof, respectively, are applied on to the plant propagation material by a method such that germination is not induced, e.g. by seed dressing, pelleting, coating and dusting.

In a preferred embodiment, a suspension-type (FS) composition is used for seed treatment. Typcially, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water.

The active substances can be used as such or in the form of their compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the active substances according to the invention.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance.

The active substances may also be used successfully in the ultra-low-volume process (ULV), it being possible to apply compositions comprising over 95% by weight of active substance, or even to apply the active substance without additives.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are, e.g., 0.001 g to 2 kg, preferably 0.005 g to 1 kg, of active substance per cubic meter of treated material.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of active substances, in conjunction with which the compounds according to the invention can be used, is intended to illustrate the possible combinations but does not limit them:
A) Respiration inhibitors
   Inhibitors of complex III at Qₒ site (e.g. strobilurins): azoxystrobin, coumethoxystrobin, coumoxystrobin, dimoxystrobin, enestroburin, fenaminstrobin, fenoxystrobin/flufenoxystrobin, fluoxastrobin, kresoxim-methyl, metominostrobin, orysastrobin, picoxystrobin, pyraclostrobin, pyrametostrobin, pyraoxystrobin, trifloxystrobin, 2-[2-(2,5-dimethyl-phenoxymethyl)-phenyl]-3-methoxy-acrylic acid methyl ester and 2-(2-(3-(2,6-dichlorophenyl)-1-methyl-allylideneaminooxymethyl)-phenyl)-2-methoxyimino-N-methyl-acetamide, pyribencarb, triclopyricarb/chlorodincarb, famoxadone, fenamidone;
   inhibitors of complex III at Q, site: cyazofamid, amisulbrom;
   inhibitors of complex II (e.g. carboxamides): benodanil, bixafen, boscalid, carboxin, fenfuram, fluopyram, flutolanil, fluxapyroxad, furametpyr, isopyrazam, mepronil, α-ylcarboxin, penflufen, penthiopyrad, sedaxane, tecloftalam, thifluzamide, N-(4′-trifluoromethylthiobiphenyl-2-yl)-3-difluoromethyl-1-methyl-1H-pyrazole-4-carboxamide, N-(2-(1,3,3-trimethyl-butyl)-phenyl)-1,3-dimethyl-5-fluoro-1H-pyrazole-4-carboxamide and N-[9-(dichloromethylene)-1,2,3,4-tetrahydro-1,4-methanonaphthalen-5-yl]-3-(difluoromethyl)-1-methyl-1H-pyrazole-4-carboxamide (Hambra/SYN 192);
   other respiration inhibitors (e.g. complex I, uncouplers): diflumetorim; nitrophenyl derivates: binapacryl, dinobuton, dinocap, fluazinam; ferimzone; organometal compounds: fentin salts, such as fentin-acetate, fentin chloride or fentin hydroxide; ametoctradin; and silthiofam;
B) Sterol biosynthesis inhibitors (SBI fungicides)
   C14 demethylase inhibitors (DMI fungicides): triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole; imidazoles: imazalil, pefurazoate, prochloraz, triflumizol; pyrimidines, pyridines and piperazines: fenarimol, nuarimol, pyrifenox, triforine;
   Delta14-reductase inhibitors: aldimorph, dodemorph, dodemorph-acetate, fenpropimorph, tridemorph, fenpropidin, piperalin, spiroxamine;
   Inhibitors of 3-keto reductase: fenhexamid;
C) Nucleic acid synthesis inhibitors
   phenylamides or acyl amino acid fungicides: benalaxyl, benalaxyl-M, kiralaxyl, metalaxyl, metalaxyl-M (mefenoxam), ofurace, oxadixyl;
   others: hymexazole, octhilinone, oxolinic acid, bupirimate;
D) Inhibitors of cell division and cytoskeleton
   tubulin inhibitors, such as benzimidazoles, thiophanates: benomyl, carbendazim, fuberidazole, thiabendazole, thiophanate-methyl; triazolopyrimidines: 5-chloro-7-(4-methylpiperidin-1-yl)-6-(2,4,6-trifluorophenyl)-[1,2,4]triazolo[1,5-a]pyrimidine
   other cell division inhibitors: diethofencarb, ethaboxam, pencycuron, fluopicolide, zoxamide, metrafenone, pyriofenone;
E) Inhibitors of amino acid and protein synthesis
   methionine synthesis inhibitors (anilino-pyrimidines): cyprodinil, mepanipyrim, pyrimethanil;
   protein synthesis inhibitors: blasticidin-S, kasugamycin, kasugamycin hydrochloride-hydrate, mildiomycin, streptomycin, oxytetracyclin, polyoxine, validamycin A;
F) Signal transduction inhibitors
   MAP/histidine kinase inhibitors: fluoroimid, iprodione, procymidone, vinclozolin, fenpiclonil, fludioxonil;
   G protein inhibitors: quinoxyfen;
G) Lipid and membrane synthesis inhibitors
   Phospholipid biosynthesis inhibitors: edifenphos, iprobenfos, pyrazophos, isoprothiolane;
   lipid peroxidation: dicloran, quintozene, tecnazene, tolclofos-methyl, biphenyl, chloroneb, etridiazole;
   phospholipid biosynthesis and cell wall deposition: dimethomorph, flumorph, mandipropamid, pyrimorph, benthiavalicarb, iprovalicarb, valifenalate and N-(1-(1-(4-cyano-phenyl)ethanesulfonyl)-but-2-yl) carbamic acid-(4-fluorophenyl) ester;
   compounds affecting cell membrane permeability and fatty acides: propamocarb, propamocarb-hydrochlorid
H) Inhibitors with Multi Site Action
   inorganic active substances: Bordeaux mixture, copper acetate, copper hydroxide, copper oxychloride, basic copper sulfate, sulfur;
   thio- and dithiocarbamates: ferbam, mancozeb, maneb, metam, metiram, propineb, thiram, zineb, ziram;
   organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonannide;
   guanidines and others: guanidine, dodine, dodine free base, guazatine, guazatineacetate, iminoctadine, iminoctadine-triacetate, iminoctadine-tris(albesilate), dithianon;
I) Cell wall synthesis inhibitors
   inhibitors of glucan synthesis: validamycin, polyoxin B; melanin synthesis inhibitors: pyroquilon, tricyclazole, carpropamid, dicyclomet, fenoxanil;
J) Plant defence inducers
   acibenzolar-5-methyl, probenazole, isotianil, tiadinil, prohexadione-calcium; phosphonates: fosetyl, fosetyl-aluminum, phosphorous acid and its salts;
K) Unknown mode of action
   bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, methoxy-acetic acid 6-tert-butyl-8-fluoro-2,3-dimethyl-quinolin-4-yl ester, N-Methyl-2-{1-[(5-methyl-3-trifluoromethyl-1H-pyrazol-1-O-acetyl]-piperidin-4-yl}-N-[(1R)-1,2,3,4-tetrahydronaphthalen-1-yl]-4-thiazolecarboxamide, 3-[5-(4-methylphenyl)-2,3-dimethylisoxazolidin-3-yl]-pyridine, 3-[5-(4-chloro-phenyl)-2,3-dimethyl-isoxazolidin-3-yl]-pyridine (pyrisoxazole), N-(6-methoxy-pyridin-3-yl)cyclopropanecarboxylic acid amide, 5-chloro-1-(4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxyacetamide;

L) Antifungal biocontrol agents, plant bioactivators: Ampelomyces quisqualis (e.g. AQ 10® from Intrachem Bio GmbH & Co. KG, Germany), Aspergillus flavus (e.g. AFLAGUARD® from Syngenta, CH), Aureobasidium pullulans (e.g. BOTECTOR® from bio-ferm GmbH, Germany), Bacillus pumilus (e.g. NRRL Accession No. B-30087 in SONATA® and BALLAD® Plus from AgraQuest Inc., USA), Bacillus subtilis (e.g. isolate NRRL-Nr. B-21661 in RHAPSODY®, SERENADE® MAX and SERENADE® ASO from AgraQuest Inc., USA), Bacillus subtilis var. amylolique-faciens FZB24 (e.g. TAEGRO® from Novozyme Biologicals, Inc., USA), Candida oleophlia I-82 (e.g. ASPIRE® from Ecogen Inc., USA), Candida saitoana (e.g. BIOCURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans (e.g. CONTANS® from Prophyta, Germany), Cryphonectria parasitica (e.g. Endothia parasitica from CNICM, France), Cryptococcus albidus (e.g. YIELD PLUS® from Anchor Bio-Technologies, South Africa), Fusarium oxysporum (e.g. BIOFOX® from S.I.A.P.A., Italy, FUSACLEAN® from Natural Plant Protection, France), Metschnikowia fructicola (e.g. SHEMER® from Agrogreen, Israel), Microdochium dimerum (e.g. ANTIBOT® from Agrauxine, France), Phlebiopsis gigantea (e.g. ROTSOP® from Verdera, Finland), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis (e.g. REGALIA® from Marrone BioInnovations, USA), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. atroviride LC52 (e.g. SENTINEL® from Agrimm Technologies Ltd, NZ), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB BioInnovation AB, Sweden), T. stromaticum (e.g. TRICOVAB® from C.E.P.L.A.C., Brazil), T. virens GL-21 (e.g. SOILGARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy), Ulocladium oudemansii HRU3 (e.g. BOTRY-ZEN® from Botry-Zen Ltd, NZ);

The present invention furthermore relates to agrochemical compositions comprising a mixture of at least one compound I (component 1) and at least one further active substance useful for plant protection, e.g. selected from the groups A) to L) (component 2), in particular one further fungicide, e.g. one or more fungicide from the groups A) to F), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to L), as described above, is more efficient than combating those fungi with individual compounds I or individual fungicides from groups A) to L). By applying compounds I together with at least one active substance from groups A) to L) a synergistic effect can be obtained, i.e. more then simple addition of the individual effects is obtained (synergistic mixtures).

In binary mixtures, i.e. compositions according to the invention comprising one compound I (component 1) and one further active substance (component 2), e.g. one active substance from groups A) to O), the weight ratio of component 1 and component 2 generally depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:3 to 3:1.

In ternary mixtures, i.e. compositions according to the invention comprising one compound I (component 1) and a first further active substance (component 2) and a second further active substance (component 3), e.g. two active substances from groups A) to L), the weight ratio of component 1 and component 2 depends from the properties of the active substances used, preferably it is in the range of from 1:50 to 50:1 and particularly in the range of from 1:10 to 10:1, and the weight ratio of component 1 and component 3 preferably is in the range of from 1:50 to 50:1 and particularly in the range of from 1:10 to 10:1.

According to one embodiment, individual components of the composition according to the invention such as parts of a kit or parts of a binary or ternary mixture may be mixed by the user himself in a spray tank and further auxiliaries may be added, if appropriate (tank mix).

Preference is also given to mixtures comprising a compound I (component 1) and at least one active substance selected from group A) (component 2) and particularly selected from azoxystrobin, dimoxystrobin, fluoxastrobin, kresoxim-methyl, orysastrobin, picoxystrobin, pyraclostrobin, trifloxystrobin; famoxadone, fenamidone; bixafen, boscalid, fluopyram, fluxapyroxad, isopyrazam, penflufen, penthiopyrad, sedaxane; ametoctradin, cyazofamid, fluazinam, fentin salts, such as fentin acetate.

Preference is given to mixtures comprising a compound of formula I (component 1) and at least one active substance selected from group B) (component 2) and particularly selected from cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, fenarimol, triforine; dodemorph, fenpropimorph, tridemorph, fenpropidin, spiroxamine; fenhexamid.

Preference is given to mixtures comprising a compound of formula I (component 1) and at least one active substance selected from group C) (component 2) and particularly selected from metalaxyl, (metalaxyl-M) mefenoxam, ofurace.

Preference is given to mixtures comprising a compound of formula I (component 1) and at least one active substance selected from group D) (component 2) and particularly selected from benomyl, carbendazim, thiophanate-methyl, ethaboxam, fluopicolide, zoxamide, metrafenone, pyriofenone.

Preference is also given to mixtures comprising a compound I (component 1) and at least one active substance selected from group E) (component 2) and particularly selected from cyprodinil, mepanipyrim, pyrimethanil.

Preference is also given to mixtures comprising a compound I (component 1) and at least one active substance selected from group F) (component 2) and particularly selected from iprodione, fludioxonil, vinclozolin, quinoxyfen.

Preference is also given to mixtures comprising a compound I (component 1) and at least one active substance selected from group G) (component 2) and particularly selected from dimethomorph, flumorph, iprovalicarb, benthiavalicarb, mandipropamid, propamocarb.

Preference is also given to mixtures comprising a compound I (component 1) and at least one active substance selected from group H) (component 2) and particularly selected from copper acetate, copper hydroxide, copper oxychloride, copper sulfate, sulfur, mancozeb, metiram, propineb, thiram, captafol, folpet, chlorothalonil, dichlofluanid, dithianon.

Preference is also given to mixtures comprising a compound I (component 1) and at least one active substance selected from group I) (component 2) and particularly selected from carpropamid and fenoxanil.

Preference is also given to mixtures comprising a compound I (component 1) and at least one active substance selected from group J) (component 2) and particularly selected from acibenzolar-5-methyl, probenazole, tiadinil, fosetyl, fosetyl-aluminium, H₃PO₃ and salts thereof.

Preference is also given to mixtures comprising a compound I (component 1) and at least one active substance selected from group K) (component 2) and particularly selected from cymoxanil, proquinazid and N-methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide.

The mixtures of active substances can be prepared as compositions comprising besides the active ingredients at least one inert ingredient by usual means, e.g. by the means given for the compositions of compounds I.

Concerning usual ingredients of such compositions reference is made to the explanations given for the compositions containing compounds I.

### SYNTHESIS EXAMPLES

With appropriate modification of the starting materials, the procedures given in the synthesis examples below were used to obtain further compounds I. The compounds produced in this manner are listed in Table I-A below. The corresponding physical data can be found in Table I-B.

### Example 1

### N-Ethyl-N′-{1-[2-rnethoxyimino-3-(3-trifluoromethyl-phenoxy)-propyl]-6-methyl-1H-benzoimidazol-5-yl}-N-methyl-formamidine

### 1a) 1-Amino-3-(3-trifluoromethyl-phenoxy)-propan-2-one O-methyl-oxime

A solution of 10.70 g (37.1 mmol) 1-Azido-3-(3-trifluoromethyl-phenoxy)-propan-2-on O-methyl-oxime in 750 ml methanol (MeOH) was hydrated with an H-Cube™ (10% Pd on charcoal, 30 mm cartouche, 30° C., 30 bar H₂ pressure, flow rate: 1 ml/min). After evaporation of the solvent in vacuo 9.50 g of the desired product have been obtained to be used without further purification.

### 1b) 1-(5-Methyl-2,4-dinitro-phenylamino)-3-(3-trifluoromethyl-phenoxy)-propan-2-on O-methyl-oxime

To a solution of 7.00 g (35.0 mmol) 1-fluoro-5-methyl-2,4-dinitro-benzene in 100 ml MeOH have been added with stirring at about 25° C. first 3.55 g (35.0 mmol) triethylamine and subsequently a solution of 9.00 g 1-amino-3-(3-trifluoromethyl-phenoxy)-propan-2-one O-methyl-oxime, (34.3 mmol) in 100 ml MeOH. The reaction was stirred for about 12 h at about 25° C., and a yellow precipitate has been observed. The reaction mixture has been concentrated in vacuo and diluted with MTBE. After filtration, washing with n-pentane and evaporation of the solvent in vacuo 5.40 g of the desired product have been obtained.

### 1c) 1-(2-Amino-5-methyl-4-nitro-phenylamino)-3-(3-trifluoromethyl-phenoxy)-propan-2-on O-methyl-oxime

A solution of 0.36 g (1.0 mmol) 1-(5-methyl-2,4-dinitro-phenylamino)-3-(3-trifluoromethyl-phenoxy)-propan-2-one O-methyl-oxime in 40 ml THF was hydrated with an H-Cube™ (10% Pd on charcoal, 30 mm cartouche, 25° C., 10 bar H₂ pressure, low rate 1 ml/min). The solvent was evaporated in vacuo and the product (0.30 g) was used without further purification.

### 1d) N-Ethyl-N′-{2-[2-methoxyimino-3-(3-trifluoromethyl-phenoxy)-propylamino]-4-methyl-5-nitro-phenyl}-N-methyl-formamidine

A solution of 2.30 g (5.6 mmol) 1-(2-amino-5-methyl-4-nitro-phenylamino)-3-(3-trifluoromethyl-phenoxy)-propan-2-one O-methyl-oxime and 3.00 g (22.5 mmol) N,N-ethyl-methylformamide dimethylacetal in 50 ml methylenechloride was stirred for about 12 h at about 25° C. After evaporation of the solvent in vacuo the redidue was purified by chromatography on silica with cyclohexane (Cy)/ethyl acetate (EtAc) to obtain 1.50 g of the desired product.

### 1e) 1-(6-Methyl-5-nitro-benzoimidazol-1-yl)-3-(3-trifluoromethyl-phenoxy)-propan-2-on O-methyl-oxime

A solution of 0.30 g (0.6 mmol) N-ethyl-N′-{2-[2-nnethoxyimino-3-(3-trifluoromethylphenoxy)-propylamino]-4-methyl-5-nitro-phenyl}-N-methyl-formamidine and 0.20 g (1.8 mmol) TFA in 20 ml THF was stirred for about 72 h at about 25° C. The solvent was evaporated in vacuo and the product (0.20 g) was used without further purification.

### 1f) 1-(5-Amino-6-methyl-benzoimidazol-1-yl)-3-(3-trifluoromethyl-phenoxy)-propan-2-one O-methyl-oxime

A solution of 0.5 g (1.2 mmol) 1-(6-methyl-5-nitro-benzoimidazol-1-yl)-3-(3-trifluoromethyl-phenoxy)-propan-2-one O-methyl-oxime in 30 ml MeOH was hydrated with an H-Cube™ (10% Pd on charcoal, 30 mm cartouche, 50° C., 50 bar H₂ pressure, low rate 1 ml/min). The solvent was evaporated in vacuo and the product (0.35 g) was used without further purification.

### 1g) N-Ethyl-N′-{1-[2-methoxyimino-3-(3-trifluoromethyl-phenoxy)-propyl]-6-methyl-1H-benzoimidazol-5-yl}-N-methyl-formamidine

A solution of 0.30 g (0.7 mmol) 1-(5-amino-6-methyl-benzoimidazol-1-yl)-3-(3-trifluoromethyl-phenoxy)-propan-2-one O-methyl-oxime and 0.20 g (1.5 mmol) N,N-ethylmethylformamid-dimethyl acetal in 20 ml methylene chloride (DCM) was stirred for about 12 h at about 25° C. After evaporation of the solvent in vacuo the residue was purified by chromatography on silica (Cy/EtAc) to obtain 0.24 g of the desired product.

### Example 2

### N-Ethyl-N′-{1-[2-methoxyimino-3-(3-trifluoromethyl-phenoxy)-propyl]-1H-benzoimidazol-5-yl}-N-methyl-formamidine

### 2a) 1-(5-Nitro-benzoimidazol-1-yl)-3-(3-trifluoromethyl-phenoxy)-propan-2-on O-methyloxime and 1-(6-Nitro-benzoimidazol-1-yl)-3-(3-trifluoromethyl-phenoxy)-propan-2-on O-methyl-oxime

A solution of 0.65 g (4.0 mmol) 5-nitrobenzimidazole in 10 ml DMF was stirred with 1.66 g (12.0 mmol) K₂CO₃ for about 10 min at about 25° C. Then, 1.30 g (4.0 mmol) 1-bromo-3-(3-trifluoromethyl-phenoxy)-propan-2-one O-methyl-oxime have been added. Stirring was continued for about 12 h at about 25° C. Water was added and the mixture was extracted several times with MTBE. The organic layer was washed with water and saturated NaCl solution, dried with MgSO₄, the solvent was removed in vacuo and the residue has been purified by chromatography on silica (DCM/MeOH) to obtain 1.5 g of the mixture of product isomers.

### 2b) 1-(5-Amino-benzoimidazol-1-yl)-3-(3-trifluoromethyl-phenoxy)-propan-2-on O-methyl-oxime and 1-(6-Amino-benzoimidazol-1-yl)-3-(3-trifluoromethyl-phenoxy)-propan-2-on O-methyl-oxime

A solution of 1.70 g (4.2 mmol) of the product mixture obtained in 2a) in 150 ml MeOH was hydrogenated with an H-Cube™ (10% Pd on charcoal, 30 mm cartouche, 40° C., full-H H₂ pressure, flow rate: 1 ml/min). The solvent was removed in vacuo and the residue has been purified by chromatography on silica (DCM/MeOH) to obtain 1-(5-Amino-benzoimidazol-1-yl)-3-(3-trifluoromethyl-phenoxy)-propan-2-on O-methyl-oxime (0.25 g) and 1-(6-Amino-benzoimidazol-1-yl)-3-(3-trifluoromethylphenoxy)-propan-2-on O-methyl-oxime (0.30 g).

### 2c) N-Ethyl-N′-{1-[2-methoxyimino-3-(3-trifluoromethyl-phenoxy)-propyl]-1H-benzoimidazol-5-yl}-N-methyl-formamidine

A solution of 0.30 g (0.8 mmol) 1-(5-amino-benzoimidazol-1-yl)-3-(3-trifluoromethylphenoxy)-propan-2-one O-methyl-oxime and 0.21 g (1.6 mmol) N,N-ethyl-methylformamide dimethylacetal in 20 ml THF was stirred for about 12 h at about 25° C. The solvent was removed in vacuo and the residue has been purified by chromatography on silica (DCM/MeOH) to obtain 0.20 g of the desired product.

### Example 3

### N-Ethyl-N′-{1-[2-methoxyimino-3-(3-trifluoromethyl-phenoxy)-propyl]-2-trifluoromethyl-1H-benzoimidazol-5-yl}-N-methyl-formamidine and N-ethyl-N′-{3-[2-methoxyimino-3-(3-trifluoromethyl-phenoxy)-propyl]-2-trifluoromethyl-3H-benzoimidazol-5-yl}-N-methyl-formamidine

### 3a) N-Ethyl-N-methyl-N′-(2-trifluoromethyl-1H-benzoimidazol-5-yl)-formamidine

A solution of 1.01 g (5.0 mmol) 2-trifluoromethyl-1H-benzoimidazol-5-ylannine and 1.01 g (8.0 mmol) N,N-ethyl-methylformamide dimethylacetal in 20 ml THF was stirred for about 12 h at about 25° C. The solvent was removed in vacuo and the residue was purified by chromatography on silica (DCM/MeOH) to obtain 0.72 g of the desired product.

### 3b) N-Ethyl-N′-{1-[2-methoxyimino-3-(3-trifluoromethyl-phenoxy)-propyl]-2-trifluoromethyl-1H-benzoimidazol-5-yl}-N-methyl-formamidine and N-ethyl-N′-{3-[2-methoxyimino-3-(3-trifluoromethyl-phenoxy)-propyl]-2-trifluoromethyl-3H-benzoimidazol-5-yl}-N-methyl-formamidine

A solution of 0.34 g (1.3 mmol) N-ethyl-N-methyl-N′-(2-trifluoromethyl-1H-benzoimidazol-5-yl)-formamidine in 6 ml DMF was stirred with 0.52 g (3.8 mmol) K₂CO₃ for 10 min at about 25° C. Then 0.41 g (1.3 mmol) 1-bromo-3-(3-trifluoromethyl-phenoxy)-propan-2-one O-methyl-oxime have been added. Stirring was continued for about 12 h at about 25° C. Water was added. The mixture was extracted several times with MTBE. The organic layer was washed with water and concentrated NaCl solution, dried with MgSO₄ and the solvent removed in vacuo. The isomers contained in the crude product have been separated by chromatography on silica (DCM/MeOH). 0.15 g (N-ethyl-N′-{1-[2-methoxyimino-3-(3-trifluoromethyl-phenoxy)-propyl]-2-trifluoromethyl-1H-benzoimidazol-5-yl}-N-methyl-formamidine) and some N-ethyl-N′-{3-[2-methoxyimino-3-(3-trifluoromethyl-phenoxy)-propyl]-2-trifluoromethyl-3H-benzoimidazol-5-yl}-N-methyl-formamidine have been obtained.

### Example 4

### N-Ethyl-N′-{1-[2-methoxyimino-3-(3-trifluoromethyl-phenoxy)-propyl]-2,3-dihydro-1H-indol-5-yl}-N-methyl-formamidine

### 4a) 1-(5-Nitro-2,3-dihydro-indol-1-yl)-3-(3-trifluoromethyl-phenoxy)-propan-2-one O-methyl-oxime

A solution of 0.50 g (3.0 mmol) 5-nitro-2,3-dihydro-1H-indole in 10 ml DMF was stirred with 1.03 g (9.0 mmol) K₂CO₃ for 10 min at about 25° C. Then 1.00 g (0.3 mmol) 1-bromo-3-(3-trifluoromethyl-phenoxy)-propan-2-one O-methyl-oxime have been added. Stirring was continued for about 12 h at about 25° C. Water was added. The mixture was extracted several times with EtAc. The organic layer was washed with water and concentrated NaCl solution, dried with MgSO₄ and the solvent removed in vacuo. The residue was purified by chromatography on silica (Cy/EtAc) to obtain 0.90 g of the desired product.

### 4b) 1-(5-Amino-2,3-dihydro-indol-1-yl)-3-(3-trifluoromethyl-phenoxy)-propan-2-one O-methyl-oxime

A solution of 0.90 g (2.2 mmol) 1-(5-nitro-2,3-dihydro-indol-1-yl)-3-(3-trifluoromethylphenoxy)-propan-2-one O-methyl-oxime in 50 ml MeOH was hydrated with an H-Cube™ (10% Pd on charcoal, 30 mm cartouche, 40° C., full-H H₂ pressure, flow rate: 1 ml/min). After removal of the solvent in vacuo 0.8 g of crude product have been obtained to be used without further delay.

### 4c) N-Ethyl-N′-{1-[2-nnethoxyimino-3-(3-trifluoromethyl-phenoxy)-propyl]-2,3-dihydro-1H-indol-5-yl}-N-methyl-formamidine

A solution of 0.40 g (1.1 mmol) 1-(5-amino-2,3-dihydro-indol-1-yl)-3-(3-trifluoromethyl-phenoxy)-propan-2-one O-methyl-oxime and 0.28 g (2.1 mmol) N,N-ethyl-methylformamide dimethylacetal in 10 ml DCM have been stirred for about 72 h at about 25° C. The solvent has been removed in vacuo. The residue was purified by chromatography on silica (Cy/EtAc) to obtain 0.27 g of the desired product.

### Example 5

### N-Ethyl-N′-{1-[2-methoxyimino-3-(3-trifluoromethyl-phenoxy)-propyl]-2-methyl-2,3-dihydro-1H-indol-5-yl}-N-methyl-formamidine

### 5a) 2-Methyl-2,3-dihydro-1H-indol-5-ylamine

A solution of 4.50 g (25.3 mmol) 2-methyl-5-nitro-2,3-dihydro-1H-indole in 500 ml MeOH was hydrated with an H-Cube™ (10% Pd on charcoal, 55 mm cartouche, 40° C., hull-H H₂ pressure, flow rate 1 ml/nnin). The solvent was removed in vacuo. The residue was purified by chromatography on silica (Cy/EtAc) to obtain 1.6 g of the desired product.

### 5b) N-Ethyl-N-methyl-N′-(2-methyl-2,3-dihydro-1H-indol-5-yl)-formamidine

A solution of 1.40 g (9.4 mmol) 2-methyl-2,3-dihydro-1H-indol-5-ylamine and 2.52 g (18.9 mmol) N,N-ethyl-methylformamide dimethylacetal in 15 ml DCM were stirred for about 72 h at about 25° C. The solvent was removed in vacuo. The residue was purified by chromatography on silica (DCM/MeOH) to obtain 1.50 g of the desired product.

### 5c) N-Ethyl-N′-{1-[2-methoxyimino-3-(3-trifluoromethyl-phenoxy)-propyl]-2-methyl-2,3-dihydro-1H-indol-5-yl}-N-methyl-formamidine

A solution of 0.20 g (0.9 mmol) N-ethyl-N-methyl-N′-(2-methyl-2,3-dihydro-1H-indol-5-yl)-formamidine in 10 ml DMF was stirred with 0.20 g (1.8 mmol) K₂CO₃ and a catalytical amount of KI for 10 min at about 25° C. Then 0.30 g (0.3 mmol) 1-bromo-3-(3-trifluoromethyl-phenoxy)-propan-2-one O-methyl-oxime have been added. Stirring was continued for about 12 h at about 25° C. Water was added. The mixture was extracted several times with EtAc. The organic layer was washed with water and conc. NaCl solution, dried with MgSO₄ and the solvent removed in vacuo. The residue was purified by chromatography on silica (Cy/EtAc) to obtain 0.32 g of the desired product.

### Example 6

### N-Ethyl-N′-{1-[2-methoxyimino-3-(3-trifluoromethyl-phenoxy)-propyl]-6-methyl-1H-benzotriazol-5-yl}-N-methyl-formamidine

### 6a) 1-(6-Methyl-5-nitro-benzotriazol-1-yl)-3-(3-trifluoromethyl-phenoxy)-propan-2-on O-methyl-oxime

To a solution of 5.80 g (15.2 mmol) 1-(2-amino-5-methyl-4-nitro-phenylamino)-3-(3-trifluoromethyl-phenoxy)-propan-2-one O-methyl-oxime in a mixture of 50 ml water, 20 ml acetic acid and 28 ml acetonitrile (AcN) was added dropwise with stirring at about 25° C. a solution of 1.05 g (15.2 mmol) sodium nitrite in 30 ml water. Stirring was continued for about 12 h at about 25° C. Then saturated NaCl solution was added, and the product was extracted with EtAc. The combined extracts have been washed with saturated NaHCO₃-solution and with saturated NaCl solution, dried with MgSO₄ and the solvent removed in vacuo. The residue was purified by chromatography on silica (Cy/EtAc) to obtain 1.20 g of the desired product.

### 6b) 1-(5-Amino-6-methyl-benzotriazol-1-yl)-3-(3-trifluoromethyl-phenoxy)-propan-2-on O-methyl-oxime

A solution of 1.70 g (4.2 mmol) 1-(6-methyl-5-nitro-benzotriazol-1-yl)-3-(3-trifluoromethyl-phenoxy)-propan-2-one O-methyl-oxime in 100 ml MeOH was hydrated with an H-Cube™ (10% Pd on charcoal, 30 mm cartouche, 50° C., 50 bar H₂ pressure, flow rate: 1 ml/min). After removal of the solvent in vacuo 1.2 g of crude product have been obtained to be used without further purification.

### 6c) N-Ethyl-N′-{1-[2-methoxyimino-3-(3-trifluoromethyl-phenoxy)-propyl]-6-methyl-1H-benzotriazol-5-yl}-N-methyl-formamidine

To a mixture of 1.50 g (4.0 mmol) 1-(5-amino-6-methyl-benzotriazol-1-yl)-3-(3-trifluoromethyl-phenoxy)-propan-2-one O-methyl-oxime and 18 ml trimethoxymethane a catalytical amount of p-toluic acid monhoydrate was added and the reaction was heated for 30 min to about 100° C. Then, the volatile components have been removed in vacuo and the residue was dissolved in 10 ml THF. After addition of 0.71 g (11.9 mmol) ethylmethylannine the mixture was heated for 2 h to about 100° C. Then the volatile components have been removed in vacuo, the residue was diluted with water and extracted with EtAc. The organic layer was washed with water and conc. NaCl solution, dried with MgSO₄ and the solvent removed in vacuo. The residue was purified by chromatography on silica (Cy/EtAc) to obtain 0.12 g of the desired product.

### Example 7

### N-Ethyl-N′-{1-[2-methoxyimino-3-(3-trifluoromethyl-phenoxy)-propyl]-2,3-dioxo-2,3-dihydro-1H-indol-5-yl}-N-methyl-formamidine

### 7a) 1-[2-Methoxyimino-3-(3-trifluoromethyl-phenoxy)-propyl]-5-nitro-1H-indol-2,3-dione

A solution of 1.50 g (7.8 mmol) 5-nitro-1H-indole-2,3-dione in 20 ml N,N-dimethylformamide was stirred with 3.20 g (28.4 mmol) K₂CO₃ for 10 min at about 25° C. Then a solution of 2.54 g (7.8 mmol) 1-bromo-3-(3-trifluoromethyl-phenoxy)-propan-2-one O-methyl-oxime in 5 ml DMF was added. Stirring was continued for about 12 h at about 25° C. Water was added. The mixture was extracted with EtAc. The organic layer was washed with water and concentrated NaCl solution, dried with MgSO₄ and the solvent removed in vacuo. The residue was purified by chromatography on silica (Cy/EtAc) to obtain 1.30 g of the desired product.

### 7b) 5-Amino-1-[2-methoxyimino-3-(3-trifluoromethyl-phenoxy)-propyl]-1H-indol-2,3-dione

A solution of 1.80 g (4.1 mmol) 1-[2-methoxyimino-3-(3-trifluoromethyl-phenoxy)-propyl]-5-nitro-1H-indol-2,3-dione in 100 ml THF was hydrated with an H-Cube™ (10% Pd on charcoal, 30 mm cartouche, 50° C., 50 bar H₂ pressure, flow rate: 1 ml/min). After removal of the solvent in vacuo 0.8 g of crude product have been obtained to be used without further purification.

### 7c) N-Ethyl-N′-{1-[2-methoxyimino-3-(3-trifluoromethyl-phenoxy)-propyl]-2,3-dioxo-2,3-dihydro-1H-indol-5-yl}-N-methyl-formamidine

A solution of 1.30 g (3.2 mmol) 5-amino-1-[2-methoxyimino-3-(3-trifluoromethylphenoxy)-propyl]-1H-indol-2,3-dione and 0.85 g (6.4 mmol) N,N-ethyl-methylformamide dimethylacetal in 20 ml DCM was stirred for about 12 h at about 25° C. The solvent was removed in vacuo. The residue was purified by chromatography on silica (DCM/MeOH to obtain 0.36 g of the desired product.

### Example 8

### N-Ethyl-N′-{1-[2-nnethoxyimino-3-(3-trifluoromethyl-phenoxy)-propyl]-6-methyl-1H-indazol-5-yl}-N-methyl-formamidine

### 8a) N-Ethyl-N-methyl-N′-(6-methyl-1H-indazol-5-yl)-formamidine

A solution of 0.50 g (3.4 mmol) 6-methyl-1H-indazol-5-ylamine and 0.91 g (6.8 mmol) N,N-ethyl-methylformamide dimethylacetal in 10 ml THF was stirred for about 12 h at about 25° C. Then the volatile components have been removed in vacuo and the residue has been dissolved in EtAc. The organic layer was washed with water and conc. NaCl solution, dried with MgSO₄ and the solvent removed in vacuo to obtain 0.50 g of the desired product.

### 8b) N-Ethyl-N′-{1-[2-methoxyimino-3-(3-trifluoromethyl-phenoxy)-propyl]-6-methyl-1H-indazol-5-yl}-N-methyl-formamidine

A solution of 0.25 g (1.2 mmol) N-ethyl-N-methyl-N′-(6-methyl-1H-indazol-5-yl)-formamidine in 4 ml DMF was stirred with 0.32 g (2.3 mmol) K₂CO₃ for 15 min at about 25° C. Then a solution of 0.38 g (1.2 mmol) 1-bromo-3-(3-trifluoromethyl-phenoxy)-propan-2-one O-methyl-oxime in 2 ml DMF has been added. Stirring was continued for about 12 h at about 25° C. The mixture was diluted with water and extracted with MTBE. The organic layer was washed with water and conc. NaCl solution, dried with MgSO₄ and the solvent removed in vacuo. The residuehas been purified by chromatography on silica (DCM/MeOH) to obtain 0.27 g of the desired product.

In some reactions the alkylation did also yield some product alkylated on the other nitrogen in the indazole ring.

### Example 9

### N′-(1-{3-(2-Fluoro-phenoxy)-2-methoxyimino-propyl}-1H-indol-5-yl)-N,N-dimethyl-formamidine

### 9a) N′-(1H-Indol-5-yl)-N,N-dimethylformamidine

A mixture of 5-aminoindole (2.5 g, 19 mmol) and N,N-dimethylformamide dimethylacetal (3.7 ml, 28 mmol) was heated at about 70° C. for 30 min under neat conditions. TLC revealed clean conversion. The reaction mixture was concentrated. The residue was triturated with hexane (3×) to obtain 3.5 g of the desired product as solid.

### 9b) N′-(1-{3-Chloro-2-[nnethoxyimino]-propyl}-1H-indol-5-yl)-N,N-dimethyl-formamidine

To a solution of N′-(1H-indol-5-yl)-N,N-dimethylformamidine (3.5 g, 18.5 mmol) in dry DMSO (22 ml) was added KOtBu (2.3 g, 20.5 mmol) and it was allowed to stir for about 1 h at about 25° C. This solution was then added to a solution of 1,3-dichloropropan-2-one O-methyloxime (5.7 g, 37 mmol) in DMSO (44 ml) at 0° C. and stirred for about 5 h at about 25° C. The reaction mixture was poured into ice water and extracted with MTBE (3×). The combined organic layers were dried (Na₂SO₄) and concentrated. Column purification (100-200 silica gel, 40% EtAc/hexane with 1-2 drops of triethylamine (TEA) per 100 ml of eluent) provided 2.7 g of the desired product as sticky liquid.

### 9c) N′-(1-{3-(2-Fluoro-phenoxy)-2-methoxyimino-propyl}-1H-indol-5-yl)-N,N-dimethylformamidine

To a solution of 2-fluorophenol (303 mg, 2.6 mmol) in DMSO (2.4 ml) was added KOtBu (303 mg, 2.6 mol) at about 25° C. and stirred for 1 h. This solution was added to a solution of N′-(1-{3-chloro-2-[methoxy-imino]-propyl}-1H-indol-5-yl)-N,N-dimethylformamidine (750 mg, 2.4 mmol) in DMSO (1.2 ml) and allowed to stir at about 25° C. for about 12 h. LC-MS revealed clean conversion. The reaction mixture was poured into ice water and extracted with MTBE (3×). The combined organic layers were dried (Na₂SO₄) and column purification (70-230 mesh basic alumina, 8-10% EtAc/hexane) provided 300 mg the desired compound as white sticky material.

### Example 10

### N′-{1-[2-Methoxyimino-3-(4-methoxy-phenoxy)-propyl]-1H-indol-5-yl}-N,N-dimethyl-formamidine

A suspension of N′-(1-{3-chloro-2-[methoxy-imino]-propyl}-1H-indol-5-yl)-N,N-dimethyl-formamidine (750 mg, 2.4 mmol), 4-methoxy aniline (332 mg, 2.6 mmol) and Na₂CO₃ (1.1 g, 9.8 mmol) in acetonitrile (12 ml) was refluxed for about 24 h. LC-MS revealed completion of reaction. EtAc was added and the organic layer was washed with water, brine and dried (Na₂SO₄). Concentration and column purification (70-230 mesh basic alumina, 10% EtAc/hexane) provided 150 mg of the desired compound as light brown solid.

### Example 11

### 5-(Dimethylamino-methyleneamino)-1-[2-methoxyimino-3-(3-trifluoromethyl-phenoxy)-propyl]-1H-indole-3-carboxylic acid amide

### 11a) 5-(Dimethylamino-methyleneamino)-1H-indole-3-carboxylic acid amide

A mixture of 5-amino-1H-indole-3-carboxylic acid amide (1.7 g, 9.7 mmol) and N,N-dimethylformamide dimethylacetal (1.6 ml, 10.6 mmol) was heated at about 60° C. for 30 min under neat conditions. TLC revealed clean conversion. The reaction mixture was concentrated and the resulting residue was triturated with hexane (3×) to obtain 2.5 g of the desired product as grey solid.

### 11b) 1-{3-Chloro-2-[methoxyimino]-propyl}-5-(dimethylannino-methylene-amino)-1H-indole-3-carboxylic acid amide

To a solution of 5-(dimethylamino-methyleneamino)-1H-indole-3-carboxylic acid amide (2.5 g, 10.8 mmol) in 26 ml dry DMSO was added KOtBu (1.2 g, 10.8 mmol) and it was allowed to stir at about 25° C. for 1 h. This solution was then added to a solution of 1,3-dichloro-propan-2-one O-methyloxime (3.6 g, 20.6 mmol) in DMSO (13 ml) at 0° C. and stirred at about 25° C. for about 5 h. The reaction mixture was poured into ice water and extracted with EtAc (3×). The combined organic layers were dried (Na₂SO₄) and concentrated. Column purification (100-200 silica gel, 3-5% MeOH/DCM with 1-2 drops of TEA per 100 ml of eluent) provided 2.0 g of the product as light brown solid.

### 11c) 5-(Dimethylamino-methyleneamino)-1-[2-methoxyimino-3-(3-trifluoro-methyl-phenoxy)-propyl]-1H-indole-3-carboxylic acid amide

To a solution of 3-(trifluoromethyl)phenol (255 mg, 1.57 mmol) in DMSO (3.4 ml) was added KOtBu (177 mg, 1.5 mmol) at about 25° C. and stirred for 1 h. This solution was added to a solution of 1-{3-chloro-2-[methoxyimino]-propyl}-5-(dimethylaminomethylene-amino)-1H-indole-3-carboxylic acid amide (500 mg, 1.4 mmol) in DMSO (1.7 ml) and allowed to stir at about 25° C. for about 12 h. LC-MS revealed clean conversion. Reaction mixture was poured into ice water and extracted with EtAc (3×). The combined organic layers were dried (Na₂SO₄) and column purification (70-230 mesh basic alumina, 15% EtAc/hexane) provided 90 mg of the desired compound as solid.

### Example 12

### 5-(Dimethylamino-methyleneamino)-1-[2-methoxyimino-3-(4-methoxyphenoxy)-propyl]-1H-indole-3-carboxylic acid amide

A suspension of 1-{3-chloro-2-[methoxyimino]-propyl}-5-(dimethylamino-methyleneamino)-1H-indole-3-carboxylic acid amide (600 mg, 1.7 mmol, 1.0 eq), 4-methoxy aniline (233 mg, 1.89 mmol, 1.1 eq) and sodium carbonate (720 g, 6.8 mmol, 4.0 eq) in acetonitrite (8 ml) was refluxed for 24 h. LC-MS revealed completion of reaction. Ethyl acetate was added to the reaction mixture and organic layer was washed with water, brine and dried (sodium sulfate). Concentration and column purification (70-230 mesh basic alumina, 15% ethyl acetate/hexane) provided 100 mg of the product as an off white solid.

### Example 13

### N′-{1-[2-Methoxyimino-3-(3-trifluoromethyl-phenoxy)-propyl]-2-methyl-1H-indol-5-yl}-N,N-dimethyl-acetamidine

### 13a) N,N-Dimethyl-N′-(2-methyl-1H-indol-5-yl)-acetamidine

A mixture of 2-methyl-1H-indol-5-ylamine (3 g, 20.7 mmol) and N,N-dimethyl-acetamide dimethylacetal (3.6 ml, 25 mmol, 1.2 eq) was heated at 70° C. for 30 mins. Reaction mixture became solid and TLC revealed clean conversion. Residue was triturated with hexane (3 times) to obtain 4.1 g (92%) of the product as dark brown solid.

### 13b) N′-(1-{3-Chloro-2-[methoxyimino]-propyl}-2-methyl-1H-indol-5-yl)-N,N-dimethylacetamidine

To a solution of N,N-dimethyl-N′-(2-methyl-1H-indol-5-yl)-acetamidine (4 g, 18.5 mmol) in dry DMSO (44 ml) was added potassium tert.-butylate (KOtBu) (2.1 g, 18.5 mmol) and it was allowed to stir at room temperature for 1 h. This above solution was then added to a solution of 1,3-dichloro-propan-2-one-O-methyloxime (5.8 g, 37 mmol) in DMSO (20 ml) at 0° C. and stirred at room temperature for 5 h. Reaction mixture was poured into ice water and extracted 3 times with MTBE. The combined organic layer have been dried with sodium sulfate and concentrated. Column purification (100-200 silica gel, 3-5% MeOH/methylene chloride) provided 3.2 g (52%) of the title compound as dark brown sticky material.

### 13c) N′-{1-[2-Methoxyimino-3-(3-trifluoromethyl-phenoxy)-propyl]-2-methyl-1H-indol-5-yl}-N,N-dimethyl-acetamidine

To a solution of 3-trifluoromethyl phenol (400 mg, 2.5 mmol) in DMSO (5.4 ml) have been added KOtBu (277 mg, 2.47 mmol) at room temperature and stirred for 1 h. This solution was then added to a solution of N′-(1-{3-Chloro-2-[methoxyimino]-propyl}-2-methyl-1H-indol-5-yl)-N,N-dimethyl-acetamidine (750 mg, 2.24 mmol) in DMSO (2.5 ml) and allowed to stir at room temperature for about 12 h. TLC was inconclusive (as both the product and starting material have similar Rf values) but LC MS revealed clean conversion. Reaction mixture was poured into ice water and extracted with methyl-tert.-butylether (3 times). Combined organic layer was dried (Na₂SO₄) and column purification (70-230 mesh basic alumina, 10% ethyl acetate/hexane) provided 260 mg of the desired compound as a yellow sticky material.

### Example 14

### N′-{1-[2-Methoxyimino-3-(4-methoxy-phenoxy)-propyl]-2-methyl-1H-indol-5-yl}-N,N-dimethyl-acetamidine

A suspension of N′-(1-{3-chloro-2-[nnethoxyimino]-propyl}-2-methyl-1H-indol-5-yl)-N,N-dimethyl-acetamidine (750 mg, 2.2 mmol), 4-methoxy aniline (304 mg, 2.47 mmol) and Na₂CO₃ (950 mg, 8.9 mmol) in acetonitrile (6 ml) was refluxed for 24 h and cooled to room temperature. The reaction mixture was poured into water and extracted with MTBE (3 times). The combined organic layers have been dried (Na₂SO₄) and concentrated. Column purification (70-230 mesh basic alumina, 10% EtOAc/hexane) provided 235 mg of the desired compound as a brown sticky material.

### Example 15

### N′-[1-(3,4-Dichloro-benzyl)-1H-indol-5-yl]-N-ethyl-N-methyl-formamidine

### 15a) N′-(1H-indol-5-yl)-N-ethyl-N-methyl-formamidine

5-Aminoindole (4.0 g), trimethylorthoformiate (25.7 g), and catalytic amounts of p-toluenesulfonic acid monohydrate were heated together at 80° C. for 5 hours in a microwave oven, after which time the volatile components have been removed in vacuo and the residue dissolved in DCM (40 ml) and stirred with N-ethylmethylamine (3.6 g) at room temperature over night. After evaporation of the volatile components in vacuo, the residue was purified by column chromatography with a gradient of DCM and MeOH which yielded 2.2 g of the product.

### 15b) N′-[1-(3,4-Dichloro-benzyl)-1H-indol-5-yl]-N-ethyl-N-methyl-formamidine

Under argon, N′-(1H-indol-5-yl)-N-ethyl-N-methyl-formamidine (280 mg) was dissolved in dry DMSO (5 ml) at room temperature and potassium tert-butylate (156 mg) and 18-crown-6 (18 mg) were added and stirred for 5 minutes. Then 3,4-dichlorobenzylchloride (272 mg) dissolved in 5 ml of dry DMSO were added dropwise and the reaction mixture was stirred over night. Water (40 ml) was added and the mixture was extracted MTBE (3×40 ml). The combined organic phases were washed with water (2×30 ml) and dried over sodium sulfate. After evaporation of the solvents, the residue was purified by column chromatography with a gradient of DCM and MeOH which yielded 280 mg of the product.

### Example 16

### N′-[1-(2,4-Dichloro-benzyl)-2-methyl-1H-indol-5-yl]-N-ethyl-N-methylformamidine

### 16a) N′-(2-Methyl-1H-indol-5-yl)-N-ethyl-N-methyl-formamidine

2-Methyl-5-aminoindole (2.0 g), trimethylorthoformiate (11.6 g), and catalytic amounts of p-toluenesulfonic acid monohydrate (0.2 g) were heated together at 80° C. for 5 hours in a microwave oven, after which time the reaction mixture was reduced under vacuum and the residue dissolved in DCM (20 ml) and stirred with N-ethylmethylamine (1.6 g) at room temperature over night. After evaporation of the volatile components in vacuo, the residue was purified by column chromatography with a gradient of DCM and MeOH which yielded 1.4 g of the product.

### 16b) N′-[1-(2,4-Dichloro-benzyl)-2-methyl-1H-indol-5-yl]-N-ethyl-N-methyl-formamidine

Under argon, sodium hydride (66 mg) and 15-crown-5 (16 mg) were added to 3 ml of dry THF at 0° C. and N′-(2-Methyl-1H-indol-5-yl)-N-ethyl-N-methyl-formamidine (320 mg) dissolved in 3 ml of dry THF was added dropwise and stirred for 10 minutes. Then 2,4-dichlorobenzylchloride (291 mg) dissolved in 4 ml of dry THF were added dropwise and the reaction mixture was stirred over night. Water (40 ml) was added and the mixture was extracted with MTBE (3×40 ml). The combined organic layers were washed with water (2×30 ml) and dried over sodium sulfate. After evaporation of the solvents, the residue was purified by column chromatography with a gradient of DCM and MeOH which yielded 40 mg of the product.

### Example 17

### N′-(1-{3-(2-Chloro-6-fluoro-phenoxy)-2-[methoxyimino]-propyl}-2-methyl-1H-indol-5-yl)-N-ethyl-N-methyl-formamidine

Under argon, N′-(2-Methyl-1H-indol-5-yl)-N-ethyl-N-methyl-formamidine (700 mg) was dissolved in dry DMSO (5 ml) at room temperature and potassium tert-butylate (219 mg) and 18-crown-6 (26 mg) were added and stirred for 5 minutes. Then 1-chloro-3-(2-chloro-6-fluoro-phenoxy)-propan-2-one-O-methyl-oxime (611 mg) dissolved in 5 ml of dry DMSO were added dropwise and the reaction mixture was stirred over night. Water (40 ml) was added and the mixture was extracted with MTBE (3×40 ml). The combined organic phases were washed with water (2×30 ml) and dried over sodium sulfate. After evaporation of the solvents, the residue was purified by column chromatography with a gradient of DCM and MeOH which yielded 190 mg of the product.

### Example 18

### N′-[1-(2-Chloro-6-fluoro-benzyl)-2,3-dimethyl-1H-indol-5-yl]-N-ethyl-N-methyl-formamidine

### 18a) N′-(2,3-Dimethyl-1H-indol-5-yl)-N-ethyl-N-methyl-formamidine

2,3-Dimethyl-5-aminoindole (2.0 g), trimethylorthoformiate (10.6 g), and catalytic amounts of p-toluenesulfonic acid monohydrate (0.2 g) were heated together at 80° C. for 5 hours in a microwave oven, after which time the volatile components have been removed in vacuo and the residue dissolved in DCM (20 ml) and stirred with N-ethylmethylamine (1.5 g) at room temperature over night. After evaporation of the volatile components in vacuo, the residue was purified by column chromatography with a gradient of DCM and MeOH which yielded 1.3 g of the product.

### 18b) N′-[1-(2-Chloro-6-fluoro-benzyl)-2,3-dimethyl-1H-indol-5-yl]-N-ethyl-N-methylformamidine

Under argon, N′-(2,3-dimethyl-1H-indol-5-yl)-N-ethyl-N-methyl-formamidine (650 mg) was dissolved in dry DMSO (5 ml) at room temperature and potassium tert-butylate (318 mg) and 18-crown-6 (37 mg) were added and stirred for 5 minutes. Then 2-chloro-6-fluorobenzylchloride (597 mg) dissolved in 5 ml of dry DMSO were added dropwise and the reaction mixture was stirred over night. Water (40 ml) was added and the mixture was extracted with MTBE (3×40 ml). The combined organic phases were washed with water (2×30 ml) and dried over sodium sulfate. After evaporation of the solvents, the residue was purified by column chromatography with a gradient of DCM and MeOH which yielded 200 mg of the product.

### Example 19

### N′-(2-Benzyl-3,6-dimethyl-benzofuran-5-yl)-N-ethyl-N-methyl-formamidine

### 19a) 4-Phenyl-butan-2-one O-(3-methyl-4-nitro-phenyl)-oxime

To 1.56 g (61.9 mmol) sodium hydride (95%) in 20 ml DMF a solution of 9.26 g (56.7 mmol) 4-phenyl-butan-2-one oxime in 60 ml DMF was added dropwise with stirring at room temperature. Stirring was continued for 15 min. Then 114 mg (0.52 mmol) crown ether (15-crown-5) and finally a solution of 8.00 g (51.6 mmol) 5-fluoro-2-nitrotoluene in 20 ml DMF have been added. Stirring was continued over night at room temperature. The reaction mixture was poured into 700 ml water and 150 ml MTBE. After collection of the organic layer the water was extracted 3× with 50 ml MTBE. The combined organic layers were washed 4 times with 60 ml of water each, dried with sodium sulfate, and the solvents were removed in vacuo. The crude product was purified by chromatography on silica (eluent cyclohexane/MTBE). Yield 11.5 g. 1H-NMR (CDCl₃): δ=1.57 (s); 1.97 (s); 2.10 (s); 2.65 (m); 2.95 (m); 7.00-7.40 (m); 8.07 (m) [mixture of isomeric oximes].

### 19b) Ring closure yielding a mixture of: 6-Methyl-5-nitro-2-phenethyl-benzofuran, 4-methyl-5-nitro-2-phenethyl-benzofuran, 2-benzyl-3,6-dimethyl-5-nitro-benzofuran, 2-benzyl-3,4-dimethyl-5-nitro-benzofuran

To 2.00 g (6.70 mmol) 4-phenyl-butan-2-one O-(3-methyl-4-nitro-phenyl)-oxime and 983 mg (8.04 mmol) dimethylaminipyridine in 20 ml methylene chloride 3.66 g (13.41 mmol) trifluoroacetyl trifluoromethanesulfonate have been added dropwise with stirring at 0° C. After stirring for 1 h at 0° C. the mixture was heated for 30 min to 70° C. in a microwave stove. The reaction mixture was poured into 60 ml water and 60 ml methyl-tert.-butyl ether. After collection of the organic layer the water was extracted 2 times with 20 ml MTBE each. The combined organic layers were washed 2 times with 15 ml of water each, dried with sodium sulfate, and the solvents were removed in vacuo. The crude product was purified by chromatography on silica (eluent cyclohexane/MTBE). Yield 1.7 g of a mixture of isomeric benzofurans.

### 19c) 4-Methyl-2-phenethyl-benzofuran-5-ylamine, 6-methyl-2-phenethyl-benzofuran-5-ylamine, 2-benzyl-3,6-dimethyl-benzofuran-5-ylamine, 2-benzyl-3,4-dimethylbenzofuran-5-ylamine

To 166 mg Pd on charcoal (10%) has been added a solution of 4.4 g of the above mixture (19b) in 8 ml MeOH and 8 ml THF under a nitrogen atmosphere. Then 0.1 bar of hydrogen pressure have been applied and the mixture was stirred for 2 h at room temperature. After filtration of the catalyst and evaporation of the solvent in vacuo 4.2 g of crude product have been obtained. This was separated by chromatography on silica (eluent cyclohexane/MTBE). While 6-methyl-2-phenethyl-benzofuran-5-ylamine and 4-methyl-2-phenethyl-benzofuran-5-ylamine could not be separated and were further processed as a mixture, 2-benzyl-3,6-dimethyl-benzofuran-5-ylamine and 2-benzyl-3,4-dimethyl-benzofuran-5-ylamine could be obtained as pure compounds.

### 19d) N′-(2-Benzyl-3,6-dimethyl-benzofuran-5-yl)-N-ethyl-N-methyl-formamidine

To 78.0 mg (0.90 mmol) N-ethyl-N-methylformamide in 6 ml 1,4-dioxane 109.8 mg (0.72 mmol) phosphoroxychloride have been added with stirring at room temperature. After stirring for 5 min 150 mg (0.60 mmol) 2-benzyl-3,6-dimethyl-benzofuran-5-ylamine have been added. Stirring was continued over night at room temperature. The reaction mixture was poured into 6 ml water and 6 ml MTBE, and 2 ml triethylamine were added. After collection of the organic layer the water was extracted 2× with 2 ml MTBE. The combined organic layers were washed 2× with 2 ml of water each, dried with sodium sulfate, and the solvents were removed in vacuo. The crude product was purified by chromatography on silica (eluent cyclohexane/MTBE/1% triethylamine). Yield 167 mg.

### Example 20

### N′-(2-Benzyl-3,4-dimethyl-benzofuran-5-yl)-N-ethyl-N-methyl-forrinamidine

To 78.0 mg (0.90 mmol) N-ethyl-N-methylformamide in 6 ml 1,4-dioxane 109.8 mg (0.72 mmol) phosphoroxychloride have been added with stirring at room temperature. After stirring for 5 min 150 mg (0.60 mmol) 2-benzyl-3,4-dimethyl-benzofuran-5-ylannine have been added. Stirring was continued over night at room temperature. The reaction mixture was poured into 6 ml water and 6 ml MTBE, and 2 ml triethylamine were added. After collection of the organic layer the water was extracted 2× with 2 ml MTBE. The combined organic layers were washed 2 times with 2 ml of water each, dried with sodium sulfate, and the solvents were removed in vacuo. The crude product was purified by chromatography on silica (eluent cyclohexane/MTBE/1% triethylamine). Yield 141 mg.

### Example 21

### N-Ethyl-N-methyl-N′-(6-methyl-2-phenethyl-benzofuran-5-yl)-formamidine and N-ethyl-N-methyl-N′-(4-methyl-2-phenethyl-benzofuran-5-yl)-formamidine

To 52.0 mg (0.60 mmol) N-ethyl-N-methylformamide in 2 ml 1,4-dioxane 73.2 mg (0.48 mmol) phosphoroxychloride have been added with stirring at room temperature. After stirring for 5 min 100 mg (0.40 mmol) of the mixture of 6-methyl-2-phenethylbenzofuran-5-ylamine and 4-methyl-2-phenethyl-benzofuran-5-ylamine obtained in example 19c) have been added in 1 ml of 1,4-dioxane. Stirring was continued over night at room temperature. The reaction mixture was poured into 6 ml water and 6 ml MTBE, and 2 ml triethylamine were added. After collection of the organic layer the water was extracted 2× with 2 ml MTBE. The combined organic layers were washed 2× with 2 ml of water each, dried with sodium sulfate, and the solvents were removed in vacuo. The crude material was separated by chromatography on silica (eluent cyclohexane/MTBE/2% triethylamine). Both products have been obtained in good purity.

The compounds listed in Table I-A have been prepared in an analogous manner.

TABLE I-A
Compounds of formulae I.1A to I.15B

| ex. no | form. | R¹, R², R³ (if not hydrogen or absent)* | L¹; L²; L³ | R⁵ | R⁶ | R⁷ |
|---|---|---|---|---|---|---|
| I-1 | I.13A | R¹ = 2-methoxyimino-3-(3-trifluoro- | H; H; H | CH₃ | CH₃ | H |
| | | methyl-phenoxy)-propyl, R³ = carbamoyl | | | | |
| I-2 | I.13A | R¹ = 2-methoxyimino-3-(3-trifluorometh- | H; H; H | CH₃ | CH₃ | H |
| | | yl-phenoxy)-propyl | | | | |
| I-3 | I.13A | R¹ = 2-methoxyimino-3-(3-trifluorometh- | H; H; H | CH₃ | CH₃ | H |
| | | yl-phenylamino)-propyl, R³ = carbamoyl | | | | |
| I-4 | I.13A | R¹ = 2-methoxyimino-3-(3-bromo- | H; H; H | CH₃ | CH₃ | H |
| | | phenylamino)-propyl, R³ = carbamoyl | | | | |
| I-5 | I.13A | R¹ = 2-methoxyimino-3-(2-fluoro- | H; H; H | CH₃ | CH₃ | H |
| | | phenoxy)-propyl, R³ = carbamoyl | | | | |
| I-6 | I.13A | R¹ = 2-methoxyimino-3-(2-fluoro- | H; H; H | CH₃ | CH₃ | H |
| | | phenoxy)-propyl | | | | |
| I-7 | I.13A | R¹ = 2-methoxyimino-3-(3,4-dichloro- | H; H; H | CH₃ | CH₃ | H |
| | | phenoxy)-propyl | | | | |
| I-8 | I.13A | R¹ = 2-methoxyimino-3-(4-methoxy- | H; H; H | CH₃ | CH₃ | H |
| | | phenylamino)-propyl | | | | |
| I-9 | I.13A | R¹ = 2-methoxyimino-3-(3,4-dichloro- | H; H; H | CH₃ | CH₃ | H |
| | | phenoxy)-propyl, R³ = carbamoyl | | | | |
| I-10 | I.13A | R¹ = 2-methoxyimino-3-(3,4-dichloro- | H; H; H | CH₃ | CH₃ | CH₃ |
| | | phenoxy)-propyl, R² = CH₃ | | | | |
| I-11 | I.13A | R¹ = 2-methoxyimino-3-(2-fluoro- | H; H; H | CH₃ | CH₃ | CH₃ |
| | | phenoxy)-propyl, R² = CH₃ | | | | |
| I-12 | I.13A | R¹ = 2-methoxyimino-3-(3-trifluorometh- | H; H; H | CH₃ | CH₃ | CH₃ |
| | | yl-phenoxy)-propyl, R² = CH₃ | | | | |
| I-13 | I.13A | R¹ = 2-methoxyimino-3-(4-methoxy- | H; H; H | CH₃ | CH₃ | H |
| | | phenylamino)-propyl, R³ = carbamoyl | | | | |
| I-14 | I.13A | R¹ = 2-methoxyimino-3-(4-methoxy- | H; H; H | CH₃ | CH₃ | CH₃ |
| | | phenylamino)-propyl, R² = CH₃ | | | | |
| I-15 | I.13A | R¹ = 2-methoxyimino-3-(2-chloro- | H; H; H | CH₃ | CH₂CH₃ | H |
| | | 6-fluoro-phenoxy)-propyl | | | | |
| I-16 | I.13A | R¹ = 3,4-dichloro-benzyl | H; H; H | CH₃ | CH₂CH₃ | H |
| I-17 | I.13A | R¹ = 2-methoxyimino-3-(2-chloro- | H; H; H | CH₃ | CH₂CH₃ | H |
| | | 6-fluoro-phenoxy)-propyl, | | | | |
| | | R³ = methoxyimino-methyl | | | | |
| I-18 | I.13A | R¹ = 2-methoxyimino-3-(2-chloro-6- | H; CH₃; H | CH₃ | CH₂CH₃ | H |
| | | methyl-phenoxy)-propyl | | | | |
| I-19 | I.13A | R¹ = 2-methoxyimino-3-(2-chloro- | H; CH₃; H | CH₃ | CH₂CH₃ | H |
| | | 6-fluoro-phenoxy)-propyl | | | | |
| I-20 | I.13A | R¹ = 2-methoxyimino-3-(2-chloro-6- | H; H; H | CH₃ | CH₂CH₃ | H |
| | | fluoro-phenoxy)-propyl, R² = CH₃ | | | | |
| I-21 | I.13A | R¹ = 3-trifluoromethyl-benzyl, | H; H; H | CH₃ | CH₂CH₃ | H |
| | | R³ = methoxyimino-methyl | | | | |
| I-22 | I.13A | R¹ = 2,4-dichloro-benzyl, R² = CH₃ | H; H; H | CH₃ | CH₂CH₃ | H |
| I-23 | I.13A | R¹ = 3-trifluoromethyl-benzyl, R² = CH₃ | H; H; H | CH₃ | CH₂CH₃ | H |
| I-24 | I.13A | R¹ = 2-chloro-6-fluoro-benzyl, R² = CH₃ | H; H; H | CH₃ | CH₂CH₃ | H |
| I-25 | I.13A | R¹ = 2-chloro-6-fluoro-benzyl, R² = CH₃, | H; H; H | CH₃ | CH₂CH₃ | H |
| | | R³ = CH₃ | | | | |
| I-26 | I.13A | R¹ = 3-trifluoromethyl-benzyl, R² = CH₃, | H;H;H | CH₃ | CH₂CH₃ | H |
| | | R³ = CH₃ | | | | |
| I-27 | I.13A | R¹ = 4-fluoro-benzyl | H; H; H | CH₃ | CH₂CH₃ | H |
| I-28 | I.13A | R¹ = 2,4-dichloro-benzyl | H; H; H | CH₃ | CH₂CH₃ | H |
| I-29 | I.13A | R¹ = 2-chloro-6-fluoro-benzyl | H; H; H | CH₃ | CH₂CH₃ | H |
| I-30 | I.6A | R¹ = 2-methoxyimino-3-(3-trifluorometh- | H; H; H | CH₃ | CH₃ | H |
| | | yl-phenoxy)-propyl | | | | |
| I-31 | I.6A | R¹ = 2-methoxyimino-3-(3-fluoro- | H; H; H | CH₃ | CH₃ | H |
| | | phenoxy)-propyl | | | | |
| I-32 | I.6A | R¹ = 2-isopropoxyimino-3-(3-trifluoro- | H; H; H | CH₃ | CH₃ | H |
| | | methyl-phenoxy)-propyl | | | | |
| I-33 | I.6A | R¹ = 2-methoxyimino-3-(3-trifluorometh- | H; H; H | CH₃ | CH₂CH₃ | H |
| | | yl-phenoxy)-propyl | | | | |
| I-34 | I.6A | R¹ = 2-methoxyimino-3-(3-fluoro- | H; H; H | CH₃ | CH₂CH₃ | H |
| | | phenoxy)-propyl | | | | |
| I-35 | I.6A | R¹ = 2-methoxyimino-3-(3-trifluorometh- | H; H; CH₃ | CH₃ | CH₃ | H |
| | | yl-phenoxy)-propyl | | | | |
| I-36 | I.6A | R¹ = 2-methoxyimino-3-(3-fluoro- | H; H; CH₃ | CH₃ | CH₃ | H |
| | | phenoxy)-propyl | | | | |
| I-37 | I.6A | R¹ = 2-isopropoxyimino-3-(3-trifluoro- | H; H; CH₃ | CH₃ | CH₃ | H |
| | | methyl-phenoxy)-propyl | | | | |
| I-38 | I.6A | R¹ = 2-methoxyimino-3-(3-trifluorometh- | H; H; CH₃ | CH₃ | CH₂CH₃ | H |
| | | yl-phenoxy)-propyl | | | | |
| I-39 | I.6A | R¹ = 2-methoxyimino-3-(3-fluoro- | H; H; CH₃ | CH₃ | CH₂CH₃ | H |
| | | phenoxy)-propyl | | | | |
| I-40 | I.6A | R¹ = 2-(3-trifluoromethyl-phenyl)-ethyl, | H; H; H | CH₃ | CH₃ | H |
| | | R² = CH₃, R³ = CH₃ | | | | |
| I-41 | I.6A | R¹ = 4-trifluoromethyl-benzyl | H; H; H | CH₃ | CH₃ | H |
| I-42 | I.6A | R¹ = 4-fluoro-benzyl | H; H; H | CH₃ | CH₃ | H |
| I-43 | I.6A | R¹ = 3-trifluoromethyl-benzyl | H; H; H | CH₃ | CH₃ | H |
| I-44 | I.6A | R¹ = 2-methoxyimino-3-(3-trifluorometh- | H; H; Cl | CH₃ | CH₂CH₃ | H |
| | | yl-phenoxy)-propyl | | | | |
| I-45 | I.6A | R¹ = 2-(3-trifluoromethyl-phenyl)-ethyl | H; H; CH₃ | CH₃ | CH₂CH₃ | H |
| I-46 | I.6A | R¹ = 3-trifluoromethyl-benzyl | H; H; CH₃ | CH₃ | CH₂CH₃ | H |
| I-47 | I.6A | R¹ = 4-fluoro-benzyl | H; H; CH₃ | CH₃ | CH₂CH₃ | H |
| I-48 | I.6A | R¹ = 3-trifluoromethyl-benzyl | H; H; H | CH₃ | CH₂CH₃ | H |
| I-49 | I.6A | R¹ = 2-methoxyimino-3-(3-trifluorometh- | CH₃; H; H | CH₃ | CH₂CH₃ | H |
| | | yl-phenoxy)-propyl | | | | |
| I-50 | I.6A | R¹ = 3-trifluoromethyl-benzyl | H; H; Cl | CH₃ | CH₂CH₃ | H |
| I-51 | I.6A | R¹ = 3-trifluoromethyl-benzyl | CH₃; H; H | CH₃ | CH₂CH₃ | H |
| I-52 | I.6A | R¹ = 4-fluoro-benzyl | CH₃; H; H | CH₃ | CH₂CH₃ | H |
| I-53 | I.15A | R¹ = 3-trifluoromethyl-benzyl | H; H; H | CH₃ | CH₃ | H |
| I-54 | I.15A | R¹ = 3-trifluoromethyl-benzyl | H; H; H | CH₃ | CH₂CH₃ | H |
| I-55 | I.15A | R¹ = 3-trifluoromethyl-benzyl | H; H; CH₃ | CH₃ | CH₂CH₃ | H |
| I-56 | I.15A | R¹ = 3-trifluoromethyl-benzyl | CH₃; H; H | CH₃ | CH₂CH₃ | H |
| I-57 | I.15A | R¹ = 4-fluoro-benzyl | CH₃; H; H | CH₃ | CH₂CH₃ | H |
| I-58 | I.15A | R¹ = 2-methoxyimino-3-(3-trifluorometh- | CH₃; H; H | CH₃ | CH₂CH₃ | H |
| | | yl-phenoxy)-propyl | | | | |
| I-59 | I.5A | R¹ = 2-methoxyimino-3-(3-trifluorometh- | H; H; H | CH₃ | CH₂CH₃ | H |
| | | yl-phenoxy)-propyl | | | | |
| I-60 | I.6A | R¹ = 2-methoxyimino-3-(3-trifluorometh- | H; H; F | CH₃ | CH₂CH₃ | H |
| | | yl-phenoxy)-propyl | | | | |
| I-61 | I.5A | R¹ = 4-trifluoromethyl-benzyl | H; H; CH₃ | CH₃ | CH₃ | H |
| I-62 | I.5A | R¹ = 2-methoxyimino-3-(3-trifluorometh- | H; H; H | CH₃ | CH₃ | H |
| | | yl-phenoxy)-propyl, R² = CH₃ | | | | |
| I-63 | I.5A | R¹ = 2-methoxyimino-3-(3-trifluorometh- | H; H; H | CH₃ | CH₂CH₃ | H |
| | | yl-phenoxy)-propyl, R² = CF₃ | | | | |
| I-64 | I.5A | R¹ = 2-methoxyimino-3-(3-fluoro- | H; H; CH₃ | CH₃ | CH₂CH₃ | H |
| | | phenoxy)-propyl | | | | |
| I-65 | I.5A | R¹ = 2-methoxyimino-3-(3-trifluorometh- | H; H; CH₃ | CH₃ | CH₃ | H |
| | | yl-phenoxy)-propyl | | | | |
| I-66 | I.5A | R¹ = 2-methoxyimino-3-(3-trifluorometh- | H; H; CH₃ | CH₃ | CH₂CH₃ | H |
| | | yl-phenoxy)-propyl | | | | |
| I-67 | I.5B | R¹ = 2-methoxyimino-3-(3-trifluorometh- | H; H; H | CH₃ | CH₃ | H |
| | | yl-phenoxy)-propyl | | | | |
| I-68 | I.5B | R¹ = 2-methoxyimino-3-(3-trifluorometh- | H; H; H | CH₃ | CH₂CH₃ | H |
| | | yl-phenoxy)-propyl | | | | |
| I-69 | I.5B | R¹ = 2-methoxyimino-3-(3-trifluorometh- | H; H; H | CH₃ | CH₃ | H |
| | | yl-phenoxy)-propyl, R² = CF₃ | | | | |
| I-70 | I.5B | R¹ = 2-methoxyimino-3-(3-fluoro- | H; H; H | CH₃ | CH₃ | H |
| | | phenoxy)-propyl, R² = CF₃ | | | | |
| I-71 | I.5B | R¹ = 2-methoxyimino-3-(3-fluoro- | H; H; H | CH₃ | CH₃ | H |
| | | phenoxy)-propyl, R² = CH₃ | | | | |
| I-72 | I.5B | R¹ = 2-methoxyimino-3-(3-fluoro- | H; H; H | CH₃ | CH₂CH₃ | H |
| | | phenoxy)-propyl, R² = CH₃ | | | | |
| I-73 | I.5B | R¹ = 2-methoxyimino-3-(3-fluoro- | H; H; H | CH₃ | CH₂CH₃ | H |
| | | phenoxy)-propyl, R² = CH₃ | | | | |
| I-74 | I.5B | R¹ = 2-methoxyimino-3-(3-trifluorometh- | H; H; H | CH₃ | CH₂CH₃ | H |
| | | yl-phenoxy)-propyl, R² = CH₃ | | | | |
| I-75 | I.3A | R¹ = 2-methoxyimino-3-(3-trifluorometh- | H; H; H | CH₃ | CH₂CH₃ | H |
| | | yl-phenoxy)-propyl, R² = CH₃ | | | | |
| I-76 | I.3A | R¹ = 2-methoxyimino-3-(3-fluoro- | H; H; H | CH₃ | CH₂CH₃ | H |
| | | phenoxy)-propyl, R² = CH₃ | | | | |
| I-77 | I.3A | R¹ = 3-trifluoromethyl-benzyl, R² = CH₃ | H; H; H | CH₃ | CH₂CH₃ | H |
| I-78 | I.3A | R¹ = 2-methoxyimino-3-(3-trifluorometh- | H; H; H | CH₃ | CH₂CH₃ | H |
| | | yl-phenoxy)-propyl | | | | |
| I-79 | I.3A | R¹ = 2-methoxyimino-3-(3-trifluorometh- | H; H; H | CH₃ | CH₃ | H |
| | | yl-phenoxy)-propyl | | | | |
| I-80 | I.3A1 | R¹ = 2-methoxyimino-3-(3-fluoro- | H; H; H | CH₃ | CH₃ | H |
| | | phenoxy)-propyl | | | | |
| I-81 | I.3A1 | R¹ = 2-methoxyimino-3-(3- | H; H; H | CH₃ | CH₃ | H |
| | | trifluoromethyl-phenoxy)-propyl | | | | |
| I-82 | I.3A1 | R¹ = 2-methoxyimino-3-(3-trifluorometh- | H; H; H | CH₃ | CH₂CH₃ | H |
| | | yl-phenoxy)-propyl | | | | |
| I-83 | I.7A | R¹ = benzyl, R³ = CH₃ | H; H; CH₃ | CH₃ | CH₂CH₃ | H |
| I-84 | I.7A | R¹ = 2-phenyl-ethyl | H; H; CH₃ | CH₃ | CH₂CH₃ | H |
| I-85 | I.7A | R¹ = benzyl, R³ = CH₃ | CH₃; H; H | CH₃ | CH₂CH₃ | H |
| I-86 | I.7A | R¹ = 2-phenyl-ethyl | CH₃; H; H | CH₃ | CH₂CH₃ | H |
| I-87 | I.4A | R¹ = 2-methoxyimino-3-(3-trifluorometh- | H; H; CH₃ | CH₃ | CH₂CH₃ | H |
| | | yl-phenoxy)-propyl | | | | |
| I-88 | I.4A | R¹ = 4-trifluoromethyl-benzyl | H; H; CH₃ | CH₃ | CH₂CH₃ | H |
| I-89 | I.4A | R¹ = 4-trifluoromethyl-benzyl | H; H; CH₃ | CH₃ | CH₃ | H |
| I-90 | I.4A | R¹ = 4-fluoro-benzyl | H; H; CH₃ | CH₃ | CH₂CH₃ | H |
| I-91 | I.6A | R¹ = 2-trifluoromethyl-benzyl | H; H; CH₃ | CH₃ | CH₂CH₃ | H |
| I-92 | I.6A | R¹ = 4-trifluoromethyl-benzyl | H; H; CH₃ | CH₃ | CH₂CH₃ | H |

For formula (form.), the number of the respective formula selected from I.1 to I.15B is given.
If in Column “R¹, R², R³” no substituent is designated for a substituent R¹, R² and/or R³ in a specific line, this shall mean that the respective substituent is hydrogen.
In Column “L¹; L²; L³” the substituents L¹, L² and L³ are designated and separated by a semicolon (“;”).

TABLE I-B
Physico-chemical data for compounds of formulae I.1A to I.15B as defined in Table I-A.

| | HPLC 1 | HPLC 2 | |
|---|---|---|---|
| ex. no | Rₜ (min) | Rₜ (min) | 1H-NMR (ppm) |
| I-1 | 3.575 | | (DMSO): δ = 2.97 (s. broad); 3.98 (s); 4.36 (s); 5.18 (s); |
| | | | 6.80 (d); 7.04-7.15 (m); 7.17-7.28 (m); 7.40-7.47 (m); |
| | | | 7.57 (s); 7.63 (s); 7.93 (s); |
| I-2 | 4.469 | | (DMSO): δ = 2.93 (s. broad); 3.98 (s); 4.23 (s); 5.17 (s); |
| | | | 6.25 (s); 6.76 (d); 6.96; 7.07 (s); 7.15 (d); 7.24 (s); 7.26 |
| | | | (s); 7.43 (t); 7.63 (s); |
| I-3 | 3.586 | | (DMSO): δ = 2.96 (s. broad); 3.55 (d); 3.92 (s); 5.04 (s); |
| | | | 6.37 (t); 6.71-6.89 (m); 7.19-7.28 (m); 7.40-7.47 (m); 7.59 |
| | | | (s); 7.67 (s); 7.91 (s); |
| I-4 | 4.432 | | (DMSO): δ = 2.96 (s. broad); 3.50 (d); 3.92 (s); 5.03 (s); |
| | | | 6.16 (t); 6.48 (d); 6.69 (d); 6.71 (s); 6.83 (d); 6.98 (t); 7.22 |
| | | | (d); 7.59 (s); 7.67 (s); 7.90 (s); |
| I-5 | 5.203 | | (DMSO): δ = 2.95 (s. broad); 3.97 (s); 4.30 (s); 5.17 (s); |
| | | | 6.80 (d); 6.90-7.05 (m); 7.14-7.27 (m); 7.58 (s); 7.65 (s); 7.89 (s); |
| I-6 | 3.854 | | (DMSO): δ = 2.93 (s. broad); 3.97 (s); 4.17 (s); 5.16 (s); |
| | | | 6.28 (d); 6.765 (d); 6.87-7.26 (m); 7.65 (s); |
| I-7 | 3.624 | | (DMSO): δ = 2.93 (s. broad); 3.98 (s); 4.16 (s); 5.16 (s); |
| | | | 6.25 (d); 6.76 (dd); 6.96 (s); 6.99 (d); 7.14 (d); 7.22 (d); |
| | | | 7.42 (d); 7.64 (s); |
| I-8 | 3.445 | | (DMSO): δ = 2.94 (s. broad); 3.62 (s); 3.92 (s); (NH)CH2 |
| | | | not detected; 5.02 (s); 6.32 (d); 6.45 (d); 6.67 (d); 6.79 |
| | | | (d); 7.01 (s); 7.18 (d); 7.26 (d); 7.67 (s); |
| I-9 | 5.540 | | (DMSO) both isomers: δ = 2.95 (s. broad); 3.90 (s); 3.98 |
| | main | | (s); 4.27 (s); 4.79 (s); 5.01 (s); 5.17 (s); 5.76 (s); 6.74- |
| | isomer | | 6.83 (m); 6.92 (dd); 6.05 (d); 7.19 (s); 7.20 (d); 7.33 (d); |
| | | | 7.42 (d); 7.50 (d); 7.57 (dd); 7.63 (s); 7.65 (s); 7.91 (s); 7.94 (s) |
| I-10 | 4.999 | | (DMSO): δ = 1.72 (s); 2.30 (s); 2.93 (s. broad); 3.99 (s); |
| | | | 4.04 (s); 5.12 (s); 6.03 (s); 6.30 (d); 6.52 (s); 6.71 (dd); |
| | | | 6.96 (s); 7.07 (d); 7.39 (d); |
| I-11 | 4.033 | | (DMSO): δ = 1.74 (s); 2.32 (s); 2.93 (s. broad); 3.99 (s); |
| | | | 4.04 (s); 5.12 (s); 6.04 (s); 6.31 (d); 6.55 (s); 6.78-7.29 (m); |
| I-12 | 4.615 | | (DMSO): δ = 1.71 (s); 2.30 (s); 2.93 (s. broad); 3.99 (s); |
| | | | 4.13 (s); 5.13 (s); 6.02 (s); 6.31 (d); 6.53 (s); 6.99 (d); |
| | | | 7.02 (s); 7.08 (d); 7.22 (d); 7.40 (t); |
| I-13 | 4.348 | | (DMSO) both isomers: δ = 2.96 (s. broad); 3.44 (d); 3.62 |
| | | | (s); 3.66 (s); 3.84 (s); 3.92 (s); 4.77 (s); 5.02 (s); 5.41 (t); |
| | | | 5.67 (t); 6.50 (m[c]); 6.68 (d); 6.76 (d); 6.78 (d); 6.83 (d); |
| | | | 7.16 (d); 7.22 (d); 7.58 (d); 7.66 (s); 7.85 (s); 7.90 (s) |
| I-14 | 3.690 | | (DMSO): δ = 1.80 (s); 2.30 (s); 2.95 (s. broad); 3.18 (d); |
| | | | 3.62 (s); 3.93 (s); 5.03 (s); 5.76 (s); 6.09 (d); 6.30-6.42 |
| | | | (m); 6.59-6.67 (m); 7.06 (d); |
| I-15 | | 2.997 | (CDCl₃): δ = 1.21 (t); 3.02 (s); 3.38 (broad); 3.98 (s); 4.27 |
| | | | (s); 5.22 (s); 6.42 (s); 6.88-7.04 (m); 7.11-7.25 (m); 7.59 |
| | | | (broad) (main isomer) |
| I-16 | | 2.886 | (CDCl₃): δ = 1.22 (t); 3.03 (s); 3.39 (broad); 5.22 (s); 6.48 |
| | | | (d); 6.89 (m[c]); 7.05 (d); 7.09 (d); 7.18 (m); 7.33 (d); 7.59 (broad); |
| I-17 | | 3.045 | (CDCl₃): δ = 1.24 (t); 3.05 (s); 3.39 (broad); 3.98 (s); 3.99 |
| | | | (s); 4.28 (s); 5.20 (s); 6.94-7.12 (m); 7.11-7.29 (m); 7.33- |
| | | | 7.47(m); 7.58-7.70 (m); 8.14-8.24 (m) (main isomer) |
| I-18 | | | (CDCl₃): δ = 1.18-1.38 (m); 2.08 (s); 2.09 (s); 2.63 (s); |
| | | | 2.75 (s); 3.12-3.24 (m); 3.41-3.71 (m); 3.80 (s); 4.00 (s); |
| | | | 4.12 (s); 4.70 (s); 5.40 (s); 5.45 (s); 6.50 (d); 6.88 (s); |
| | | | 6.87-7.09 (m); 7.10-7.25 (m); 7.37 (s); 7.91 (broad) (both isomers) |
| I-19 | | | (CDCl₃): δ = 1.29 (t); 2.61 (s); 3.17 (s); 3.49 (broad); 3.95 |
| | | | (s); 4.21 (s); 5.46 (s); 6.47 (d); 6.88 (s); 6.90-7.10 (m); |
| | | | 7.11-7.26 (m); 7.80 (broad); |
| I-20 | | 3.142 | (CDCl₃): δ = 1.19 (t); 2.45 (s); 3.02 (s); 3.37 (broad); 3.96 |
| | | main | (s); 4.74 (s); 5.17 (s); 6.21 (s); 6.82-7.01 (m); 7.04-7.21 |
| | | isomer | (m); 7.35 (d); 7.69 (broad) (main isomer) |
| I-21 | | 3.079 | (CDCl₃): δ = 1.21 (t); 3.00 (s); 3.35 (broad); 3.98 (s); 5.27 |
| | | main | (s); 6.85-7.76 (m); 8.10 (s); 8.24 (s) (main isomer) |
| | | isomer | |
| I-22 | | 3.334 | (CDCl₃): δ = 1.22 (t); 2.29 (s); 3.13 (s); 3.42 (broad); 5.23 |
| | | | (s); 6.08 (d); 6.32 (s); 6.90-7.10 (m); 7.41 (s); 7.46 (s); 7.91 (broad); |
| I-23 | | 3.127 | (CDCl₃): δ = 1.21 (t); 2.32 (s); 3.03 (s); 3.38 (broad); 5.29 |
| | | | (s); 6.26 (s); 6.85 (dd); 6.98 (d); 7.02 (d); 7.14 (s); 7.35 |
| | | | (s); 7.41-7.55 (m); 7.61 (broad); |
| I-24 | | 2.982 | (CDCl₃): δ = 1.18 (t); 2.41 (s); 2.99 (s); 3.32 (broad); 5.35 |
| | | | (s); 6.17 (s); 6.84 (dd); 6.94 (m[c]); 7.05-7.26 (m); 7.62 (broad); |
| I-25 | | 3.200 | (CDCl₃): δ = 1.13 (t); 2.18 (s); 2.28 (s); 2.96 (s); 3.30 |
| | | | (broad); 5.28 (s); 6.78-6.94 (m); 7.05-7.21 (m); 7.69 (broad); |
| I-26 | | 3.235 | (CDCl₃): δ = 1.19 (t); 2.23 (s); 2.24 (s); 3.01 (s); 3.37 |
| | | | (broad); 5.24 (s); 6.88 (dd); 6.96 (d); 7.01 (d); 7.20 (s); |
| | | | 7.31 (s); 7.45 (d); 7.73 (broad); |
| I-27 | | 2.656 | (CDCl₃): δ = 1.18 (t); 2.98 (s); 3.32 (broad); 5.15 (s); 6.42 |
| | | | (s); 6.81-7.21 (m); 7.56 (broad); |
| I-28 | | 3.118 | (CDCl₃): δ = 1.19 (t); 2.98 (s); 3.32 (broad); 5.28 (s); |
| | | | 6.39-6.49 (m); 6.88 (dd); 6.95-7.09 (m); 7.18 (s); 7.39 (s); 7.58 (broad); |
| I-29 | | 2.843 | (CDCl₃): δ = 1.19 (t); 2.99 (s); 3.31 (broad); 5.38 (s); |
| | | | 6.33-6.47 (m); 6.77 (m[c]); 6.88 (dd); 6.90-7.05 (m); 7.05- |
| | | | 7.25 (m); 7.30 (s); 7.42 (d); 7.59 (broad); |
| I-30 | | | (CDCl₃): δ = 3.03 (s); 4.00 (s); 4.03 (s); 4.36 (s); 4.74 (s); |
| | | | 5.17 (s); 5.39 (s); 6.79-7.03 (m); 7.09-7.40 (m); 7.49 (s); |
| | | | 7.54 (s); 7.85 (s); 7.89 (s) |
| I-31 | | | (CDCl₃): δ = 3.04 (s); 3.98 (s); 4.02 (s); 4.29 (s); 4.72 (s); |
| | | | 5.13 (s); 5.37 (s); 6.35 (m); 6.43-6.68 (m); 7.05-7.19 (m); |
| | | | 7.29-7.38 (m); 7.51 (s); 7.56 (s); 7.85 (s); 7.91 (s) |
| I-32 | | | (CDCl₃): δ = 1.28 (m); 3.03 (s); 4.40 (s); 4.47 (m); 4.74 |
| | | | (s); 5.18 (s); 5.37 (s); 6.82 (m); 6.91 (s); 6.96-7.39 (m); |
| | | | 7.51 (s); 7.54 (s); 7.86 (s); 7.90 (s) |
| I-33 | | | (CDCl₃): δ = 1.22 (t); 3.01 (s); 3.35 (bs); 4.01 (s); 4.05 (s); |
| | | | 4.35 (s); 4.73 (s); 5.17 (s); 5.39 (s); 6.79-7.04 (m); 7.09- |
| | | | 7.41 (m); 7.52 (bs); 7.84 (s); 7.89 (s) |
| I-34 | | | (CDCl₃): δ = 1.23 (m); 3.04 (s); 3.38 (bs); 3.99 (s); 4.05 |
| | | | (s); 4.30 (s); 4.41 (s); 4.71 (s); 5.15 (s); 5.30 (s); 5.39 (s); |
| | | | 6.35 (m); 6.46 (m); 6.53-6.69 (m); 7.06-7.20 (m); 7.30 |
| | | | (m); 7.57 (bs); 7.87 (s) |
| I-35 | | | (CDCl₃): δ = 2.32-2.41 (m); 3.06 (bs); 3.91-4.08 (m); 4.31 |
| | | | (s); 4.43 (s); 4.72 (s); 5.11 (s); 5.36 (s); 6.75-7.45 (m); |
| | | | 7.76 (s); 7.79 (s); 7.82 (s) |
| I-36 | | | (CDCl₃): δ = 2.37 (m); 3.05 (s); 3.91-4.08 (m); 4.26 (s); |
| | | | 4.38 (m); 4.70 (s); 4.91 (s); 5.11 (s); 5.37 (s); 6.31-6.77 |
| | | | (m); 6.92 (s); 7.03-7.31 (m); 7.41 (m); 7.80 (s) |
| I-37 | | | (CDCl₃): δ = 1.31 (m); 2.37 (s); 3.04 (s); 4.38 (s); 4.48 |
| | | | (m); 5.30 (s); 5.36 (s); 6.82-6.95 (m); 7.10-7.31 (m); 7.39 |
| | | | (m); 7.80 (s) |
| I-38 | | | (CDCl₃): δ = 1.21 (m); 2.35 (s); 2.37 (s); 2.99 (s); 3.37 |
| | | | (bs); 3.98 (s); 4.03 (s); 4.30 (s); 4.43 (s); 4.72 (s); 5.12 |
| | | | (s); 5.38 (s); 6.73-6.99 (m); 7.02-7.37 (m); 7.41 (m); 7.79 |
| | | | (s); 7.83 (s) |
| I-39 | | | (CDCl₃): δ = 1.22 (t); 2.34 (s); 3.04 (s); 3.37 (bs); 3.97 (s); |
| | | | 4.01 (s); 4.24 (s); 4.35 (s); 4.67 (s); 5.10 (s); 5.29 (s); |
| | | | 5.37 (s); 6.32 (m); 6.41-6.68 (m); 6.89 (s); 7.04-7.22 (m); |
| | | | 7.40 (bs); 7.79 (s) |
| I-40 | | | (CDCl₃): δ = 1.68 (bs); 3.05 (s); 3.28 (t); 4.57 (t); 7.05 (s); |
| | | | 7.16 (s); 7.21 (m); 7.28-7.32 (m); 7.41 (m); 7.54 (m); 7.90 (s) |
| I-41 | | | (CDCl₃): δ = 3.11 (s); 5.61 (s); 7.18-7.37 (m); 7.53 (m); |
| | | | 7.63 (s); 7.97 (s) |
| I-42 | | | (CDCl₃): δ = 3.04 (s); 5.52 (s); 6.91-6.99 (m); 7.08 (m); |
| | | | 7.11-7.24 (m); 7.52 (m); 7.90 (s) |
| I-43 | | | (CDCl₃): δ = 1.74 (bs); 3.03 (s); 5.60 (s); 7.11 (m); 7.18- |
| | | | 7.25 (m); 7.30 (m); 7.40 (m); 7.46-7.58 (m); 7.95 (s) |
| I-44 | | | (CDCl₃): δ = 1.25 (t); 3.06 (s); 3.31 (bs); 3.56 (bs); 4.03 |
| | | | (s); 4.34 (s); 5.34 (s); 6.85 (m); 6.91 (m); 7.03 (bs); 7.14 |
| | | | (m); 7.28 (m); 7.47 (bs); 7.80 (s) |
| I-45 | | | (CDCl₃): δ = 1.21 (m); 2.34 (s); 3.00 (s); 3.23 (t); 3.36 |
| | | | (bs); 4.52 (t); 6.92 (m); 7.20-7.48 (m); 7.82 (s) |
| I-46 | | | (CDCl₃): δ = 1.21 (t); 2.39 (s); 3.02 (s); 3.37 (bs); 5.55 (s); |
| | | | 7.03 (s); 7.09 (s); 7.27 (m); 7.39 (m); 7.41-7.54 (m); 7.79 (s) |
| I-47 | | | (CDCl₃): δ = 1.21 (t); 2.35 (s); 3.01 (s); 3.36 (bs); 5.49 (s); |
| | | | 6.92-7.03 (m); 7.09-7.18 (m); 7.46 (s); 7.88 (s) |
| I-48 | | | (CDCl₃): δ = 1.22 (t); 1.80 (bs); 3.01 (s); 3.37 (bs); 5.60 |
| | | | (s); 7.08 (m). 7.19 (m); 7.28 (m); 7.36 (m); 7.45-7.59 (m); 7.95 (s) |
| I-49 | | | (CDCl₃): δ = 1.22 (t); 2.45 (s); 3.04 (s); 3.39 (bs); 4.03 (s); |
| | | | 4.32 (s); 4.71 (s); 5.13 (s); 5.38 (s); 6.81 (m); 6.87 (s); |
| | | | 6.92 (m); 7.11 (m); 7.20-7.31 (m); 7.40 (bs); 7.91 (s) |
| I-50 | | | (CDCl₃): δ = 1.22 (t); 3.07 (bs); 3.32-3.59 (m); 5.59 (s); |
| | | | 7.17 (bs). 7.27 (m); 7.35-7.49 (m); 7.53 (m); 7.91 (s) |
| I-51 | | | (CDCl₃): δ = 1.23 (t); 2.52 (s); 3.08 (bs); 3.40 (bs); 5.58 |
| | | | (s); 6.94 (m); 7.05 (m); 7.28 (m); 7.35-7.53 (m); 8.01 (s) |
| I-52 | | | (CDCl₃): δ = 1.20 (t); 2.51 (s); 3.01 (s); 3.36 (bs); 5.52 (s); |
| | | | 6.85-6.97 (m); 7.04 (m); 7.13 (m); 7.42 (bs); 7.98 (s) |
| I-53 | | | (CDCl₃): δ = 3.03 (s); 5.59 (s); 7.01 (s); 7.09 (m); 7.38- |
| | | | 7.50 (m); 7.53-7.65 (m); 7.79 (s) |
| I-54 | | | (CDCl₃): δ = 1.23 (t); 3.08 (bs); 3.39 (bs); 5.62 (s); 7.12 |
| | | | (m); 7.38-7.49 (m); 7.52-7.69 (m); 7.82 (s) |
| I-55 | | | (CDCl₃): δ = 1.22 (t); 2.37 (s); 3.02 (bs); 3.36 (bs); 5.58 |
| | | | (s); 6.82 (s); 7.29-7.60 (m); 7.72 (s) |
| I-56 | | | (CDCl₃): δ = 1.23 (t); 2.44 (s); 3.10 (bs); 3.42 (bs); 5.63 |
| | | | (s); 6.97 (m); 7.39-7.62 (m); 7.84 (s) |
| I-57 | | | (CDCl₃): δ = 1.25 (t); 2.41 (s); 3.08 (bs); 3.41 (bs); 5.53 |
| | | | (s); 6.94 (m); 7.05 (m); 7.24 (m); 7.49 (m); 7.78 (s) |
| I-58 | | | (CDCl₃): δ = 1.21 (t); 2.48 (s); 3.01 (bs); 3.36 (bs); 4.00 |
| | | | (s); 4.72 (s); 5.13 (s); 6.93 (m); 7.02 (s); 7.15-7.22 (m); |
| | | | 7.31 (m); 7.42 (bs); 7.92 (s) |
| I-59 | | | (CDCl₃): δ = 1.22 (m); 1.84 (bs); 3.02 (s); 3.37 (bs); 4.00 |
| | | | (s); 4.08 (s); 4.34 (s); 4.77 (s); 4.98 (s); 5.13 (s); 6.91- |
| | | | 7.10 (m); 7.19-7.43 (m); 7.58 (bs); 7.85 (s); 7.91 (s) |
| I-60 | | | (CDCl₃): δ = 1.25 (t); 3.08 (bs); 3.34 (bs); 3.57 (bs); 4.04 |
| | | | (s); 4.38 (s); 5.33 (s); 6.81-6.91 (m); 7.04-7.18 (m); 7.27 |
| | | | (m); 7.55 (bs); 7.83 (s) |
| I-61 | | | (CDCl₃): δ = 2.37 (s); 3.08 (s); 5.38 (s); 6.97 (s); 7.18 (s); |
| | | | 7.23 (m); 7.49 (s); 7.59 (m); 7.81 (s) |
| I-62 | | | (CDCl₃): δ = 2.55 (s); 3.00 (s); 4.08 (s); 4.25 (s); 5.09 (s); |
| | | | 6.85-7.03 (m); 7.08-7.40 (m); 7.49-7.56 (m) |
| I-63 | | | (CDCl₃): δ = 1.22 (m); 3.03 (s); 3.33 (bs); 3.89 (s); 4.08 |
| | | | (s); 4.32 (s); 4.83 (s); 5.17 (s); 5.36 (s); 6.75 (m); 6.82 (s); |
| | | | 6.96-7.16 (m); 7.21-7.44 (m); 7.49-7.60 (m) |
| I-64 | | | (CDCl₃): δ = 1.21 (m); 2.34 (s); 2.38 (s); 3.01 (s); 3.36 |
| | | | (bs); 3.98 (s); 4.07 (s); 4.26 (s); 4.70 (s); 4.92 (s); 5.09 |
| | | | (s); 6.51-6.74 (m); 7.07-7.29 (m); 7.45 (bs); 7.75 (s); 7.80 (s) |
| I-65 | | | (CDCl₃): δ = 2.38 (s); 2.41 (s); 3.11 (bs); 4.00 (s); 4.07 |
| | | | (s); 4.35 (s); 4.76 (s); 4.98 (s); 5.12 (s); 6.91-7.11 (m); |
| | | | 7.13-7.31 (m); 7.37 (m); 7.45 (s); 7.82 (s); 7.89 (s) |
| I-66 | | | (CDCl₃): δ = 1.22 (t); 1.73 (bs); 2.25 (s). 2.28 (s); 2.30 (s); |
| | | | 2.40 (s); 3.02 (s); 3.38 (bs); 4.03 (s); 4.09 (s); 4.37 (s); |
| | | | 4.75 (s); 4.91 (s); 4.95 (s); 5.10 (s); 5.13 (s); 6.92-7.15 |
| | | | (m); 7.18-7.27 (m); 7.30-7.47 (m); 7.51 (bs); 7.73-7.84 (m) |
| I-67 | | | (CDCl₃): δ = 3.04 (m); 3.99 (s); 4.06 (s); 4.36 (s); 4.77 (s); |
| | | | 4.95 (s); 5.10 (s); 6.93 (m); 7.01-7.11 (m); 7.18-7.40 (m); |
| | | | 7.49 (s); 7.53 (s); 7.62 (m); 7.70 (s); 7.76 (s) |
| I-68 | | | (CDCl₃): δ = 1.21 (t); 1.72 (bs); 3.03 (m); 3.34 (bs); 4.00 |
| | | | (s); 4.08 (s); 4.36 (s); 4.77 (s); 4.96 (s); 5.11 (s); 6.93 (m); |
| | | | 7.03 (m); 7.09 (m); 7.22 (m); 7.34 (m); 7.55 (m); 7.61 (s); |
| | | | 7.65 (s); 7.81 (s); 7.88 (s) |
| I-69 | | | (CDCl₃): δ = 3.02 (s); 3.85 (s); 4.07 (s); 4.33 (s); 4.88 (s); |
| | | | 5.16 (s); 5.31 (s); 6.70 (m); 6.81 (s); 6.89 (s); 6.95-7.03 |
| | | | (m); 7.10 (m); 7.21 (m); 7.34 (m); 7.44 (s); 7.52 (s); 7.62-7.71 (m) |
| I-70 | | | (CDCl₃): δ = 3.06 (s); 3.82 (s); 4.05 (s); 4.28 (m); 4.81 (s); |
| | | | 5.13 (s); 6.34 (m); 6.54-6.73 (m); 6.88-7.14 (m); 7.20- |
| | | | 7.32 (m); 7.44-7.53 (m); 7.65-7.72 (m) |
| I-71 | | | (CDCl₃): δ = 2.58 (s); 3.02 (s); 4.07 (s); 4.20 (s); 5.09 (s); |
| | | | 6.45-6.52 (m); 6.61 (m); 6.80 (s); 6.85 (m); 7.13 (m); 7.50 (m) |
| I-72 | | | (CDCl₃): δ = 1.21 (m); 2.09 (bs); 2.54 (s); 2.60 (s); 3.01 |
| | | | (s); 3.32 (bs); 3.94 (s); 4.08 (s); 4.19 (s); 4.70 (s); 4.89 |
| | | | (s); 5.10 (s); 6.45-6.52 (m); 6.58-6.73 (m); 6.78-6.90 (m); |
| | | | 6.97 (m); 7.10-7.25 (m); 7.58-7.68 (m) |
| I-73 | | | (CDCl₃): δ = 1.21 (m); 1.60 (bs); 3.04 (s); 3.33 (bs); 3.83 |
| | | | (s); 4.08 (s); 4.29 (s); 4.82 (s); 5.15 (s); 6.25-6.42 (m); |
| | | | 6.56-6.75 (m); 6.89-7.11 (m); 7.20-7.30 (m); 7.57 (bs); |
| | | | 7.70 (s); 7.75 (s) |
| I-74 | | | (CDCl₃): δ = 1.20 (m); 2.54 (s); 2.58 (s); 2.91 (bs); 3.01 |
| | | | (s); 3.31 (bs); 3.95 (s); 4.05 (s); 4.25 (s); 4.74 (s); 4.87 |
| | | | (s); 5.07 (s); 6.80 (s); 6.87 (m); 6.99 (m); 7.10-7.39 (m); |
| | | | 7.48-7.58 (m) |
| I-75 | | | (CDCl₃): δ = 1.18 (t); 1.27 (t); 2.52 (m); 2.94 (m); 3.03 |
| | | | (m); 3.30 (bs); 3.58-3.75 (m); 3.81-4.00 (m); 4.13 (m); |
| | | | 4.56-4.68 (m); 4.79-4.91 (m); 6.05 (s); 6.22-6.30 (m); |
| | | | 6.87 (m); 7.08 (m); 7.13-7.24 (m); 7.35 (m); 7.49 (m) |
| I-76 | | | (CDCl₃): δ = 1.18 (t); 1.27 (t); 2.51 (m); 2.91-3.10 (m); |
| | | | 3.30 (bs); 3.55-3.75 (m); 3.81-4.02 (m); 4.12 (m); 4.49- |
| | | | 4.60 (m); 4.74-4.88 (m); 6.03 (s); 6.21-6.30 (m); 6.58- |
| | | | 6.72 (m); 6.83-6.95 (m); 7.11-7.24 (m); 7.47 (m) |
| I-77 | | | (CDCl₃): δ = 1.17 (t); 1.28 (d); 1.71 (bs); 2.66 (m); 2.95 |
| | | | (s); 3.14 (m); 3.30 (bs); 3.71 (m); 4.29 (s); 5.98 (s); 6.27 |
| | | | (d); 6.95 (d); 7.38-7.65 (m) |
| I-78 | | | (CDCl₃): δ = 1.20 (t); 1.67 (bs); 2.89 (m); 3.05 (s); 3.34 |
| | | | (m); 3.82 (s); 3.90 (m); 4.03 (s); 4.67 (s); 4.93 (s); 6.35 |
| | | | (m); 6.52 (m); 6.73 (s); 6.82 (s); 7.10 (m); 7.15-7.25 (m); |
| | | | 7.38 (m); 7.48 (m) |
| I-79 | | | (CDCl₃): δ = 2.90 (m); 3.01 (s); 3.37 (m); 3.82 (s); 3.91 |
| | | | (m); 4.02 (s); 4.69 (s); 4.91 (s); 6.36 (m); 6.52 (m); 6.69 |
| | | | (m); 6.78 (s); 7.10 (m); 7.17-7.23 (m); 7.37 (m); 7.45 (m) |
| I-80 | | | (CDCl₃): δ = 3.03 (bs); 3.91 (s); 4.01 (s); 4.53 (s); 4.60 |
| | | | (s); 4.69 (s); 4.72 (s); 6.37 (m); 6.49 (m); 6.58 (m); 6.65 |
| | | | (m); 6.74 (m); 6.88 (m); 7.06-7.21 (m); 7.48 (s); 7.50 (s) |
| I-81 | | | (CDCl₃): δ = 3.05 (s); 3.93 (s); 4.04 (s); 4.60 (s); 4.65 (s); |
| | | | 4.71 (s); 4.89 (s); 6.75 (m); 6.89 (m); 7.07 (m); 7.10-7.41 |
| | | | (m); 7.49 (s); 7.50 (s) |
| I-82 | | | (CDCl₃): δ = 1.38 (bs); 1.77 (bs); 3.15 (s); 3.47 (bs); 3.68 |
| | | | (bs); 4.11 (s); 4.18 (s); 4.74 (s); 4.79 (s); 4.87 (s); 5.08 |
| | | | (s); 6.89 (m); 7.05 (m); 7.22 (m); 7.31-7.40 (m); 7.44 (m); |
| | | | 7.50 (m); 7.68 (bs) |
| I-83 | | | (CDCl₃): δ = 1.15 (t); 2.21 (s); 2.35 (s); 2.97 (s); 3.35 |
| | | | (broad); 3.93 (s); 6.58 (s); 7.15-7.35 (m). |
| I-84 | | | (CDCl₃): δ = 1.20 (t); 2.35 (s); 3.00 (s); 3.02 (s); 3.35 |
| | | | (broad); 6.22 (s); 6.80 (s); 7.15-7.35 (m); 7.40 (broad). |
| I-85 | | | (CDCl₃): δ = 1.17 (t); 2.27 (s); 2.38 (s); 2.99 (s); 3.35 |
| | | | (broad); 4.05 (s); 6.65 (d); 7.15-7.25 (m); 7.38 (broad). |
| I-86 | | | (CDCl₃): δ = 1.20 (t); 2.40 (s); 3.01 (s); 3.07 (s); 3.35 |
| | | | (broad); 6.40 (s); 6.70 (d); 7.18 (d); 7.23-7.35 (m); 7.43 (broad). |
| I-87 | | | (CDCl₃): δ = 1.23 (m); 2.38 (m); 3.02 (s); 3.35 (bs); 4.01 |
| | | | (s); 4.06 (s); 4.42 (s); 4.80 (s); 5.29 (s); 5.41 (s); 5.58 (s); |
| | | | 6.79-6.86 (m); 6.93-7.02 (m); 7.10-7.37 (m); 7.48 (bs) |
| I-88 | | | (CDCl₃): δ = 1.21 (t); 2.37 (s); 3.02 (s); 3.32 (bs); 3.51 |
| | | | (bs); 5.80 (s); 7.08 (s); 7.24-7.38 (m); 7.49 (bs); 7.56 (d) |
| I-89 | | | |
| I-90 | | | (CDCl₃): δ = 1.23 (t); 2.38 (s); 3.05 (s); 3.34 (bs); 5.73 (s); |
| | | | 6.93-7.10 (m); 7.19-7.30 (m); 7.49 (bs) |
| I-91 | | 2.79 | (CDCl₃): δ = 1.21 (m); 2.37 (s); 3.08 (bs); 3.41 (bs); 5.78 |
| | | | (s); 6.58 (m); 7.04 (m); 7.27-7.38 (m); 7.49 (bs); 7.67 (m); 7.95 (s) |
| I-92 | | 2.80 | (CDCl₃): δ = 1.21 (m); 2.38 (s); 3.05 (bs); 3.39 (bs); 5.57 |
| | | | (s); 7.03 (s); 7.08 (s); 7.21 (d); 7.46 (bs); 7.54 (d); 7.90 (s) |

HPLC-MS: column see below; Eluent: acetonitrile + 0.1% trifluoroacetic acid (TFA)/water + 0.1% TFA (gradient from 5:95 to 95:5 in 5 min at 40° C. flow of 1.8 ml/min). MS: Quadrupol Elektrospray Ionisation, 80 V (positive mode).
HPLC-1 = HPLC-column: ZOBRAX SB C18 (50 × 4.6 mm)
HPLC-2: RP-18 column (ROD 50 × 4.6 mm, Merck KgaA, Germany)

### Biological Examples for Fungicidal Activity

The fungicidal action of the compounds I was demonstrated by the following experiments:

### A. Micro Titer Tests

Active compounds have been formulated in separate DMSO stock solutions containing 10,000 ppm of the substance.

The stock solution was diluted in a microtiter plate (MTP) to indicated compound concentrations using a fungus-specific growth medium as dilution agent. Thereafter the active compound containing medium has been inoculated with an aqueous spore suspension of the respective pathogen. Inoculated MTPs were incubated at 18° C. in a vapor saturated chamber. Fungal growth was determined via absorption measurement at 405 nm on day 1 and 7 after inoculation. The measured parameters were compared to the growth of the active compound-free control variant (0 100%) and the fungus- and active compound-free blank value to determine the relative growth in % of the pathogeens in the individual active compounds.

### Use Example 1

### Fungicidal Activity Against Grey Mold Causing Fungus Botrytis cinerea

An aqueous malt-based growth medium and an aqueous spore suspension of Botrytis cinerea were used.

In this test, the samples which had been treated with 31 ppm of the active compounds I-10, I-15, I-16, I-18, I-19, I-25, I-22, I-28, I-29, I-46, I-75 and I-77, respectively, showed up to at most 10% growth of the pathogen.

### Use Example 2

### Fungicidal Activity Against Early Blight Causing Fungus Phytophthora infestans

An aqueous pea juice-based growth medium and an aqueous zoospore suspension of Phytophthora infestans were used.

In this test, the samples which had been treated with 31 ppm of the active compounds I-7, I-10, I-12, I-15, I-17, I-18, I-19, I-21, I-24, I-25, I-28, I-29, I-75, I-76, I-77, I-78, I-79 and I-80, respectively, showed up to at most 10% growth of the pathogen.

### Use Example 3

### Fungicidal Activity Against Rice Blast Causing Fungus Pyricularia oryzae

An aqueous malt-based growth medium and an aqueous spore suspension of Pyricularia oryzae were used.

In this test, the samples which had been treated with 31 ppm of the active compounds I-16, I-18, I-45, I-46, I-47, I-50, I-55, I-75 and I-77, respectively, showed up to at most 10% growth of the pathogen.

### Use Example 4

### Fungicidal Activity Against Leaf Blotch Causing Fungus Septoria tritici

An aqueous malt-based growth medium and an aqueous spore suspension of Septoria tritici were used.

In this test, the samples which had been treated with 31 ppm of the active compounds I-16, I-18, I-21, I-22, I-25, I-26, I-29, I-75 and I-77, respectively, showed up to at most 10% growth of the pathogen.

### B) Greenhouse Tests

16.8 mg of the active compounds were pre-solved in 800 μl DMSO/cyclohexanone (1:1). Thereafter 27.2 ml of an acetone/emulgator/water mixture (10% [v/v] acetone [in demineralized water], 1% [v/v] Wettol EM 31 [spreading and emulsifying agent based on ethoxylated alkylphenol], 0.5% [v/v] Silwett [spreading and emulsifying agent]) was added to the compound solution. In order to obtain the final compound concentration as indicated below this stock solution was diluted using the same acetone/emulsifier/water mixture mentioned above.

### Use Example 5

### Preventative Efficacy Against Puccinia recondita on Wheat (Wheat Brown Rust)

Wheat plants which were grown in pots have been sprayed with compounds at concentrations as indicated below and afterwards incubated at 20-25° C. and 40-80% air humidity. On the following day these wheat plants were inoculated with a spore suspension of the wheat brown rust Puccinia recondita. Plants were then incubated in a chamber for 24 h at 20-25° C. and 80-95% air humidity. During this time spores were able to germinate and germ tubes penetrated into the leaf tissue. Thereafter plants were removed from the chamber and cultivated in the glasshouse for 7 days at 20-25° C. and 40-80% air humidity. The brown rust development on wheat leafs was determined after 7 days via visual inspection and calculation of leaf surface areas (in %) showing disease symptoms.

In this test, the plants treated with 300 ppm of the compounds no. I-11, I-45, I-46, I-48, I-50, I-56 and I-60, respectively, showed an infection of less or equal to 15% whereas the untreated plants were 90% infected.

### Use Example 6

### Preventative Efficacy Against Erysiphe graminis on Wheat (Wheat Powdery Mildew)

Wheat plants which were grown in pots have been sprayed with compounds at concentrations as indicated below and afterwards incubated at 20-25° C. and 40-70% air humidity. On the following day these wheat plants were inoculated with a spore suspension of the wheat powdery mildew Erysiphe graminis. Thereafter plants were cultivated in the glasshouse for 7 days at 20-25° C. and 40-70% air humidity. The powdery mildew development on wheat leafs was determined after 7 days via visual inspection and calculation of leaf surface areas (in %) showing disease symptoms.

In this test, the plants treated with 300 ppm of the compounds no. I-38, I-39, I-44, I-45, I-46, I-47, I-50, I-60 and I-24, respectively, showed an infection of less or equal to 20% whereas the untreated plants were 90% infected.

### Use Example 7

### Preventative Efficacy Against Phytophthora infestans on Tomato (Tomato Early Blight)

Tomato plants which were grown in pots have been sprayed with compounds at concentrations as indicated below and afterwards incubated at 20-24° C. and 80-95% air humidity. On the following day these tomato plants were inoculated with a zoosporangia suspension of the tomato early blight causing oomycete Phytophthora infestans. Thereafter plants were cultivated in the glasshouse for 7 days at 20-24° C. and 80-95% air humidity. The early blight development on tomato leafs was determined after 7 days via visual inspection and calculation of leaf surface areas (in %) showing disease symptoms.

In this test, the plants treated with 600 ppm of the compounds no. I-4, I-5, I-10 and I-11, respectively, showed an infection of less or equal to 15% whereas the untreated plants were 90% infected.

### Use Example 8

### Preventative Efficacy Against Botrytis cinerea on Pepper (Grey Mold)

Pepper plants which were grown in pots have been sprayed with compounds at concentrations as indicated below and afterwards incubated at 20-25° C. and 80-95% air humidity. On the following day these pepper plants were inoculated with a spore suspension of the grey mold causing fungus Botrytis cinerea. Thereafter plants were cultivated in a dark glasshouse chamber for 5 days at 20-25° C. and 80-95% air humidity. In order to further increase the local air humidity plants were incubated in lidded boxes. The grey mold development on pepper leafs was determined after 5 days via visual inspection and calculation of leaf surface areas (in %) showing disease symptoms.

In this test, the plants treated with 600 ppm of the compounds no. I-16, I-17 and I-18, respectively, showed an infection of less or equal to 20% whereas the untreated plants were 90% infected.

### Use Example 9

### Curative Efficacy Against Phakopsora pachyrhizi on Soybeans (Soybean Rust)

Soybean plants which were grown in pots have been inoculated with a spore suspension of the soybean rust causing fungus Phakopsora pachyrhizi. Thereafter plants were cultivated in a dark glasshouse chamber for 24 h at 20-25° C. and 80-95% air humidity. During this time spores were able to germinate and germ tubes penetrated into the leaf tissue. On the following day the infected plants have been sprayed with compounds at concentrations as indicated below. Thereafter plants were cultivated in the glasshouse for 9 days at 22-27° C. and 60-80% air humidity. Rust development on soybean leafs was determined after 9 days via visual inspection and calculation of leaf surface areas (in %) showing disease symptoms.

In this test, the plants treated with 300 ppm of the compounds no. I-39, I-45, I-47, I-50, I-56, I-57 and I-24, respectively, showed an infection of less or equal to 20% whereas the untreated plants were 90% infected.

1-15. (canceled)

16. A method for combating harmful fungi, comprising:
treating the fungi or the materials, plants, the soil or seeds to be protected against fungal attack with an effective amount of at least one compound of formula I 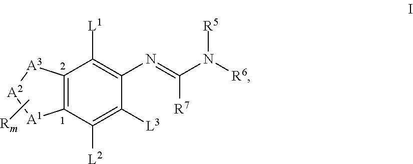
wherein:
A¹,A²,A³ independently of each other are selected from the group consisting of C, N, O and S, with the proviso that the bonds between A¹ and A², A² and A³, A¹ and carbon atom 1 of the adjacent phenyl ring, and A³ and carbon atom 2 of the adjacent phenyl ring may independently of each other be single or double bonds;
R, which may be the same or different to any other R, is hydrogen, halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
Y is a direct bond or a divalent group selected from the group consisting of —O—, —S(═O)ₚ—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y₁)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —(Y¹)ₖ—S(═O)ₚ—, —S(═O)ₚ—(Y¹)ₖ—, —O—Y²—, —N(R^{d})—Y²—, —O—Y²—(Y¹)ₖ—, —O—Y²—NR^{d}—NR^{d}—, —O—Y²—N(R^{d})—, —O—Y²—N(R^{d})—Y²—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—S(═O)ₚ—(Y¹)ₖ—, —(Y¹)ₖ—Y⁴—(Y¹)ₖ—O—, —(Y¹)ₖ—Y⁴—(Y¹)ₖ—S(═O)ₚ—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
p is 0, 1 or 2,
k is an integer between 1 and 8,
Y¹ is —C(R^{e})₂—,
Y² is —C(═S)— or —C(═O)—,
Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁴ is —O—N═C(R^{e})—, where the bond depicted on the right side of the divalent group Y⁴ is attached to the group being depicted on the right side of Y⁴ in the definition of Y, or, if no such group is depicted on the right side of Y⁴, the bond depicted on the right side of Y⁴ is attached to R¹;
Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group consisting of N, O and S;
R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
R^{e} is hydrogen, CN, NH₂, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S, and wherein the aforementioned cyclic groups R^{e} may be attached directly or via an oxygen or sulfur atom,
or
—[O—(ZR^{f}₂)ₙ]ₒ—R^{f}, wherein
Z are independently of each other C or Si,
R^{f} is hydrogen, C₁-C₈-alkyl, phenyl or benzyl,
n is an integer between 1 and 5,
o is an integer between 3 and 10;
R⁵,R⁶ independently of one another are C₁-C₈-alkyl, C₁-C₈-haloalkyl, C₁-C₄-alkoxy or C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S; and where R⁵ and R⁶ in each case together with the nitrogen atom linking them may form a five- to ten-membered saturated or partially unsaturated heterocyclyl which, in addition to the carbon atoms, may contain 1 to 3 heteroatoms selected from the group consisting of N, O and S;
R⁷ is hydrogen, C₁-C₈-alkyl, C₁-C₈-haloalkyl, C₁-C₈-alkoxy or C₁-C₈-haloalkoxy;
L¹,L²,L³ independently of one another are hydrogen, halogen, OH, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
and
aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from the group consisting of:
R^{a} is amino, halogen, hydroxyl, oxo, nitro, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, carbonyloxy, NR^{A}R^{B}, C₂-C₈-alkylene, C₂-C₈-oxyalkylene, C₁-C₈-oxyalkyleneoxy, phenyl, naphthyl or a three- to ten-membered saturated, partially unsaturated or aromatic mono- or bicyclic heterocyclyl or which, in addition to carbon atoms, contains one to four heteroatoms selected from the group consisting of O, N and S as ring members; and wherein the aforementioned phenyl and heterocyclyl groups R^{a} are attached via a direct bond, an oxygen or sulfur atom;
and
two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group consisting of N, O and S, and
where the aliphatic or cyclic groups R^{a} for their part may carry 1, 2, 3 or up to the maximum possible number of identical or different groups R^{b}:
R^{b} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
where the cyclic groups R^{b} for their part may carry 1, 2, 3 or up to the maximum possible number of identical or different groups R^{c}:
R^{c} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
R^{A},R^{B} independently of one another are CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, NR^{C}R^{D}, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{A} and/or R^{B} may for their part be attached directly or via a nitrogen or oxygen atom;
where the aliphatic or cyclic groups R^{A} and/or R^{B} for their part may carry 1, 2, 3 or up to the maximum possible number of identical or different groups R_{b},
R^{B} may additionally be hydrogen;
R^{C},R^{D} independently of one another are hydrogen, CN, carboxyl, C₁-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{C} and/or R^{D} may for their part be attached directly or via a nitrogen or oxygen atom;
or the N-oxide or agriculturally acceptable salt thereof.

17. The method of claim 16, wherein plant propagation material, seed, the seedlings' roots or shoots are protected from infestation by harmful fungi.

18. Seed coated with at least one compound of formula I, 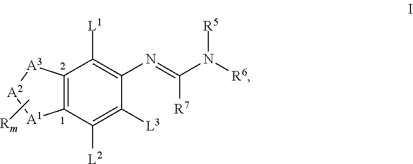
wherein:
A¹,A²,A³ independently of each other are selected from the group consisting of C, N, O and S, with the proviso that the bonds between A¹ and A², A² and A³, A¹ and carbon atom 1 of the adjacent phenyl ring, and A³ and carbon atom 2 of the adjacent phenyl ring may independently of each other be single or double bonds;
R, which may be the same or different to any other R, is hydrogen, halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
Y is a direct bond or a divalent group selected from the group consisting of —O—, —S(═O)ₚ—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —(Y¹)ₖ—S(═O)ₚ—, —S(═O)ₚ—(Y¹)ₖ—, —O—Y²—, —N(R^{d})—Y²—, —O—Y²—(Y¹)ₖ—, —O—Y²—NR^{d}—NR^{d}—, —O—Y²—N(R^{d})—, —O—Y²—N(R^{d})—Y²—, —(Y¹)ₖ—Y⁴—, —(Y¹)ₖ—Y⁴—Y³—, —(Y¹)ₖ—Y⁴—N(R^{d})—, (Y¹)ₖ—Y⁴—O—(Y¹)ₖ—, —(Y¹)ₖ—Y⁴—S(═O)ₚ—(Y¹)ₖ—, (Y¹)ₖ—Y⁴—(Y¹)ₖ—O—, (Y¹)ₖ—Y⁴—(Y¹)ₖ—S(═O)ₚ—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
p is 0, 1 or 2,
k is an integer between 1 and 8,
Y¹ is —C(R^{e})₂—,
Y² is —C(═S)— or C(═O)—,
Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁴ is —O—N═C(R^{e})—, where the bond depicted on the right side of the divalent group Y⁴ is attached to the group being depicted on the right side of Y⁴ in the definition of Y, or, if no such group is depicted on the right side of Y⁴, the bond depicted on the right side of Y⁴ is attached to R¹;
Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group consisting of N, O and S;
R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
R^{e} is hydrogen, CN, NH₂, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S, and wherein the aforementioned cyclic groups R^{e} may be attached directly or via an oxygen or sulfur atom,
or
—[O—(ZR^{f}₂)ₙ]ₒ—R^{f}, wherein
Z are independently of each other C or Si,
R^{f} is hydrogen, C₁-C₈-alkyl, phenyl or benzyl,
n is an integer between 1 and 5,
o is an integer between 3 and 10;
R⁵,R⁶ independently of one another are C₁-C₈-alkyl, C₁-C₈-haloalkyl, C₁-C₄-alkoxy or C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S; and where R⁵ and R⁶ in each case together with the nitrogen atom linking them may form a five- to ten-membered saturated or partially unsaturated heterocyclyl which, in addition to the carbon atoms, may contain 1 to 3 heteroatoms selected from the group consisting of N, O and S;
R⁷ is hydrogen, C₁-C₈-alkyl, C₁-C₈-haloalkyl, C₁-C₈-alkoxy or C₁-C₈-haloalkoxy;
L¹,L²,L³ independently of one another are hydrogen, halogen, OH, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
and
aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from the group consisting of:
R^{a} is amino, halogen, hydroxyl, oxo, nitro, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, NR^{A}R^{B}, C₂-C₈-alkylene, C₂-C₈-oxyalkylene, C₁-C₈-oxyalkyleneoxy, phenyl, naphthyl or a three- to ten-membered saturated, partially unsaturated or aromatic mono- or bicyclic heterocyclyl or which, in addition to carbon atoms, contains one to four heteroatoms selected from the group consisting of O, N and S as ring members; and wherein the aforementioned phenyl and heterocyclyl groups R^{a} are attached via a direct bond, an oxygen or sulfur atom;
and
two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group consisting of N, O and S, and
where the aliphatic or cyclic groups R^{a} for their part may carry 1, 2, 3 or up to the maximum possible number of identical or different groups R^{b}:
R^{b} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
where the cyclic groups R^{b} for their part may carry 1, 2, 3 or up to the maximum possible number of identical or different groups R^{c}:
R^{c} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
R^{A},R^{B} independently of one another are CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, NR^{C}R^{D}, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{A} and/or R^{B} may for their part be attached directly or via a nitrogen or oxygen atom;
where the aliphatic or cyclic groups R^{A} and/or R^{B} for their part may carry 1, 2, 3 or up to the maximum possible number of identical or different groups R^{b},
R^{B} may additionally be hydrogen;
R^{C},R^{D} independently of one another are hydrogen, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{C} and/or R^{D} may for their part be attached directly or via a nitrogen or oxygen atom;
or the N-oxide or agriculturally acceptable salt thereof
or a composition comprising it thereof, in an amount of from 0.1 g to 10 kg per 100 kg of seed.

19. A compound of formula I 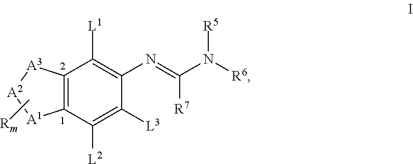
wherein:
A¹,A²,A³ independently of each other are selected from the group consisting of C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is hydrogen, halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
Y is a direct bond or a divalent group selected from —O—, —S(═O)ₚ—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—S(═)ₚ—, —S(═O)ₚ—(Y¹)ₖ—, —O—Y²—, —N(R^{d})—Y²—, —O—Y²—(Y¹)ₖ—, —S—(Y¹)ₖ—Y³—, —(Y¹)ₖ—S(═O)ₚ—, —S(═O)ₚ—(Y¹)ₖ—, —O—Y²—, —N(R^{d})—Y²—, —O—Y²—(Y¹)ₖ—, —O—Y²—NR^{d}—NR^{d}—, —O—Y²—N(R^{d})—, —O—Y²—N(R^{d})—Y²—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—S(═O)ₚ—(Y¹)ₖ—, —(Y¹)ₖ—Y⁴—(Y¹)ₖ—O—, —(Y¹)ₖ—Y⁴—(Y¹)ₖ—S(═O)ₚ—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
p is 0, 1 or 2,
k is an integer between 1 and 8,
Y¹ is —C(R^{e})₂—,
Y² is —C(═S)— or —C(═O)—,
Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁴ is —O—N═C(R^{e})—, where the bond depicted on the right side of the divalent group Y⁴ is attached to the group being depicted on the right side of Y⁴ in the definition of Y, or, if no such group is depicted on the right side of Y⁴, the bond depicted on the right side of Y⁴ is attached to R¹;
Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group consisting of N, O and S;
R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
R^{e} is hydrogen, CN, NH₂, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S, and wherein the aforementioned cyclic groups R^{e} may be attached directly or via an oxygen or sulfur atom,
or
—[O—(ZR^{f}₂)ₙ]ₒ—R^{f}, wherein
Z are independently of each other C or Si,
R^{f} is hydrogen, C₁-C₈-alkyl, phenyl or benzyl,
n is an integer between 1 and 5,
o is an integer between 3 and 10;
R⁵,R⁶ independently of one another are C₁-C₈-alkyl, C₁-C₈-haloalkyl, C₁-C₄-alkoxy or C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S; and
where R⁵ and R⁶ in each case together with the nitrogen atom linking them may form a five- to ten-membered saturated or partially unsaturated heterocyclyl which, in addition to the carbon atoms, may contain 1 to 3 heteroatoms selected from the group consisting of N, O and S;
R⁷ is hydrogen, C₁-C₈-alkyl, C₁-C₈-haloalkyl, C₁-C₈-alkoxy or C₁-C₈-haloalkoxy;
L¹,L² independently of one another are hydrogen, halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
L³ is halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
with the proviso that L¹ and L³ may not be both CN;
and
with the proviso that in case A³ is N or C, one of A¹ and A³ is C, the other one of both being N, one of the bonds between A¹ and A² and between A² and A³ is a double bond and the aforementioned atoms form together with the phenyl ring a 1H-indazole or 1H-indole: L³ may in addition be hydrogen;
aliphatic and cyclic groups R⁴ to R⁷, Y⁵, R^{d}, R^{e} and L¹ to L³ may carry 1, 2, 3, 4 or up to the maximum possible number of identical or different groups R^{a}:
R^{a} is independently of one another amino, halogen, hydroxyl, oxo, nitro, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, NR^{A}R^{B}, C₂-C₈-alkylene, C₂-C₈-oxyalkylene, C₁-C₈-oxyalkyleneoxy, phenyl, naphthyl or a three- to ten-membered saturated, partially unsaturated or aromatic mono- or bicyclic heterocyclyl or which, in addition to carbon atoms, contains one to four heteroatoms selected from the group consisting of O, N and S as ring members; and wherein the aforementioned phenyl and heterocyclyl groups R^{a} are attached via a direct bond, an oxygen or sulfur atom;
and
two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group consisting of N, O and S, and
where the aliphatic or cyclic groups R^{a} for their part may carry 1, 2, 3 or up to the maximum possible number of identical or different groups R^{b}:
R^{b} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
where the cyclic groups R^{b} for their part may carry 1, 2, 3 or up to the maximum possible number of identical or different groups R^{c}:
R^{c} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
R^{A},R^{B} independently of one another are CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, NR^{C}R^{D}, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{A} and/or R^{B} may for their part be attached directly or via a nitrogen or oxygen atom;
where the aliphatic or cyclic groups R^{A} and/or R^{B} for their part may carry 1, 2, 3 or up to the maximum possible number of identical or different groups R^{b},
R^{B} may additionally be hydrogen;
R^{C},R^{D} independently of one another are hydrogen, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{C} and/or R^{D} may for their part be attached directly or via a nitrogen or oxygen atom;
except for N-(1-diethoxymethyl-1H-indazol-6-yl)-N′-methyl-formamidine (CAS registry no. 65258-27-7), N-(1-diethoxymethyl-1H-indazol-5-yl)N′-methyl-formamidine (65258-39-1), N′-(1-diethoxymethyl-1H-indazol-6-yl)-N—[N-(2,4-dimethylphenyl)formidoyl]-N-methyl-formamidine (65259-25-8), (N′-(1-diethoxymethyl-1H-indazol-5-yl)-N—[N-(2,4-dimethyl-phenyl)formidoyl]-N-methyl-formamidine (65258-91-5), N′-(1-diethoxymethyl-1H-indazol-6-yl)-N—[N-(4-chloro-2-methylphenyl)formidoyl]-N-methyl-formamidine (65531-27-3), N′-(1-diethoxymethyl-1H-indazol-5-yl)-N—[N-(4-chloro-2-methyl-phenyl)formidoyl]-N-methyl-formamidine (65258-92-6), N′-(1H-indazol-6-yl)-N,N-diethyl-formamidine (67236-60-6), N′-(1,2-dimethyl-3-ethoxycarbonyl-6-hydroxy-1H-indol-5-yl)-N,N-dimethylformamidine (136265-46-8) and N′-(2-[[4-[(1-methylethyl)amino]-pyrdin-2-yl]-1-piperazinyl]carbonyl)-1H-indol-5-yl)-N,N-dimethyl-formamidine (144674-89-5);
or an N-oxide or an agriculturally acceptable salt thereof.

20. The compound according to claim 19, wherein:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is hydrogen, halogen, CN, oxo, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl or Y—R⁴;
m indicates the number of the substituents R and m is 0, 1, 2, 3 or 4;
R⁴ is C₁-C₈-alkyl, C₂-C₈-alkenyl, C₂-C₈-alkynyl, C₃-C₈-cycloalkyl, C₃-C₈-cycloalkenyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
Y is a direct bond or a divalent group selected from —O—, —S(═O)ₚ—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —(Y¹)ₖ—S(═O)ₚ—, —S(═O)ₚ—(Y¹)ₖ—, —O—Y²—, —N(R^{d})—Y²—, —O—Y²—(Y¹)ₖ—, —O—Y²—NR^{d}—NR^{d}—, —O—Y²—N(R^{d})—, —O—Y²—N(R^{d})—Y²—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—S(═O)ₚ—(Y¹)ₖ—, —(Y¹)ₖ—Y⁴—(Y¹)ₖ—O—, —(Y¹)ₖ—Y⁴—(Y¹)ₖ—S(═O)ₚ—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
p is 0, 1 or 2,
k is an integer between 1 and 8,
Y¹ is —C(R^{e})₂—,
Y² is —C(═S)— or C(═O)—,
Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁴ is —O—N═C(R^{e})—, where the bond depicted on the right side of the divalent group Y⁴ is attached to the group being depicted on the right side of Y⁴ in the definition of Y, or, if no such group is depicted on the right side of Y⁴, the bond depicted on the right side of Y⁴ is attached to R¹;
Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group consisting of N, O and S;
R^{d} is hydrogen, C₁-C₈-alkyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
R^{e} is hydrogen, CN, NH₂, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S, and wherein the aforementioned cyclic groups R^{e} may be attached directly or via an oxygen or sulfur atom,
or
—[O—(ZR^{f}₂)ₙ]ₒ—R^{f}, wherein
Z are independently of each other C or Si,
R^{f} is hydrogen, C₁-C₈-alkyl, phenyl or benzyl,
n is an integer between 1 and 5,
o is an integer between 3 and 10;
R⁵,R⁶ independently of one another are C₁-C₈-alkyl, C₁-C₈-haloalkyl, C₁-C₄-alkoxy or C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S; and
where R⁵ and R⁶ in each case together with the nitrogen atom linking them may form a five- to ten-membered saturated or partially unsaturated heterocyclyl which, in addition to the carbon atoms, may contain 1 to 3 heteroatoms selected from the group consisting of N, O and S;
R⁷ is hydrogen, C₁-C₈-alkyl, C₁-C₈-haloalkyl, C₁-C₈-alkoxy or C₁-C₈-haloalkoxy;
L¹,L² independently of one another are hydrogen, halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S;
L³ is halogen, CN, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C₂-C₄-haloalkynyl, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group of consisting N, O and S;
with the proviso that L¹ and L³ may not be both CN;
aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from:
R^{a} is amino, halogen, hydroxyl, oxo, nitro, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₈-alkylcarbonyloxy, NR^{A}R^{B}, C₂-C₈-alkylene, C₂-C₈-oxyalkylene, C₁-C₈-oxyalkyleneoxy, phenyl, naphthyl or a three- to ten-membered saturated, partially unsaturated or aromatic mono- or bicyclic heterocyclyl or which, in addition to carbon atoms, contains one to four heteroatoms selected from the group consisting of O, N and S as ring members; and wherein the aforementioned phenyl and heterocyclyl groups R^{a} are attached via a direct bond, an oxygen or sulfur atom;
and
two radicals R^{a} that are bound to adjacent ring member atoms of the cyclic group Y⁵ may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group consisting of N, O and S, and
where the aliphatic or cyclic groups R^{a} for their part may carry 1, 2, 3 or up to the maximum possible number of identical or different groups R^{b}:
R^{b} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy, C₁-C₄-haloalkoxy, C₁-C₈-alkylcarbonylamino, phenyl, phenoxy, pyridyl, pyridyloxy or C₃-C₈-cycloalkylcarbonylamino;
where the cyclic groups R^{b} for their part may carry 1, 2, 3 or up to the maximum possible number of identical or different groups R^{c}:
R^{c} is halogen, hydroxyl, nitro, CN, carboxyl, C₁-C₄-alkyl, C₂-C₄-alkenyl, C₂-C₈-alkynyl, C₁-C₄-haloalkyl, C₁-C₄-alkoxy or C₁-C₄-haloalkoxy;
R^{A},R^{B} independently of one another are CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, NR^{C}R^{D}, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{A} and/or R^{B} may for their part be attached directly or via a nitrogen or oxygen atom;
where the aliphatic or cyclic groups R^{A} and/or R^{B} for their part may carry 1, 2, 3 or up to the maximum possible number of identical or different groups R^{b},
R^{B} may additionally be hydrogen;
R^{C},R^{D} independently of one another are hydrogen, CN, carboxyl, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₃-C₈-cycloalkenyl, C₂-C₆-alkenyloxy, C₃-C₆-alkynyloxy, C₁-C₄-alkoxyimino, C₂-C₈-alkylidene, C₃-C₈-cycloalkylidene, C₁-C₁₂-alkylamino, C₁-C₄-alkoxycarbonyl, C₁-C₄-alkylcarbonyloxy, C₂-C₈-alkylene, C₂-C₈-oxyalkylene, C₁-C₈-oxyalkyleneoxy, phenyl, naphthyl or a 3- to 10-membered saturated, partially unsaturated or aromatic mono- or bicyclic heterocyclyl which, in addition to carbon atoms, contains 1 to 4 heteroatoms selected from the group consisting of O, N and S as ring members;
where the cyclic groups R^{C} and/or R^{D} may for their part be attached directly or via a nitrogen or oxygen atom.

21. The compound according to claim 20, wherein:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds;
R, which may be the same or different to any other R, is halogen, CN, oxo, C₁-C₆-alkyl, C₁-C₆-haloalkyl, C₁-C₆-alkoxy, C₁-C₆-haloalkoxy, di(C₁-C₆-alkyl)amino, C₁-C₆-alkylthio, C₁-C₆-haloalkylthio, C₁-C₆-alkoxy-C₁-C₄-alkyl, C₁-C₆-haloalkoxy-C₁-C₄-alkyl, or Y—R⁴;
m indicates the number of the substituents R and m is 1 or 2;
R⁴ is C₁-C₈-alkyl, C₂-C₈-alkenyl, C₂-C₈-alkynyl, C₃-C₈-cycloalkyl, C₃-C₈-cycloalkyl, C₃-C₈-cycloalkenyl, phenyl or a 3- to 10-membered saturated, partially unsaturated or aromatic monocyclic heterocyclyl wherein the ring member atoms of the heterocyclyl include besides carbon atoms 1, 2, 3 or 4 heteroatoms selected from the group consisting of N, O and S;
Y is a direct bond or a divalent group selected from —O—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴, —(Y¹)ₖ—Y³—, —O—(Y¹)ₖ—Y³—, —S—(Y¹)ₖ—Y³—, —O—Y²—, —N(R^{d})—Y²—, —O—Y²—(Y¹)ₖ—, —O—Y²—N(R^{d})—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—(Y¹)ₖ—O—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
k is an integer between 1 and 3,
Y¹ is —C(R^{e})₂—,
Y² is —C(═O)—,
Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group consisting of N, O and S;
R^{d} is hydrogen, C₁-C₈-alkyl or phenyl;
R^{e} is hydrogen, CN, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S, and wherein the aforementioned cyclic groups R^{e} may be attached directly or via an oxygen or sulfur atom,
R⁵,R⁶ independently of one another are C₁-C₈-alkyl, C₁-C₈-haloalkyl, C₁-C₄-alkoxy or C₃-C₈-cycloalkyl, and where R⁵ and R⁶ in each case together with the nitrogen atom linking them may form a five- to ten-membered saturated or partially unsaturated ring;
R⁷ is hydrogen, C₁-C₈-alkyl, C₁-C₈-haloalkyl, C₁-C₈-alkoxy or C₁-C₈-haloalkoxy;
L¹,L² independently of one another are hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-haloalkoxy-C₁-C₄-alkyl, C₃-C₈-cycloalkyl;
L³ is halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkoxy-C₁-C₄-alkyl, C₁-C₆-haloalkoxy-C₁-C₄-alkyl, C₃-C₈-cycloalkyl;
and
aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from:
R^{a} is halogen, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₂-C₆-alkenyloxy, C₃-C₆-alkynyloxy, phenyl; and wherein the aforementioned phenyl groups R^{a} are attached via a direct bond, an oxygen or sulfur atom;
and
where the aliphatic or cyclic groups R^{a} for their part may carry 1, 2, 3 or up to the maximum possible number of identical or different groups R^{b}:
R^{b} is halogen, CN, C₁-C₄-haloalkyl, C₁-C₄-alkoxy, C₁-C₄-haloalkoxy, phenyl, phenoxy, pyridyl or pyridyloxy;
where the cyclic groups R^{b} for their part may carry 1, 2, 3 or up to the maximum possible number of identical or different groups R^{c}:
R^{c} is halogen, CN, C₁-C₄-haloalkyl, C₁-C₄-alkoxy or C₁-C₄-haloalkoxy;
R^{A},R^{B} independently of one another are C C₁-C₄-haloalkyl, C₃-C₆-cycloalkyl, phenyl;
where the cyclic groups R^{A} and/or R^{B} may for their part be attached directly or via a nitrogen or oxygen atom;
where the aliphatic or cyclic groups R^{A} and/or R^{B} for their part may carry 1, 2, 3 or up to the maximum possible number of identical or different groups R^{b},
R^{B} may additionally be hydrogen;
R^{C},R^{D} independently of one another are hydrogen, C₁-C₄-alkyl, C₁-C₄-haloalkyl, C₁-C₄-alkoxy, C₁-C₄-haloalkoxy, C₃-C₆-cycloalkyl, phenyl,
where the cyclic groups R^{C} and/or R^{D} may for their part be attached directly or via a oxygen atom.

22. The compound according to claim 19 wherein R⁷ is hydrogen.

23. The compound according to claim 19 wherein Y is a direct bond or a divalent group selected from —O—, —(Y¹)ₖ—, —O—(Y¹)ₖ—, and —O—(Y¹)ₖ—O—.

24. The compound according to claim 19, wherein m is 1, 2, 3 or 4 and at least one substituent R is defined as 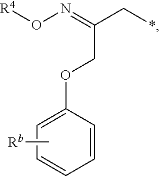
wherein * indicates the bond to any of the atoms A¹, A² or A³.

25. The compound according to claim 19, which is selected from the group consisting of: 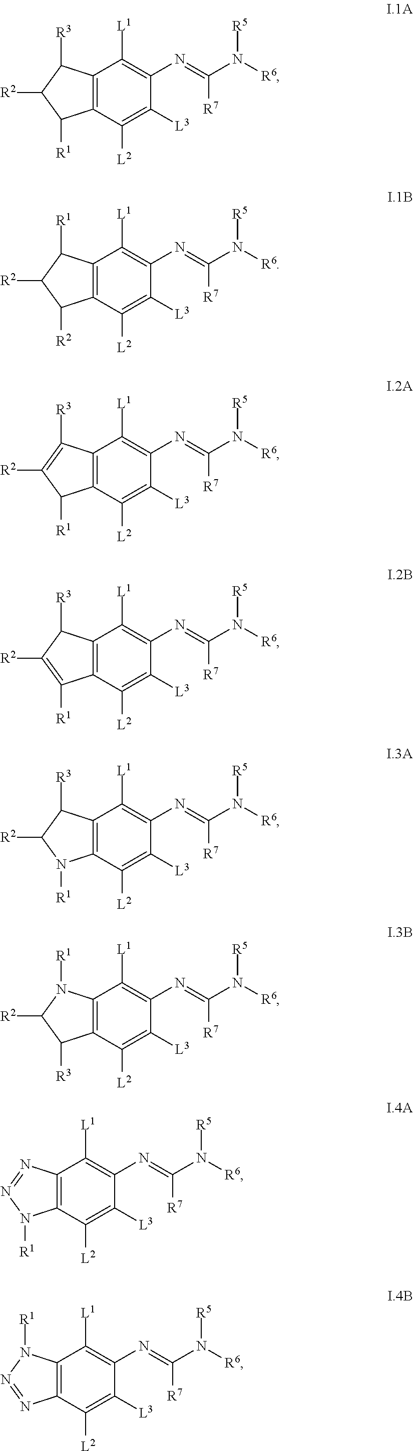 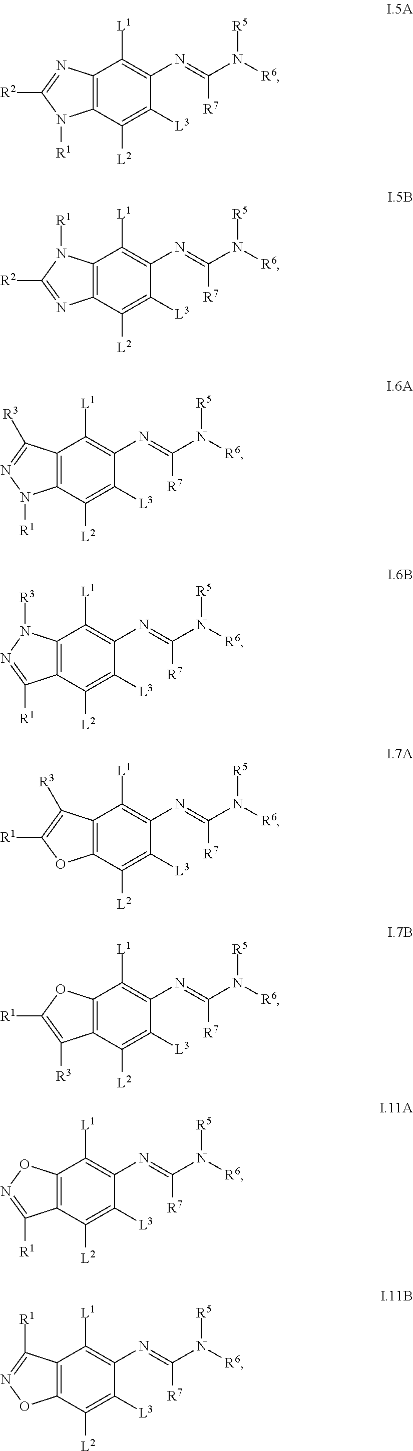 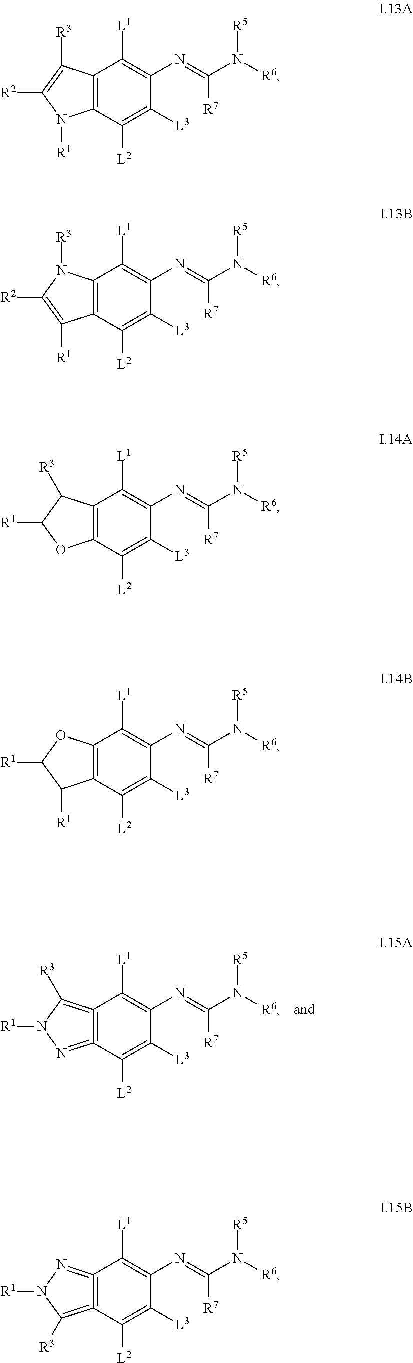
wherein A¹, A² and A³ are as defined in the sub-formulae and R¹, R² and R³ are independently hydrogen or R.

26. A process for preparing the compound of claim 19, which comprises reacting a compound of formula II 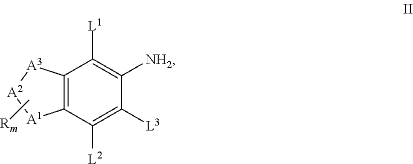
in presence of a catalyst with a compound of formula IIIc

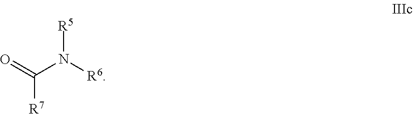

27. A process for preparing in the compound of claim 19, which comprises reacting a compound of formula II 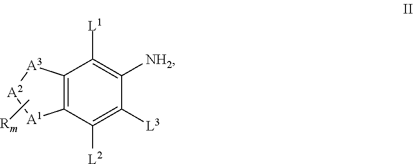
with a compound of formula IIId

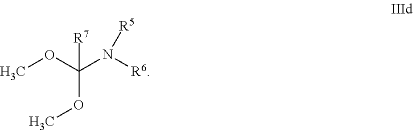

28. An agrochemical composition comprising a solvent or solid carrier and at least one compound of claim 19.

29. Composition according to claim 28, comprising a further active compound.

30. The method of claim 16, wherein, in the compound of formula I:
A¹,A²,A³ independently of each other are selected from C, N, O and S, with the proviso that the bonds between A¹ and A², A² and A³, A¹ and carbon atom 1 of the adjacent phenyl ring, and A³ and carbon atom 2 of the adjacent phenyl ring may independently of each other single or double bonds; R, which may be the same or different to any other R, is halogen, CN, oxo, C₁-C₆-alkyl, C₁-C₆-haloalkyl, C₁-C₆-alkoxy, C₁-C₆-haloalkoxy, di(C₁-C₆-alkyl)amino, C₁-C₆-alkylthio, C₁-C₆-haloalkylthio, C₁-C₆-alkoxy-C₁-C₄-alkyl, C₁-C₆-haloalkoxy-C₁-C₄-alkyl, or Y—R⁴;
m indicates the number of the substituents R and m is 1 or 2;
R⁴ is C₁-C₈-alkyl, C₂-C₈-alkenyl, C₂-C₈-alkynyl, C₃-C₈-cycloalkyl, C₃-C₈-cycloalkyl, C₃-C₈-cycloalkenyl, phenyl or a 3- to 10-membered saturated, partially unsaturated or aromatic monocyclic heterocyclyl wherein the ring member atoms of the heterocyclyl include besides carbon atoms 1, 2, 3 or 4 heteroatoms selected from the group consisting of N, O and S;
Y is a direct bond or a divalent group selected from —O—, —N(R^{d})—, —CR^{e}═CR^{e}—, —C≡C—, —(Y¹)ₖ—, —O—(Y¹)ₖ—, —O—(Y¹)ₖ—O—, —Y²—, —(Y¹)ₖ—Y²—, —(Y¹)ₖ—Y²—(Y¹)ₖ—, —(Y¹)ₖ—Y²—(Y¹)ₖ—O—, —Y³—, —Y⁴—, —(Y¹)ₖ—Y³—, —O—(Y¹)ₖ—Y³—, —S—(Y¹)ₖ—Y³—, —O—Y²—, —N(R^{d})—Y²—, —O—Y²—(Y¹)ₖ—, —O—Y²—N(R^{d})—, —(Y¹)ₖ—Y⁴—, —(Y¹)ₖ—Y⁴—Y³—, —(Y¹)ₖ—Y⁴—N(R^{d})—, —(Y¹)ₖ—Y⁴—O—(Y¹)ₖ—, —(Y¹)ₖ—Y⁴—(Y¹)ₖ—O—, —Y⁵—, —O—Y⁵—, —Y⁵—O—, —(Y¹)ₖ—Y⁵—, —(Y¹)ₖ—Y⁵—(Y¹)ₖ—, —(Y¹)ₖ—Y⁵—O—, —O—Y⁵—(Y¹)ₖ—, —Y⁵—(Y¹)ₖ—Y³—, —(Y¹)ₖ—Y³—Y⁵—, —(Y¹)ₖ—Y³—Y⁵—Y³— and —(Y¹)ₖ—Y³—Y⁵—(Y¹)ₖ—Y³—; where the bond depicted on the right side of the divalent group Y is attached to R⁴, and where:
k is an integer between 1 and 3,
Y¹ is —C(R^{e})₂—,
Y² is —C(═O)—,
Y³ is —C(R^{e})═N—O—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁴ is —O—N═C(R^{e})—, where the bond depicted on the right side of the divalent group Y³ is attached to the group being depicted on the right side of Y³ in the definition of Y, or, if no such group is depicted on the right side of Y³, the bond depicted on the right side of Y³ is attached to R¹;
Y⁵ is C₃-C₈-cycloalkylene, C₃-C₈-cycloalkenylene, phenylene, a 5-, 6-, or 7-membered saturated or partially unsaturated heterocyclylene or a 5- or 6-membered heteroarenediyl, wherein the ring member atoms of the heterocyclylene or heteroarenediyl include besides carbon atoms 1, 2, 3 or 4 heteroatoms selected from the group consisting of N, O and S;
R^{d} is hydrogen, C₁-C₈-alkyl or phenyl;
R^{e} is hydrogen, CN, C₁-C₈-alkyl, C₂-C₈-alkenyl, C₂-C₈-alkynyl, C₁-C₈-alkoxy, C₃-C₈-cycloalkyl, C₃-C₈-cycloalkenyl, phenyl or a 3- to 10-membered saturated, partially unsaturated or aromatic mono- or bicyclic heterocyclyl wherein the ring member atoms of the heterocyclyl include besides carbon atoms 1, 2, 3 or 4 heteroatoms selected from the group consisting of N, O and S, and wherein the aforementioned cyclic groups R^{e} may be attached directly or via an oxygen or sulfur atom,
R⁵,R⁶ independently of one another are C₁-C₈-alkyl, C₁-C₈-haloalkyl, C₁-C₄-alkoxy or C₃-C₈-cycloalkyl, and where R⁵ and R⁶ in each case together with the nitrogen atom linking them may form a five- to ten-membered saturated or partially unsaturated ring;
R⁷ is hydrogen, C₁-C₈-alkyl, C₁-C₈-haloalkyl, C₁-C₈-alkoxy or C₁-C₈-haloalkoxy;
L¹,L² independently of one another are hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-haloalkoxy-C₁-C₄-alkyl, C₃-C₈-cycloalkyl;
L³ is halogen, CN, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkoxy-C₁-C₄-alkyl, C₁-C₆-haloalkoxy-C₁-C₄-alkyl, C₃-C₈-cycloalkyl;
and
aliphatic and cyclic groups R⁴ to R⁷, Y⁵, R^{d}, R^{e} and L¹ to L³ may carry 1, 2, 3, 4 or up to the maximum possible number of identical or different groups R^{a} which independently of one another are selected from:
R^{a} is halogen, C₁-C₄-alkyl, C₂-C₄-alkenyl, C₂-C₄-alkynyl, C₁-C₄-haloalkyl, C₁-C₄-alkoxy, C₁-C₄-haloalkoxy, C₃-C₆-cycloalkyl, C₂-C₆-alkenyloxy, C₃-C₆-alkynyloxy, phenyl; and wherein the aforementioned phenyl groups R^{a} are attached via a direct bond, an oxygen or sulfur atom;
and
where the aliphatic or cyclic groups R^{a} for their part may carry 1, 2, 3 or up to the maximum possible number of identical or different groups R^{b}:
R^{b} is halogen, CN, C₁-C₄-haloalkyl, C₁-C₄-alkoxy, C₁-C₄-haloalkoxy, phenyl, phenoxy, pyridyl or pyridyloxy;
where the cyclic groups R^{b} for their part may carry 1, 2, 3 or up to the maximum possible number of identical or different groups R^{c}:
R^{c} is halogen, CN, C₁-C₄-alkyl, C₁-C₄-haloalkyl, C₁-C₄-alkoxy or C₁-C₄-haloalkoxy;
R^{A},R^{B} independently of one another are C₁-C₄-alkyl, C₁-C₄-haloalkyl, C₃-C₆-cycloalkyl, phenyl;
where the cyclic groups R^{A} and/or R^{B} may for their part be attached directly or via a nitrogen or oxygen atom;
where the aliphatic or cyclic groups R^{A} and/or R^{B} for their part may carry 1, 2, 3 or up to the maximum possible number of identical or different groups R^{b},
R^{B} may additionally be hydrogen;
R^{C},R^{D} independently of one another are hydrogen, C₁-C₄-alkyl, C₁-C₄-haloalkyl, C₁-C₄-alkoxy, C₁-C₄-haloalkoxy, C₃-C₆-cycloalkyl, phenyl,
where the cyclic groups R^{C} and/or R^{D} may for their part be attached directly or via a oxygen atom.

31. The method of claim 16, wherein, in the compound of formula I, R⁷ is hydrogen.

32. The method of claim 16, wherein, in the compound of formula I, Y is a direct bond or a divalent group selected from —O—, —(Y¹)ₖ—, —O—(Y¹)ₖ—, and —O—(Y¹)ₖ—O—.

33. The compound according to claim 19, wherein m is 1, 2, 3 or 4 and at least one substituent R is defined as 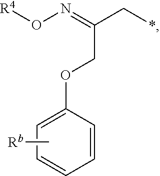
wherein * indicates the bond to any of the atoms A¹, A² or A³.

34. The method of claim 16, wherein the compound of formula I is selected from the group consisting of: 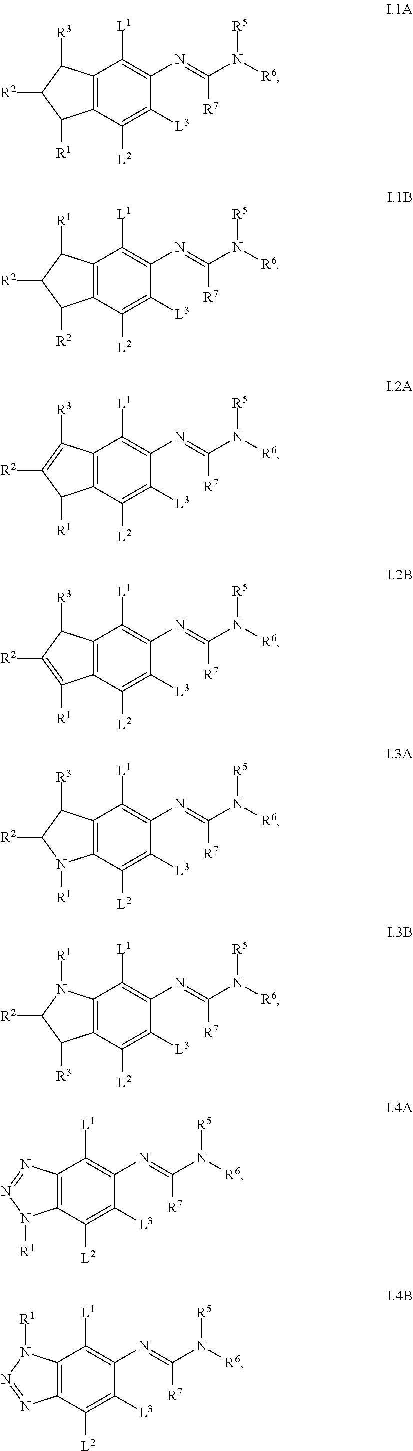 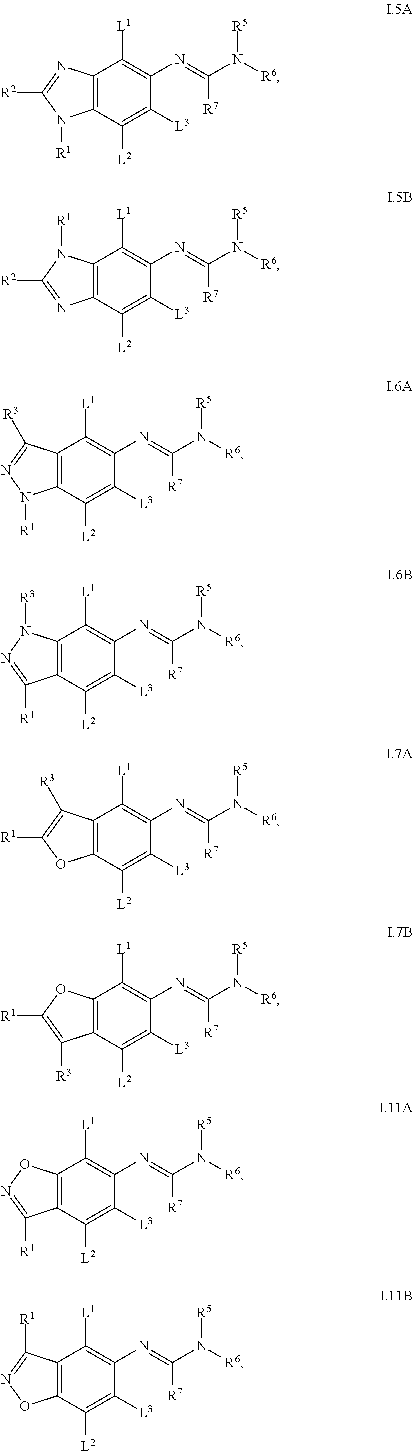 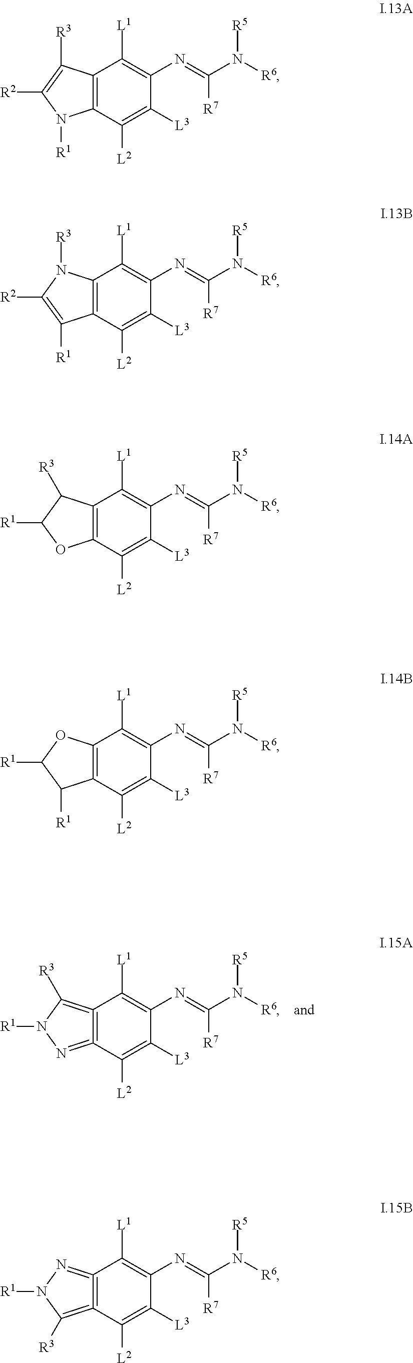
wherein A¹, A² and A³ are as defined in the sub-formulae and R¹, R² and R³ are independently hydrogen or R.
